(19) United States
(12) Patent Application Publication
(10) Pub. No.: US 2013/0303575 A1
(43) Pub. Date: Nov. 14, 2013

(54) ADMINISTRATION OF AN INHIBITOR OF HDAC

(71) Applicants: Konrad BURKE, Madison, CT (US); Peter ORDENTLICH, San Diego, CA (US); Robert S. Goodenow, San Clemente, CA (US)

(72) Inventors: Konrad BURKE, Madison, CT (US); Peter ORDENTLICH, San Diego, CA (US); Robert S. Goodenow, San Clemente, CA (US)

(73) Assignee: SYNDAX PHARMACEUTICALS, INC., Waltham, MA (US)

(21) Appl. No.: 13/940,137

(22) Filed: Jul. 11, 2013

Related U.S. Application Data
(63) Continuation of application No. 12/743,557, filed on Aug. 9, 2010, now abandoned.
(60) Provisional application No. 60/989,082, filed on Nov. 19, 2007.

(51) Int. Cl. A61K 31/4406

(52) U.S. Cl. CPC A61K 31/4406
USPC 514/357

(57) ABSTRACT

Methods of treating patients with SNDX-275 are provided.

### CROSS-REFERENCE

This application is a continuation of U.S. application Ser. No. 12/743,557, filed on Aug. 9, 2010, which claims priority to International Application No. PCT/US2008/084009, filed with the U.S. Receiving Office on Nov. 19, 2008, which claims priority to U.S. Provisional Application No. 60/989,082, filed on Nov. 19, 2007, each of which is incorporated by reference in its entirety.

### BACKGROUND OF THE INVENTION

DNA in eukaryotic cells is tightly complexed with proteins to form chromatin. Histones are small proteins that are tightly complexed with DNA to form a nucleosome, which is further connected by linker DNA to form a solenoid. Histones extending from the nucleosomal core are enzymatically modified, affecting chromatin structure and gene expression. The study of inhibitors of histone deacetylases (HDACs) indicates that these enzymes play an important role in cell proliferation and differentiation. The apparent involvement of HDACs in the control of cell proliferation and differentiation suggests that aberrant HDAC activity may play a role in cancer.

Histone hyperacetylation by HDAC inhibition neutralizes the positive charge of the lysine side chain, and is associated with change of the chromatin structure and the consequential transcriptional activation of a number of genes. It is believed that one outcome of histone hyperacetylation is induction of the Cyclin-dependent kinase inhibitory protein, P21, which causes cell cycle arrest. HDAC inhibitors such as Trichostatin A (TSA) and suberoylanilide hydroxamic acid (SAHA) have been reported to inhibit cell growth, induce terminal differentiation in tumor cells and prevent the formation of tumors in mice. HDACs have been viewed as attractive targets for anticancer drug development with their ability to block angiogenesis and cell cycling, and promote apoptosis and differentiation.

Compounds and compositions capable of inhibiting histone deacetylating enzymes and inducing differentiation are useful as therapeutic or ameliorating agents for diseases that are involved in cellular growth such as malignant tumors, autoimmune diseases, skin diseases, infections, other anti-proliferative therapies, etc. HDAC inhibitors are able to target the transcription of specific disease-causing genes as well as improve the efficacy of existing cytostatics (such as the retinoids). Due to its role in the transcriptional mechanism to affect the gene expression, HDAC inhibitors are also useful as a therapeutic or prophylactic agent for diseases caused by abnormal gene expression such as inflammatory disorders, diabetes, diabetic complications, homozygous thalassemia, fibrosis, cirrhosis, acute promyelocytic leukemia (APL), organ transplant rejections, autoimmune diseases, protozoal infections, tumors, etc.

### Selective HDAC Background

N-(2-aminophenyl) 4-[N-(pyrid-3-yl)methyloxycarbonylaminomethyl]benzamide (also 3-pyridylmethyl-N-{4-[(2-aminophenyl)-carbamoyl]-benzyl}carbamate) is a compound of the formula I, below, having selective histone deacetylase inhibitory activity.

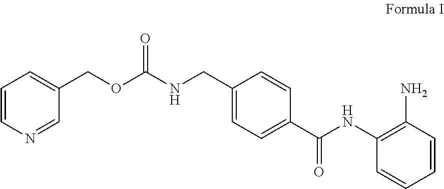 [SMILES: Nc1ccccc1NC(=O)c1ccc(CNC(=O)OCc2cccnc2)cc1]

The compound of formula I is hereinafter referred to as SNDX-275, which has been shown in pre-clinical in vitro and in vivo to be both an inhibitor of HDAC and a potent antitumor agent.

### SUMMARY OF THE INVENTION

The inventors have identified a need for methods of dosing SNDX-275. The present invention meets this need and provides related advantages as well.

Embodiments disclosed herein meet the foregoing needs and provide related advantages by providing a method of treating cancer in a patient, comprising administering to the patient a first dose of 10 mg SNDX-275 during a first biweek of a biweekly dosing schedule and a second dose of 10 mg of SNDX-275 during a second biweek of the biweekly dosing cycle, wherein the biweekly dosing schedule comprises at least two consecutive biweeks. In some embodiments, the first dose of SNDX-275 is administered on day 1 to day 4 of the first biweek and the second dose of SNDX-275 is administered on day 1 to day 4 of the second biweek. In some embodiments, the first dose of SNDX-275 is administered on day 1 to day 3 of the first biweek and the second dose of SNDX-275 is administered on day 1 to day 3 of the second biweek. In some embodiments, the first dose of SNDX-275 is administered on day 1 of the first biweek and the second dose of SNDX-275 is administered on day 1 of the second biweek. In some embodiments, the method further comprises administering to the patient at least one 5 mg dose of SNDX-275 after the end of the biweekly dosing cycle schedule. In some embodiments, the method further comprises detecting a drug-related toxicity in the patient and subsequently administering to the patient a reduced dose of SNDX-275. In some embodiments, the reduced dose is 5 mg of SNDX-275 per dose. In some embodiments, the reduced dose is administered to the patient on a biweekly dosing schedule, wherein a first dose of 5 mg of SNDX-275 is administered to the patient during the first biweek and a second dose of 5 mg of SNDX-275 is administered to the patient during the second biweek. In some embodiments, the first dose of SNDX-275 is administered on day 1 to day 4 of the first biweek and the second dose of SNDX-275 is administered on day 1 to day 4 of the second biweek. In some embodiments, the first dose of SNDX-275 is administered on day 1 to day 3 of the first biweek and the second dose of SNDX-275 is administered on day 1 to day 3 of the second biweek. In some embodiments, the first dose of SNDX-275 is administered on day 1 of the first biweek and the second dose of SNDX-275 is administered on day 1 of the second biweek. In some embodiments, SNDX-275 is administered orally. In some embodiments, SNDX-275 is administered orally in the form of one or more tablets. In some embodiments, SNDX-275 is administered orally in the form of 0.5, 1, 2, 3, 4, 5, 6, 7, 8, 9 or 10 mg tablets or a suitable combination of 2 or more thereof.

Some embodiments meet the foregoing and additional needs by providing a method of treating cancer in a patient, comprising administering to the patient at least one dose of 10 mg of SNDX-275 and at least one subsequent dose of 5 mg of SNDX-275. In some embodiments, the method further comprises, after administering the 10 mg of SNDX-275 to the patient, detecting a drug-related toxicity in the patient, and subsequently administering the 5 mg dose of SNDX-275 to the patient. In some embodiments, the 10 mg dose of SNDX-275 is administered as part of a biweekly dosing schedule, wherein a first dose of 10 mg is administered during a first biweek and optionally a second dose of 10 mg is administered during a second biweek. In some embodiments, the 10 mg dose of SNDX-275 is administered as part of a biweekly dosing schedule, wherein a first dose of 10 mg of SNDX-275 is administered during the first biweek, a drug-related toxicity is then detected, and a second dose of 5 mg of SNDX-275 is administered during the second biweek. In some embodiments, the mean area under the plasma concentration curve of SNDX-275 is about 100 ng·h/mL to about 400 ng·h/mL. In some embodiments, the mean maximum plasma concentration of SNDX-275 is about 1 to about 60 ng/mL. In some embodiments, SNDX-275 is administered orally. In some embodiments, SNDX-275 is administered orally in the form of one or more tablets. In some embodiments, SNDX-275 is administered orally in the form of 0.5, 1, 2, 3, 4, 5, 6, 7, 8, 9 or 10 mg tablets or a suitable combination of 2 or more thereof.

Some embodiments meet the foregoing needs and provide related advantages by providing a method of treating cancer in a patient, comprising administering to the patient a first dose of 5 mg SNDX-275 during a first biweek of a biweekly dosing schedule and a second dose of 5 mg of SNDX-275 during a second biweek of the biweekly dosing cycle, wherein the biweekly dosing schedule comprises at least two consecutive biweeks. In some embodiments, the first dose of SNDX-275 is administered on day 1 to day 4 of the first biweek and the second dose of SNDX-275 is administered on day 1 to day 4 of the second biweek. In some embodiments, the first dose of SNDX-275 is administered on day 1 to day 3 of the first biweek and the second dose of SNDX-275 is administered on day 1 to day 3 of the second biweek. In some embodiments, the first dose of SNDX-275 is administered on day 1 of the first biweek and the second dose of SNDX-275 is administered on day 1 of the second biweek. In some embodiments, the mean area under the plasma concentration curve of SNDX-275 is about 150 ng·h/mL to about 350 ng·h/mL. In some embodiments, the mean maximum plasma concentration of SNDX-275 is about 1 to about 50 ng/mL. In some embodiments, SNDX-275 is administered orally. In some embodiments, SNDX-275 is administered orally in the form of one or more tablets. In some embodiments, SNDX-275 is administered orally in the form of 0.5, 1, 2, 3, 4, 5, 6, 7, 8, 9 or 10 mg tablets or a suitable combination of 2 or more thereof.

Some embodiments meet the foregoing and additional needs by providing a method of treating cancer in a patient, comprising administering to the patient a first dose of 7 mg SNDX-275 during a first biweek of a biweekly dosing schedule and a second dose of 7 mg of SNDX-275 during a second biweek of the biweekly dosing cycle, wherein the biweekly dosing schedule comprises at least two consecutive biweeks. In some embodiments, the first dose of SNDX-275 is administered on day 1 to day 4 of the first biweek and the second dose of SNDX-275 is administered on day 1 to day 4 of the second biweek. In some embodiments, the first dose of SNDX-275 is administered on day 1 to day 3 of the first biweek and the second dose of SNDX-275 is administered on day 1 to day 3 of the second biweek. In some embodiments, the first dose of SNDX-275 is administered on day 1 of the first biweek and the second dose of SNDX-275 is administered on day 1 of the second biweek. In some embodiments, the mean area under the plasma concentration curve of SNDX-275 is about 100 ng·h/mL to about 400 ng·h/mL. In some embodiments, the mean maximum plasma concentration of SNDX-275 is about 1 to about 60 ng/mL. In some embodiments, SNDX-275 is administered orally. In some embodiments, SNDX-275 is administered orally in the form of one or more tablets. In some embodiments, SNDX-275 is administered orally in the form of 0.5, 1, 2, 3, 4, 5, 6, 7, 8, 9 or 10 mg tablets or a suitable combination of 2 or more thereof.

The foregoing and additional needs are met by embodiments that provide a method of treating cancer in a patient, comprising administering to the patient a first dose of 3 mg SNDX-275 during a first biweek of a biweekly dosing schedule and a second dose of 3 mg of SNDX-275 during a second biweek of the biweekly dosing cycle, wherein the biweekly dosing schedule comprises at least two consecutive biweeks. In some embodiments, the first dose of SNDX-275 is administered on day 1 to day 4 of the first biweek and the second dose of SNDX-275 is administered on day 1 to day 4 of the second biweek. In some embodiments, the first dose of SNDX-275 is administered on day 1 to day 3 of the first biweek and the second dose of SNDX-275 is administered on day 1 to day 3 of the second biweek. In some embodiments, the first dose of SNDX-275 is administered on day 1 of the first biweek and the second dose of SNDX-275 is administered on day 1 of the second biweek. In some embodiments, the mean area under the plasma concentration curve of SNDX-275 is about 100 ng·h/mL to about 350 ng·h/mL. In some embodiments, the mean maximum plasma concentration of SNDX-275 is about 1 to about 50 ng/mL. In some embodiments, SNDX-275 is administered orally. In some embodiments, SNDX-275 is administered orally in the form of one or more tablets. In some embodiments, SNDX-275 is administered orally in the form of 0.5, 1, 2, 3, 4, 5, 6, 7, 8, 9 or 10 mg tablets or a suitable combination of 2 or more thereof.

The foregoing and additional needs are met by embodiments that provide a method of treating cancer in patient, comprising administering a first dose of from 2 to 6 mg/m² of SNDX-275 on a first day of an at least 28-day dosing cycle, a second dose of from 2 to 6 mg/m² of SNDX-275 on a second day of the at least 28-day dosing cycle and a third dose of from 2 to 6 mg/m² on a third day of the at least 28-day dosing cycle. In some embodiments, the first dose of SNDX-275 is 2 mg/m². In some embodiments, the second dose of SNDX-275 and the third dose of SNDX-275 are each 2 mg/m². In some embodiments, the first dose of SNDX-275 is 4 mg/m². In some embodiments, the second dose of SNDX-275 and the third dose of SNDX-275 are each 4 mg/m². In some embodiments, the first dose of SNDX-275 is 6 mg/m². In some embodiments, the second dose of SNDX-275 and the third dose of SNDX-275 are each 6 mg/m². In some embodiments, the first dose of SNDX-275 is administered on day 1 to day 7 of the at least 28-day dosing cycle and the second dose of SNDX-275 and the third dose of SNDX-275 are each administered on day 8 to day 28 of the at least 28-day dosing cycle. In some embodiments, the first dose of SNDX-275 is administered on day 1 to day 7 of the at least 28-day dosing cycle and the second dose of SNDX-275 and the third dose of SNDX-275 are each administered on day 8 to day 21 of the at least 28-day dosing cycle. In some embodiments, the first dose of SNDX-275 is administered on day 1 to day 4 of the at least 28-day dosing cycle, the second dose of SNDX-275 is administered on day 8 to day 11 of the at least 28-day dosing cycle and the third dose of SNDX-275 is administered on day 15 to day 18 of the at least 28-day dosing cycle. In some embodiments, the first dose of SNDX-275 is administered on day 1 to day 3 of the at least 28-day dosing cycle, the second dose of SNDX-275 is administered on day 8 to day 10 of the at least 28-day dosing cycle and the third dose of SNDX-275 is administered on day 15 to day 17 of the at least 28-day dosing cycle. In some embodiments, the first dose of SNDX-275 is administered on day 1 of the at least 28-day dosing cycle, the second dose of SNDX-275 is administered on day 8 of the at least 28-day dosing cycle and the third dose of SNDX-275 is administered on day 15 of the at least 28-day dosing cycle. In some embodiments, the mean area under the plasma concentration curve of SNDX-275 is about 100 ng·h/mL to about 350 ng·h/mL. In some embodiments, the mean maximum plasma concentration of SNDX-275 is about 1 to about 50 ng/mL. In some embodiments, SNDX-275 is administered orally. In some embodiments, SNDX-275 is administered orally in the form of one or more tablets. In some embodiments, SNDX-275 is administered orally in the form of 0.5, 1, 2, 3, 4, 5, 6, 7, 8, 9 or 10 mg tablets or a suitable combination of 2 or more thereof.

Some embodiments provided herein meet the foregoing and additional needs by providing a method of treating cancer in a patient, comprising administering to the patient two doses of about 2 to about 10 mg/m² each of SNDX-275 over the course of a 4 week treatment cycle, wherein a first dose of SNDX-275 is administered during week 1, a second dose of SNDX-275 is administered during week 2, and no dose of SNDX-275 is administered during each of weeks 3 and 4. In some embodiments, the first dose is about 2 mg/m². In some embodiments, the second dose is about 2 mg/m². In some embodiments, the first dose is about 4 mg/m². In some embodiments, the second dose is about 4 mg/m². In some embodiments, the first dose is about 6 mg/m². In some embodiments, the second dose is about 6 mg/m². In some embodiments, the second dose is about 8 mg/m². In some embodiments, the second dose is about 8 mg/m². In some embodiments, the mean area under the plasma concentration curve of SNDX-275 is about 150 ng·h/mL to about 350 ng·h/mL. In some embodiments, the mean maximum plasma concentration of SNDX-275 is about 1 to about 50 ng/mL. In some embodiments, the mean time to maximum plasma concentration of SNDX-275 is about 1.5 to about 6 hours. In some embodiments, SNDX-275 is administered orally. In some embodiments, SNDX-275 is administered orally in the form of one or more tablets. In some embodiments, SNDX-275 is administered orally in the form of 0.5, 1, 2, 3, 4, 5, 6, 7, 8, 9 or 10 mg tablets or a suitable combination of 2 or more thereof.

Some embodiments herein provide a method of treating cancer in a patient, comprising administering to the patient four doses of about 2 to about 10 mg/m² each of SNDX-275 over the course of a 6 week treatment cycle, wherein a first dose of SNDX-275 is administered during week 1, a second dose of SNDX-275 is administered during week 2, a third dose of SNDX-275 is administered during week 3, a fourth dose is administered during week 4, and no dose of SNDX-275 is administered during each of weeks 5 and 6. In some embodiments, the first dose is about 2 mg/m². In some embodiments, each of the second, third and fourth doses is about 2 mg /m². In some embodiments, the first dose is about 4 mg/m². In some embodiments, each of the second, third and fourth doses is about 4 mg/m². In some embodiments, the first dose is about 6 mg/m². In some embodiments, each of the second, third and fourth doses is about 6 mg/m². In some embodiments, the first dose is about 8 mg/m². In some embodiments, each of the second, third and fourth doses is about 8 mg/m². In some embodiments, the second dose is about 10 mg/m². In some embodiments, each of the second, third and fourth doses is about 10 mg/m². In some embodiments, the mean area under the plasma concentration curve of SNDX-275 is about 300 ng·h/mL to about 350 ng·h/mL. In some embodiments, the mean maximum plasma concentration of SNDX-275 is about 40 to about 60 ng/mL. In some embodiments, the mean time to maximum plasma concentration of SNDX-275 is about 0.5 to about 6 hours. In some embodiments, SNDX-275 is administered orally. In some embodiments, SNDX-275 is administered orally in the form of one or more tablets. In some embodiments, SNDX-275 is administered orally in the form of 0.5, 1, 2, 3, 4, 5, 6, 7, 8, 9 or 10 mg tablets or a suitable combination of 2 or more thereof.

Some embodiments provide a method of treating cancer in a patient, comprising administering a first dose of a composition comprising 2-10 mg/m² of SNDX-275 on day 1 and administering a second dose of a composition comprising 2-10 mg/m² of SNDX-275 between day 8 and 29. In some embodiments, the SNDX-275 in said composition has a half-life of greater than about 24 hours.

Some embodiments provide a method of treating cancer in a patient, comprising administering a composition comprising 2-6 mg/m² of SNDX-275 to the patient. In some embodiments, said administration is oral.

Some embodiments provide a method of treating cancer in a patient, comprising administering to said patient a composition comprising SNDX-275 under such conditions and in sufficient amount to give rise to a Cₘₐₓ for SNDX-275 of from about 1 to about 5 ng/mL. In some embodiments, said administration is oral.

Some embodiments provide a method of treating cancer in a patient, comprising administering to a patient a composition comprising SNDX-275, wherein said composition produces a Cₘₐₓ of SNDX-275 in the patient of between 10 and 100 ng/mL. In some embodiments, the method comprises administering 6-10 mg/m² of SNDX-275 to the patient. In some embodiments, said administration is oral.

Some embodiments provide a method of treating cancer in a patient, comprising administering a composition comprising SNDX-275 to the patient, wherein said composition gives rise to an SNDX-275 AUC of about 80-210 ng·h/mL. In some embodiments, the administered composition contains 4-10 mg/m² of SNDX-275.

Some embodiments provide a method of treating cancer in a patient, comprising administering a first dose of a composition comprising 10-100 mg/kg of SNDX-275 on day 1 and administering a second dose of a composition comprising 10-100 mg/kg of SNDX-275 between day 8 and 29. In some embodiments, the SNDX-275 in said composition has a half-life of greater than about 24 hours.

Thus, some embodiments provide a method of treating cancer in a patient, comprising administering to the patient a first dose of SNDX-275, wherein the dose of SNDX-275 produces in the patient an area under the plasma concentration curve (AUC) for SNDX-275 in the range of about 100 to about 400 ng·h/mL. In some embodiments, a Cmax of about 2.0 to about 50 ng/mL of SNDX-275 is achieved in the patient. In some embodiments, a Cmax is obtained within 3-36 hours of administering the SNDX-275 to the patient. In some embodiments, the mean Cmax across a patient population is in the range of about 4 to about 40 ng/mL. In some embodiments, the method further comprises administering a second dose of SNDX-275 to the patient. In some embodiments, the first dose is administered on day 1 and the second dose is administered on one of days 4-16. In some embodiments, the method further comprises administering a third dose of SNDX-275 to the patient. In some embodiments, the first dose is administered on day 1, the second dose on day 4-16 and the third dose on day 14-24. In some embodiments, the dose of SNDX-275 has a T_{1/2} of from about 20 to about 60 hours. In some embodiments, T_{1/2} for SNDX-275 is about 30 to about 50 hours. In some embodiments, the patient has a hematologic malignancy, a solid tumor or a lymphoma. In some embodiments, the patient has a hematologic malignancy. In some embodiments, the first dose of SNDX-275 contains no more than 7 mg/m² of SNDX-275. In some embodiments, the first dose of SNDX-275 contains no more than 6 mg/m² of SNDX-275. In some embodiments, the first dose of SNDX-275 contains from about 0.1 to about 6 mg/m² of SNDX-275. In some embodiments, the first dose is administered orally. In some embodiments, each dose is administered orally.

Some embodiments provide methods of treating cancer in a patient, comprising administering to the patient a fixed dose of about 1 mg to about 10 mg of SNDX-275 no more than one time per week. In some embodiments, the fixed dose is about 1 mg, 2 mg, 3 mg, 4 mg, 5 mg, 6 mg, 7 mg, 8 mg, 9 mg or 10 mg of SNDX-275, administered one time per week. In some embodiments, the fixed dose is about 1 mg to about 6 mg of SNDX-275, administered no more than one time per week. In some embodiments, the fixed dose is about 1 mg, 2 mg, 3 mg, 4 mg, 5 mg or 6 mg of SNDX-275, administered no more than one time per week. In some embodiments, the amount of SNDX-275 administered is sufficient to give rise to certain PK parameters in the patient. In some embodiments, the mean area under the plasma concentration curve of SNDX-275 is about 1 ng·h/mL to about 400 ng·h/mL. In some embodiments, the mean maximum plasma concentration of SNDX-275 is about 40 to about 60 ng/mL. In some embodiments, the mean time to maximum plasma concentration of SNDX-275 is about 0.5 to about 24 hours. In some embodiments, the SNDX-275 is administered orally. In some embodiments, the SNDX-275 is administered orally in the form of one or more tablets. In some embodiments, the SNDX-275 is administered orally in the form of 0.5, 1, 2, 3, 4, 5, 6, 7, 8, 9 or 10 mg tablets or a suitable combination of 2 or more thereof.

Some embodiments provide a method of treating cancer in a patient, comprising administering to the patient a fixed dose of about 1 mg to about 10 mg of SNDX-275 one time every other week. In some embodiments, the fixed dose is about 1 mg, 2 mg, 3 mg, 4 mg, 5 mg, 6 mg, 7 mg, 8 mg, 9 mg or 10 mg of SNDX-275, administered one time every other week. In some embodiments, the fixed dose is about 1 mg to about 6 mg of SNDX-275, administered one time every other week. In some embodiments, the fixed dose is about 1 mg, 2 mg, 3 mg, 4 mg, 5 mg or 6 mg of SNDX-275, administered one time every other week. In some embodiments, the amount of SNDX-275 administered is sufficient to give rise to certain PK parameters in the patient. In some embodiments, the mean area under the plasma concentration curve of SNDX-275 is about 1 ng·h/mL to about 400 ng·h/mL. In some embodiments, the mean maximum plasma concentration of SNDX-275 is about 40 to about 60 ng/mL. In some embodiments, the mean time to maximum plasma concentration of SNDX-275 is about 0.5 to about 24 hours. In some embodiments, the SNDX-275 is administered orally. In some embodiments, the SNDX-275 is administered orally in the form of one or more tablets. In some embodiments, the SNDX-275 is administered orally in the form of 0.5, 1, 2, 3, 4, 5, 6, 7, 8, 9 or 10 mg tablets or a suitable combination of 2 or more thereof.

In some embodiments, the SNDX-275 is administered orally to a cancer patient in an amount of about 4-8 mg/m² and the SNDX-275 causes a dose-dependent induction of marrow blast apoptosis. In some embodiments the SNDX-275 is administered orally to a cancer patient in an amount of about 4 mg/m² In some embodiments the SNDX-275 is administered orally to a cancer patient in an amount of about 6 mg/m². In some embodiments the SNDX-275 is administered orally to a cancer patient in an amount of about 8 mg/m².

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts time-course plasma concentration curves for SNDX-275 when dosed orally at dosages of 2, 4 and 6 mg/m² SNDX-275 on a biweekly schedule

FIG. 2 depicts time-course plasma concentration curves for SNDX-275 when dosed orally at dosages of 2, 4 and 5 mg/m² SNDX-275 on a 2×weekly schedule.

FIG. 3 depicts AUC versus dose for the curves depicted in FIG. 1 (3a) and FIG. 2 (3b), respectively. It can be seen that AUC is dose dependent, although there is variance in the AUC from patient-to-patient.

FIG. 4 shows time-course plasma concentration curves for SNDX-275 when dosed orally at dosages of 2, 4, 6, 8, 10 and 12 mg/m² SNDX-275.

FIG. 5 shows AUC versus dose for the data points depicted in FIG. 4.

FIG. 6 shows that at a mean AUC of about 220 ng·h/mL no dose limiting toxicity is observed, whereas dose-limiting toxicity appears to be associated with a mean AUC of about 600 ng·h/mL.

FIG. 7 depicts the time course plasma concentration curves for SNDX-275 when dosed orally of dosages of 4, 6, 8 and 10 mg/m² (7A) and AUC versus dose (7B) for the same data points.

### DETAILED DESCRIPTION OF THE INVENTION

Provided herein is a method of treating a disease state, in particular cancer, by administering to a patient in need of such treatment an effective dose of the HDAC inhibitor SNDX-275. In some embodiments, the cancer is a solid tumor; in others it is a leukemia. In particular embodiments, the mode of administration is oral administration. In some embodiments, about 0.5 to about 10 mg of SNDX-275 are administered to the patient. In some embodiments, about 1 to about 8, about 2 to about 6, about 2, about 4, about 6 or about 8 mg of SNDX-275 are administered to the patient, especially where such administration is oral administration. In some embodiments, the administration may be repeated, e.g. on a twice weekly (2×weekly, semiweekly) schedule, a weekly schedule, a biweekly schedule, a monthly schedule, etc. In some embodiments, SNDX-275 is administered on a weekly schedule for 1, 2, 3, 4, 5, 6 or more weeks. In some embodiments, SNDX-275 is administered on a weekly schedule for 1, 2, 3, 4, 5 or 6 or more weeks, followed by a period in which no SNDX-275 is administered (wash-out period), which may be 1, 2, 3, 4 or more weeks. In some embodiments, the wash-out period is from about 1 day to about 3 weeks, or about 3 days to about 1 week, or about 1 week to about 2 weeks, or about 2 weeks to about 3 weeks. In some embodiments, SNDX-275 is administered weekly for 2 weeks, followed by a 1, 2 or 3 week wash-out period. In some embodiments, SNDX-275 is administered weekly for 3 weeks, followed by a 1, 2 or 3 week wash-out period. In some embodiments, SNDX-275 is administered weekly for 4 weeks, followed by a 1, 2 or 3 week wash-out period. In some embodiments, SNDX-275 is administered on a weekly schedule for 1, 2, 3, 4, 5, 6 or more weeks. In some embodiments, SNDX-275 is administered on a 2×weekly schedule for 1, 2, 3, 4, 5 or 6 or more weeks, followed by a period in which no SNDX-275 is administered (wash-out period), which may be 1, 2, 3, 4 or more weeks. In some embodiments, SNDX-275 is administered 2×weekly for 2 weeks, followed by a 1, 2 or 3 week wash-out period. In some embodiments, SNDX-275 is administered 2×weekly for 3 weeks, followed by a 1, 2 or 3 week wash-out period. In some embodiments, SNDX-275 is administered 2×weekly for 4 weeks, followed by a 1, 2 or 3 week wash-out period. In some embodiments, SNDX-275 is administered on a biweekly schedule. In some embodiments, biweekly dosing is repeated 1, 2, 3, 4, 5, 6 or more times, followed by a period of wash-out. In some embodiments, SNDX-275 is administered on a biweekly schedule for 1, 2, 3, 4, 5 or 6 or more biweeks, followed by a wash-out period of 1, 2, 3, 4 or more weeks. In some embodiments, SNDX-275 is administered biweekly for 2 biweeks, followed by a 1, 2 or 3 week wash-out period. In some embodiments, SNDX-275 is administered biweekly for 3 biweeks, followed by a 1, 2 or 3 week wash-out period. In some embodiments, SNDX-275 is administered weekly for 4 biweeks, followed by a 1, 2 or 3 week wash-out period. In some embodiments, SNDX-275 is administered on a biweekly schedule for 1, 2, 3, 4, 5, 6 or more biweeks. In some embodiments, the administered SNDX-275 produces an area under the plasma concentration curve (AUC) in the patient of about 100 to about 800 ng·h/mL. In some embodiments, the Cmax for SNDX-275 is about 1 to about 100 ng/mL. In some embodiments, Tmax is achieved from 0.5 to 24 hours after administration of SNDX-275.

In some embodiments, SNDX-275 is administered orally in a dosage range of about 2 to about 10, about 2 to about 8 or about 2 to about 6 mg/m². In some embodiments, SNDX-275 is administered to the patient orally at a dosage of about 2, about 4, about 5 or about 6 mg/m². At these dosages, SNDX-275 is administered less frequently than once per day. In some embodiments, the SNDX-275 is administered less frequently than once per week. In some embodiments, the SNDX-275 is administered orally twice per week for at least a week. In some embodiments, SNDX-275 is administered once per week for at least two weeks. In some embodiments, SNDX-275 is administered at least twice—every other week. In some embodiments, the administered SNDX-275 produces an area under the plasma concentration curve (AUC) in the patient of about 100 to about 800 ng·h/mL. In some embodiments, the Cmax for SNDX-275 is about 1 to about 100 ng/mL. In some embodiments, Tmax is achieved from 0.5 to 24 hours after administration of SNDX-275. The treated patient is generally suffering from cancer—e.g. a solid tumor cancer or a leukemia.

In some embodiments, SNDX-275 is administered orally to a cancer patient. The cancer may be either a solid tumor or a leukemia. In some embodiments, the administration occurs on a cycle comprising a dosing period and a wash-out period. In some embodiments, the dosing period is biweekly, weekly or 2×weekly. In some embodiments, the oral dose administered is about 1 to 10, about 2 to 8 or about 2 to 6 mg/m² of SNDX-275. In some embodiments, the oral dose is 2, 4, 5, 6, 8 or 10 mg/m² of SNDX-275. In some embodiments, the oral dose of SNDX-275 is 2, 4, 6, 8 or 10 mg/m² of SNDX-275 administered on a 2×weekly schedule, after which the cycle may be repeated. In some embodiments, the oral dose of SNDX-275 administered is 2 mg/m² administered on a 2×weekly schedule, after which the cycle may be repeated. In some embodiments, the oral dose of SNDX-275 administered is 2, 4, 6, 8 or 10 mg/m² on a 2×weekly schedule for 1, 2, 3, 4, 5 or 6 weeks, followed by a 1, 2, 3 or 4 week washout period, after which the cycle may be repeated. In some embodiments, the oral dose of SNDX-275 administered is 2 mg/m² on a 2×weekly schedule for 1, 2, 3, 4, 5 or 6 weeks, followed by a 1, 2, 3 or 4 week washout period, after which the cycle may be repeated. In some embodiments, the oral dose of SNDX-275 administered is 2, 4, 5, 6, 8 or 10 mg/m² of SNDX-275 on a weekly schedule for 1, 2, 3, 4, 5 or 6 weeks, followed by a 1, 2, 3 or 4 week washout period, after which the cycle may be repeated. In some embodiments, the oral dose of SNDX-275 administered is 2 mg/m², 4 mg/m2 or 5 mg/m² on a weekly schedule for 1, 2, 3, 4, 5 or 6 weeks, followed by a 1, 2, 3 or 4 week washout period, after which the cycle may be repeated. In some embodiments, the oral dose of SNDX-275 administered is 2, 4, 5, 6, 8 or 10 mg/m² on a biweekly schedule of about 1, 2, 3, 4, 5 or 6 biweeks, followed by a wash-out period of about 1, 2, 3 or 4 weeks, after which the cycle may be repeated. In some embodiments, the oral dose of SNDX-275 administered is 2, 4, 5 or 6 mg/m² on a biweekly schedule of about 1, 2, 3, 4, 5 or 6 biweeks, followed by a wash-out period of about 1, 2, 3 or 4 weeks, after which the cycle may be repeated.

### Certain Terminology

Unless defined otherwise, all technical and scientific terms used herein have the same meaning as is commonly understood by one of skill in the art to which the claimed subject matter belongs. In the event that there is a plurality of definitions for terms herein, those in this section prevail. Where reference is made to a URL or other such identifier or address, it is understood that such identifiers can change and particular information on the internet can come and go, but equivalent information can be found by searching the internet or other appropriate reference source. Reference thereto evidences the availability and public dissemination of such information.

It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a”, “an” and “the” include plural referents unless the context clearly dictates otherwise. It should also be noted that use of “or” means “and/or” unless stated otherwise. Furthermore, use of the term “including” as well as other forms, such as “include”, “includes”, and “included” is not limiting.

Definition of standard chemistry terms may be found in reference works, including Carey and Sundberg “ADVANCED ORGANIC CHEMISTRY 4^{TH} ED.” Vols. A (2000) and B (2001), Plenum Press, New York. Unless otherwise indicated, conventional methods of mass spectroscopy, NMR, HPLC, IR and UV/Vis spectroscopy and pharmacology, within the skill of the art are employed. Unless specific definitions are provided, the nomenclature employed in connection with, and the laboratory procedures and techniques of, analytical chemistry, synthetic organic chemistry, and medicinal and pharmaceutical chemistry described herein are those known in the art. Standard techniques can be used for chemical syntheses, chemical analyses, pharmaceutical preparation, formulation, and delivery, and treatment of patients. Reactions and purification techniques can be performed e.g., using kits of manufacturer's specifications or as commonly accomplished in the art or as described herein. The foregoing techniques and procedures can be generally performed of conventional methods well known in the art and as described in various general and more specific references that are cited and discussed throughout the present specification. Throughout the specification, groups and substituents thereof can be chosen by one skilled in the field to provide stable moieties and compounds.

The compounds presented herein may exist as tautomers. Tautomers are compounds that are interconvertible by migration of a hydrogen atom, accompanied by a switch of a single bond and adjacent double bond. In solutions where tautomerization is possible, a chemical equilibrium of the tautomers will exist. The exact ratio of the tautomers depends on several factors, including temperature, solvent, and pH. Some examples of tautomeric pairs include:

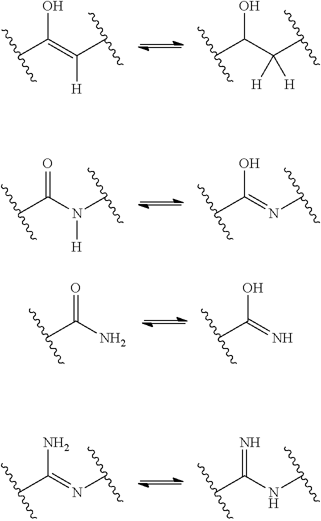

The HDACs are a family including at least eighteen enzymes, grouped in three classes (Class I, II and III). Class I HDACs include, but are not limited to, HDACs 1, 2, 3, and 8. Class I HDACs can be found in the nucleus and are believed to be involved with transcriptional control repressors. Class II HDACs include, but are not limited to, HDACS 4, 5, 6, 7, and 9 and can be found in both the cytoplasm as well as the nucleus. Class III HDACs are believed to be NAD dependent proteins and include, but are not limited to, members of the Sirtuin family of proteins. Non-limiting examples of sirtuin proteins include SIRT1-7. As used herein, the term “selective HDAC” refers to an HDAC inhibitor that does not significantly interact with all three HDAC classes. As used herein, a “Class I selective HDAC” refers to an HDAC inhibitor that interacts with one or more of HDACs 1, 2, 3 or 8, but does not significantly interact with the Class II HDACs (i.e., HDACs 4, 5, 6, 7 and 9).

The term “HDAC modulator” as used herein refers to a compound that has the ability to modulate transcriptional activity.

The term “HDAC inhibitor” as used herein refers to a compound that has the ability to reduce transcriptional activity. As a result, this therapeutic class is able to block angiogenesis and cell cycling, and promote apoptosis and differentiation. By targeting these key components of tumor proliferation, HDAC inhibitors have the potential as anticancer agents. HDAC inhibitors both display targeted anticancer activity by itself and improve the efficacy of existing agents as well as other new targeted therapies.

The term “subject”, “patient” or “individual” as used herein in reference to individuals suffering from a disorder, and the like, encompasses mammals and non-mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In some embodiments of the methods and compositions provided herein, the mammal is a human.

The terms “treat,” “treating” or “treatment,” and other grammatical equivalents as used herein, include alleviating, abating or ameliorating a disease or condition symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition, and are intended to include prophylaxis. The terms further include achieving a therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made.

As used herein, the terms “cancer treatment” “cancer therapy” and the like encompasses treatments such as surgery (such as cutting, abrading, ablating (by physical or chemical means or a combination of physical or chemical means), suturing, lasering or otherwise physically changing body tissues and organs), radiation therapy, administration of chemotherapeutic agents and combinations of any two or all of these methods. Combination treatments may occur sequentially or concurrently. Treatments(s), such as radiation therapy and/or chemotherapy, that is administered prior to surgery, is referred to as neoadjuvant therapy. Treatments(s), such as radiation therapy and/or chemotherapy, administered after surgery is referred to herein as adjuvant therapy.

Examples of surgeries that may be used for cancer treatment include, but are not limited to radical prostatectomy, cryotherapy, mastectomy, lumpectomy, transurethral resection of the prostate, and the like.

Many chemotherapeutic agents are known and may operate via a wide variety of modes of action. In some nonlimiting embodiments of the present invention, the chemotherapeutic agent is a cytotoxic agent, an antiproliferative, a targeting agent (such as kinase inhibitors and cell cycle regulators), or a biologic agent (such as cytokines, vaccines, viral agents, and other immunostimulants such as BCG, hormones, monocolonal antibodies and siRNA). The nature of a combination therapy involving administration of a chemotherapeutic agent will depend upon the type of agent being used.

SNDX-275 may be administered in combination with surgery, as an adjuvant, or as a neoadjuvant agent. SNDX-275 may be useful in instances where radiation and/or chemotherapy are indicated, to enhance the therapeutic benefit of these treatments, including induction chemotherapy, primary (neoadjuvant) chemotherapy, and both adjuvant radiation therapy and adjuvant chemotherapy. Radiation and chemotherapy frequently are indicated as adjuvants to surgery in the treatment of cancer. For example, radiation can be used both pre- and post-surgery as components of the treatment strategy for rectal carcinoma. SNDX-275 may be useful following surgery in the treatment of cancer in combination with radiation and/or chemotherapy.

Where combination treatments are contemplated, it is not intended that SNDX-275 be limited by the particular nature of the combination. For example, SNDX-275 may be administered in combination as simple mixtures as well as chemical hybrids. An example of the latter is where the compound is covalently linked to a targeting carrier or to an active pharmaceutical. Covalent binding can be accomplished in many ways, such as, though not limited to, the use of a commercially available cross-linking compound.

As used herein, the terms “pharmaceutical combination”, “administering an additional therapy”, “administering an additional therapeutic agent” and the like refer to a pharmaceutical therapy resulting from the mixing or combining of more than one active ingredient and includes both fixed and non-fixed combinations of the active ingredients. The term “fixed combination” means that SNDX-275, and at least one co-agent, are both administered to a patient simultaneously in the form of a single entity or dosage. The term “non-fixed combination” means that SNDX-275, and at least one co-agent, are administered to a patient as separate entities either simultaneously, concurrently or sequentially with variable intervening time limits, wherein such administration provides effective levels of the two or more compounds in the body of the patient. These also apply to cocktail therapies, e.g. the administration of three or more active ingredients.

As used herein, the terms “co-administration”, “administered in combination with” and their grammatical equivalents or the like are meant to encompass administration of the selected therapeutic agents to a single patient, and are intended to include treatment regimens in which the agents are administered by the same or different route of administration or at the same or different times. In some embodiments SNDX-275 will be co-administered with other agents. These terms encompass administration of two or more agents to an animal so that both agents and/or their metabolites are present in the animal at the same time. They include simultaneous administration in separate compositions, administration at different times in separate compositions, and/or administration in a composition in which both agents are present. Thus, in some embodiments, SNDX-275 and the other agent(s) are administered in a single composition. In some embodiments, SNDX-275 and the other agent(s) are admixed in the composition.

The terms “effective amount”, “therapeutically effective amount” or “pharmaceutically effective amount” as used herein, refer to a sufficient amount of at least one agent or compound being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising the compound as disclosed herein required to provide a clinically significant decrease in a disease. An appropriate “effective” amount in any individual case may be determined using techniques, such as a dose escalation study.

The terms “administer,” “administering”, “administration,” and the like, as used herein, refer to the methods that may be used to enable delivery of compounds or compositions to the desired site of biological action. These methods include, but are not limited to oral routes, intraduodenal routes, parenteral injection (including intravenous, subcutaneous, intraperitoneal, intramuscular, intravascular or infusion), topical and rectal administration. Those of skill in the art are familiar with administration techniques that can be employed with the compounds and methods described herein, e.g., as discussed in Goodman and Gilman, The Pharmacological Basis of Therapeutics, current ed.; Pergamon; and Remington's, Pharmaceutical Sciences (current edition), Mack Publishing Co., Easton, Pa. In preferred embodiments, the compounds and compositions described herein are administered orally.

The term “acceptable” as used herein, with respect to a formulation, composition or ingredient, means having no persistent detrimental effect on the general health of the subject being treated.

The term “pharmaceutically acceptable” as used herein, refers to a material, such as a carrier or diluent, which does not abrogate the biological activity or properties of SNDX-275, and is relatively nontoxic, i.e., the material may be administered to an individual without causing undesirable biological effects or interacting in a deleterious manner with any of the components of the composition in which it is contained.

The term “pharmaceutical composition,” as used herein, refers to a biologically active compound, optionally mixed with at least one pharmaceutically acceptable chemical component, such as, though not limited to carriers, stabilizers, diluents, dispersing agents, suspending agents, thickening agents, and/or excipients.

The term “carrier” as used herein, refers to relatively nontoxic chemical compounds or agents that facilitate the incorporation of the compound into cells or tissues.

The term “agonist,” as used herein, refers to a molecule such as the compound, a drug, an enzyme activator or a hormone modulator which enhances the activity of another molecule or the activity of a receptor site.

The term “antagonist,” as used herein, refers to a molecule such as the compound, a drug, an enzyme inhibitor, or a hormone modulator, which diminishes, or prevents the action of another molecule or the activity of a receptor site.

The term “modulate,” as used herein, means to interact with a target either directly or indirectly so as to alter the activity of the target, including, by way of example only, to enhance the activity of the target, to inhibit the activity of the target, to limit the activity of the target, or to extend the activity of the target.

The term “modulator,” as used herein, refers to a molecule that interacts with a target either directly or indirectly. The interactions include, but are not limited to, the interactions of an agonist and an antagonist.

The term “pharmaceutically acceptable derivative or prodrug” as used herein, refers to any pharmaceutically acceptable salt, ester, salt of an ester or other derivative of SNDX-275, which, upon administration to a recipient, is capable of providing, either directly or indirectly, a pharmaceutically active metabolite or residue thereof. Particularly favored derivatives or prodrugs are those that increase the bioavailability of the compounds of this invention when such compounds are administered to a patient (e.g., by allowing orally administered compound to be more readily absorbed into blood) or which enhance delivery of the parent compound to a biological compartment (e.g., the brain or lymphatic system).

The term “pharmaceutically acceptable salt” as used herein, refers to salts that retain the biological effectiveness of the free acids and bases of the specified compound and that are not biologically or otherwise undesirable. Compounds described herein may possess acidic or basic groups and therefore may react with any of a number of inorganic or organic bases, and inorganic and organic acids, to form a pharmaceutically acceptable salt. These salts can be prepared in situ during the final isolation and purification of the compounds of the invention, or by separately reacting a purified compound in its free base form with a suitable organic or inorganic acid, and isolating the salt thus formed. Examples of pharmaceutically acceptable salts include those salts prepared by reaction of SNDX-275 with a mineral or organic acid or an inorganic base, such salts including, acetate, acrylate, adipate, alginate, aspartate, benzoate, benzenesulfonate, bisulfate, bisulfite, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, γ-hydroxybutyrate, hydrochloride, hydrobromide, hydroiodide, 2-hydroxyethanesulfonate, iodide, isobutyrate, lactate, maleate, malonate, methanesulfonate, mandelate, metaphosphate, methanesulfonate, methoxybenzoate, methylbenzoate, monohydrogen phosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylate undeconate and xylenesulfonate. Other acids, such as oxalic, while not in themselves pharmaceutically acceptable, may be employed in the preparation of salts useful as intermediates in obtaining the compounds of the invention and their pharmaceutically acceptable acid addition salts. (See for example Berge et al., J. Pharm. Sci. 1977, 66, 1-19.) Further, those compounds described herein which may comprise a free acid group may react with a suitable base, such as the hydroxide, carbonate or bicarbonate of a pharmaceutically acceptable metal cation, with ammonia, or with a pharmaceutically acceptable organic primary, secondary or tertiary amine. Representative alkali or alkaline earth salts include the lithium, sodium, potassium, calcium, magnesium, and aluminum salts and the like. Illustrative examples of bases include sodium hydroxide, potassium hydroxide, choline hydroxide, sodium carbonate, N⁺(C₁₋₄ alkyl)₄, and the like. Representative organic amines useful for the formation of base addition salts include ethylamine, diethylamine, ethylenediamine, ethanolamine, diethanolamine, piperazine and the like. It should be understood that SNDX-275 also include the quaternization of any basic nitrogen-containing groups they may contain. Water or oil-soluble or dispersible products may be obtained by such quaternization. See, for example, Berge et al., supra.

The terms “enhance” or “enhancing,” as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system.

The term “metabolite,” as used herein, refers to a derivative of the compound which is formed when the compound is metabolized.

The term “active metabolite,” as used herein, refers to a biologically active derivative of the compound that is formed when the compound is metabolized.

The term “metabolized,” as used herein, refers to the sum of the processes (including, but not limited to, hydrolysis reactions and reactions catalyzed by enzymes) by which a particular substance is changed by an organism. Thus, enzymes may produce specific structural alterations to the compound. For example, cytochrome P450 catalyzes a variety of oxidative and reductive reactions while uridine diphosphate glucuronyltransferases catalyze the transfer of an activated glucuronic-acid molecule to aromatic alcohols, aliphatic alcohols, carboxylic acids, amines and free sulphydryl groups. Further information on metabolism may be obtained from The Pharmacological Basis of Therapeutics, 9th Edition, McGraw-Hill (1996).

Provided herein are pharmaceutical compositions SNDX-275 or a pharmaceutically acceptable salt, prodrug, solvate, polymorph, tautomer or isomer thereof. In various embodiments, the pharmaceutical composition comprises at least one pharmaceutically acceptable carrier.

Provided herein are methods for treating a patient suffering from a histone deacetylase mediated disorder, comprising administering to said individual an effective amount of a composition comprising SNDX-275 or a pharmaceutically acceptable salt, prodrug, solvate, polymorph, tautomer or isomer thereof. In some embodiments, SNDX-275 is administered in combination with an additional cancer therapy. In some embodiments, the additional cancer therapy is selected from surgery, radiation therapy, and administration of at least one chemotherapeutic agent. In various embodiments, the administration of SNDX-275 occurs after surgery. In other embodiments, the administration of SNDX-275 occurs before surgery. In some embodiments, the histone deacetylase mediated disorder is selected from the group consisting of inflammatory diseases, infections, autoimmune disorders, stroke, ischemia, cardiac disorder, neurological disorders, fibrogenetic disorders, proliferative disorders, hyperproliferative disorders, tumors, leukemias, neoplasms, cancers, carcinomas, metabolic diseases and malignant diseases. In other embodiments, the histone deacetylase mediated disorder is a hyperproliferative disease. In some embodiments, the histone deacetylase mediated disorder is cancer, tumors, leukemias, neoplasms, or carcinomas, including but not limited to cancer is brain cancer, breast cancer, lung cancer, ovarian cancer, pancreatic cancer, prostate cancer, renal cancer, colorectal cancer, leukemia, myeloid leukemia, glioblastoma, follicular lymphona, pre-B acute leukemia, chronic lymphocytic B-leukemia, mesothelioma or small cell line cancer. In yet other emobidments, the histone deacetylase mediated disorder is a proliferative disease selected from psoriasis, restenosis, autoimmune disease, or atherosclerosis.

Provided herein are methods for degrading, inhibiting the growth of or killing cancer cells comprising contacting the cells with an amount of a composition effective to degrade, inhibit the growth of or kill cancer cells, the composition comprising SNDX-275 or a pharmaceutically acceptable salt, prodrug, solvate, polymorph, tautomer or isomer thereof. In some embodiments, the cancer is brain cancer, breast cancer, lung cancer, ovarian cancer, pancreatic cancer, prostate cancer, renal cancer, colorectal cancer, leukemia, myeloid leukemia, glioblastoma, follicular lymphona, pre-B acute leukemia, chronic lymphocytic B-leukemia, mesothelioma or small cell line cancer. In some embodiments, the cancer cells comprise brain, breast, lung, ovarian, pancreatic, prostate, renal, or colorectal cancer cells.

Provided herein are methods of inhibiting tumor size increase, reducing the size of a tumor, reducing tumor proliferation or preventing tumor proliferation in an individual comprising administering to said individual an effective amount of a composition to inhibit tumor size increase, reduce the size of a tumor, reduce tumor proliferation or prevent tumor proliferation, the composition comprising SNDX-275 or a pharmaceutically acceptable salt, prodrug, solvate, polymorph, tautomer or isomer thereof. In some embodiments, the tumor occurs in the brain, breast, lung, ovaries, pancreas, prostate, kidney, colon or rectum. In some embodiments, SNDX-275 is administered in combination with an additional cancer therapy including, but not limited to surgery, radiation therapy, and administration of at least one chemotherapeutic agent. In some embodiments, the composition is administered before surgery. In other embodiments, the composition is administered after surgery.

SNDX-275, pharmaceutically acceptable salts, pharmaceutically active metabolites, pharmaceutically acceptable prodrugs, and pharmaceutically acceptable solvates thereof, may modulate the activity of HDAC enzymes; and, as such, is useful for treating diseases or conditions in which aberrant HDAC enzyme activity contributes to the pathology and/or symptoms of a disease or condition.

### Synthesis of SNDX-275

SNDX-275 may be obtained by synthesis as described in U.S. Pat. No. 6,174,905 (“US '905”), issued on Jan. 16, 2001. Specifically, the synthesis of SNDX-275 appear appearing at Example 48 of US '905 is incorporated by reference herein in its entirety.

### Pharmaceutically Acceptable Salts

SNDX-275 may also exist as its pharmaceutically acceptable salts, which may also be useful for treating disorders. For example, the invention provides for methods of treating diseases, by administering pharmaceutically acceptable salts of SNDX-275. The pharmaceutically acceptable salts can be administered as pharmaceutical compositions.

Thus, SNDX-275 can be prepared as pharmaceutically acceptable salts formed when an acidic proton present in the parent compound either is replaced by a metal ion, for example an alkali metal ion, an alkaline earth ion, or an aluminum ion; or coordinates with an organic base. Base addition salts can also be prepared by reacting the free acid form of SNDX-275 with a pharmaceutically acceptable inorganic or organic base, including, but not limited to organic bases such as ethanolamine, diethanolamine, triethanolamine, tromethamine, N-methylglucamine, and the like and inorganic bases such as aluminum hydroxide, calcium hydroxide, potassium hydroxide, sodium carbonate, sodium hydroxide, and the like. In addition, the salt forms of the disclosed compounds can be prepared using salts of the starting materials or intermediates.

Further, SNDX-275 can be prepared as pharmaceutically acceptable salts formed by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid, metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, p-toluenesulfonic acid, tartaric acid, trifluoroacetic acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, and muconic acid.

### Solvates

SNDX-275 may also exist in various solvated forms, which may also be useful for treating disorders. For example, the invention provides for methods of treating diseases, by administering solvates of SNDX-275. The solvates can be administered as pharmaceutical compositions. Preferably the solvates are pharmaceutically acceptable solvates.

Solvates contain either stoichiometric or non-stoichiometric amounts of a solvent, and may be formed during the process of crystallization with pharmaceutically acceptable solvents such as water, ethanol, and the like. Hydrates are formed when the solvent is water, or alcoholates are formed when the solvent is alcohol. Solvates of SNDX-275 can be conveniently prepared or formed during the processes described herein. By way of example only, hydrates of SNDX-275 can be conveniently prepared by recrystallization from an aqueous/organic solvent mixture, using organic solvents including, but not limited to, dioxane, tetrahydrofuran or methanol. In addition, the compounds provided herein can exist in unsolvated as well as solvated forms. In general, the solvated forms are considered equivalent to the unsolvated forms for the purposes of the compounds and methods provided herein.

### Polymorphs

SNDX-275 may also exist in various polymorphic states, all of which are herein contemplated, and which may also be useful for treating disorders. For example, the invention provides for methods of treating diseases, by administering polymorphs of SNDX-275. The various polymorphs can be administered as pharmaceutical compositions.

Thus, SNDX-275 include all crystalline forms, known as polymorphs. Polymorphs include the different crystal packing arrangements of the same elemental composition of the compound. Polymorphs may have different X-ray diffraction patterns, infrared spectra, melting points, density, hardness, crystal shape, optical and electrical properties, stability, solvates and solubility. Various factors such as the recrystallization solvent, rate of crystallization, and storage temperature may cause a single crystal form to dominate.

### Pharmaceutical Compositions

The present invention can be administered alone or as a pharmaceutical composition, thus the invention further provides pharmaceutical compositions and methods of making said pharmaceutical composition. In some embodiments, the pharmaceutical compositions comprise an effective amount of SNDX-275, or a pharmaceutically acceptable salt, prodrug, solvate, polymorph, tautomer or isomer thereof. The pharmaceutical composition may comprise of admixing at least one active ingredient, or a pharmaceutically acceptable salt, prodrug, solvate, polymorph, tautomer or isomer thereof, together with one or more carriers, excipients, buffers, adjuvants, stabilizers, or other materials well known to those skilled in the art and optionally other therapeutic agents. The formulations may conveniently be presented in unit dosage form and may be prepared by any methods well known in the art of pharmacy.

Examples of excipients that may be used in conjunction with the present invention include, but are not limited to water, saline, dextrose, glycerol or ethanol. The injectable compositions may also optionally comprise minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins.

Example of pharmaceutically acceptable carriers that may optionally be used include, but are not limited to aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

In some embodiments the pharmaceutical compositions comprising SNDX-275 are for the treatment of one or more specific disorders. In some embodiments the pharmaceutical compositions are for the treatment of disorders in a mammal, especially a human. In some embodiments the pharmaceutical compositions are for the treatment of cancer such as acute myeloid leukemia, thymus, brain, lung, squamous cell, skin, eye, etc.

### Inhibition of Histone Deacetylase

The invention described herein provides a method of inhibiting histone deacetylase in a cell, comprising contacting a cell in which inhibition of histone deacetylase is desired with an inhibitor of histone deacetylase according to the present invention. Because compounds of the invention inhibit histone deacetylase, they are useful research tools for in vitro study of the role of histone deacetylase in biological processes. In addition, the compounds of the invention selectively inhibit certain isoforms of HDAC.

Measurement of the enzymatic activity of a histone deacetylase can be achieved using known methodologies. For example, Yoshida et al., J. Biol. Chem., 265: 17174-17179 (1990), which is incorporated by reference herein in its entirety, describes the assessment of histone deacetylase enzymatic activity by the detection of acetylated histones in trichostatin A treated cells. Taunton et al., Science, 272: 408-411 (1996), which is incorporated by reference in its entirety, similarly describes methods to measure histone deacetylase enzymatic activity using endogenous and recombinant HDAC-1.

In some embodiments, the histone deacetylase inhibitor interacts with and reduces the activity of all histone deacetylases in the cell. In other embodiments according to this aspect of the invention, the histone deacetylase inhibitor interacts with and reduces the activity of fewer than all histone deacetylases in the cell. In certain other embodiments, the inhibitor interacts with and reduces the activity of one histone deacetylase (e.g., HDAC-1), but does not interact with or reduce the activities of other histone deacetylases (e.g., HDAC-2, HDAC-3, HDAC-4, HDAC-5, HDAC-6, HDAC-7, and HDAC-8). In some embodiments, the histone deacetylase inhibitor of the present invention interacts with, and reduces the enzymatic activity of, a histone deacetylase that is involved in tumorigenesis. In other embodiments, the histone deacetylase inhibitors of the present invention interact with and reduce the enzymatic activity of a fungal histone deacetylase. In some embodiments, SNDX-275 acts as a class I HDAC inhibitor.

In some embodiments, the compounds and methods of the present invention cause an inhibition of cell proliferation of the contacted cells. The phrase “inhibiting cell proliferation” is used to denote an ability of an inhibitor of histone deacetylase to retard the growth of cells contacted with the inhibitor as compared to cells not contacted. An assessment of cell proliferation can be made by counting contacted and non-contacted cells using a Coulter Cell Counter (Coulter, Miami, Fla.) or a hemacytometer. Where the cells are in a solid growth such as, but not limited to, a solid tumor or organ, an assessment of cell proliferation can be made by measuring the growth with calipers and comparing the size of the growth of contacted cells with non-contacted cells. In some embodiments, growth of cells contacted with the inhibitor is retarded by at least 50% as compared to growth of non-contacted cells. In other embodiments, cell proliferation is inhibited by at least 75%. In still other embodiments, cell proliferation is inhibited by 100% (i.e., the contacted cells do not increase in number). Thus, an inhibitor of histone deacetylase according to the invention that inhibits cell proliferation in a contacted cell may induce the contacted cell to undergo growth retardation, to undergo growth arrest, to undergo programmed cell death (i.e., to apoptose), or to undergo necrotic cell death.

### Histone Deacetylase Mediated Disorders

Described herein are compounds, pharmaceutical compositions and methods for treating a patient suffering from a histone deacetylase mediated disorder by administering an effective amount of SNDX-275, or a pharmaceutically acceptable salt, prodrug, solvate, polymorph, tautomer or isomer thereof, alone or in combination with one or more additional active ingredients.

In some embodiments, SNDX-275 is used in the treatment of an inflammatory disease including, but not limited to, asthma, inflammatory bowel diseases such as Crohn's disease and ulcerative colitis, psoriasis, sarcoidois, and rheumatoid arthritis.

In some embodiments, SNDX-275 is used in the treatment of an infection including, but not limited to, malaria, protozoal infections, EBV, HIV, hepatitis B and C, KSHV, toxoplasmosis and coccidiosis.

In some embodiments, SNDX-275 is used in the treatment of an autoimmune disorder including, but not limited to, conditions treatable by immune modulation, rheumatoid arthritis, autoimmune diabetes, lupus, multiple sclerosis, and allergies.

In some embodiments, SNDX-275 is used in the treatment of a neurological disorder including, but not limited to, Huntington's disease, epilepsy, neuropathic pain, depression, and bipolar disorders.

In some embodiments, SNDX-275 is used in the treatment of a proliferative disorder including, but not limited to, psoriasis, restenosis, autoimmune disease, proliferative responses associated with organ transplantation, and atherosclerosis.

In some embodiments, SNDX-275 is used in the treatment of a fibrogenic disorder including, but not limited to, scleroderma, keloid formation, pulmonary fibrosis and liver cirrhosis.

In some embodiments, SNDX-275 is used in the treatment of a cardiac disorder including, but not limited to, cardiovascular conditions, cardiac hypertrophy, idiopathic cardiomyopathies, and heart failure.

In some embodiments, SNDX-275 is used in the treatment of a hyperproliferative disorder including, but not limited to, hematologic and nonhematologic cancers, cancerous and precancerous skin lesions, leukemias, hyperplasias, fibrosis, angiogenesis, psoriasis, atherosclerosis, and smooth muscle proliferation in the blood vessels.

In some embodiments, SNDX-275 is used in the treatment of a metabolic disease including, but not limited to, genetic related metabolic disorders, cystic fibrosis, peroxisome biogenesis disorder, alpha-1 anti-trypsin, adrenoleukodystrophy, and spinal muscular atrophy.

In some embodiments, SNDX-275 is used in the treatment of a malignant disease including, but not limited to, malignant fibrous histiocytoma, malignant mesothelioma, and malignant thymoma.

In some embodiments, SNDX-275 is used in wound healing including, but not limited to, healing of wounds associated with radiation therapy.

In some embodiments, SNDX-275 is used in the treatment of a stroke, ischemia, cancer, tumors, leukemias, neoplasms, or carcinomas, including but not limited to cancer is brain cancer, breast cancer, lung cancer, ovarian cancer, pancreatic cancer, prostate cancer, renal cancer, colorectal cancer, leukemia, myeloid leukemia, glioblastoma, follicular lymphona, pre-B acute leukemia, chronic lymphocytic B-leukemia, mesothelioma or small cell lung cancer. Additional cancers to be treated with the methods and compounds of Formulas I-XI include hematologic and non-hematologic cancers. Hematologic cancer includes multiple myeloma, leukemias, and lymphomas, acute leukemia, acute lymphocytic leukemia (ALL) and acute nonlymphocytic leukemia (ANLL), chronic lymphocytic leukemia (CLL) and chronic myelogenous leukemia (CML). Lymphoma further includes Hodgkin's lymphoma and non-Hodgkin's lymphoma, cutaneous t-cell lymphoma (CTCL) and mantle cell lymphoma (MCL). Non-hematologic cancer includes brain cancer, cancers of the head and neck, lung cancer, breast cancer, cancers of the reproductive system, cancers of the gastro-intestinal system, pancreatic cancer, and cancers of the urinary system, cancer of the upper digestive tract or colorectal cancer, bladder cancer or renal cell carcinoma, and prostate cancer.

In some embodiments, the cancers to treat with the methods and compositions described herein include cancers that are epithelial malignancies (having epithelial origin), and particularly any cancers (tumors) that express EGFR. Non-limiting examples of premalignant or precancerous cancers/tumors having epithelial origin include actinic keratoses, arsenic keratoses, xeroderma pigmentosum, Bowen's disease, leukoplakias, metaplasias, dysplasias and papillomas of mucous membranes, e.g. of the mouth, tongue, pharynx and larynx, precancerous changes of the bronchial mucous membrane such as metaplasias and dysplasias (especially frequent in heavy smokers and people who work with asbestos and/or uranium), dysplasias and leukoplakias of the cervix uteri, vulval dystrophy, precancerous changes of the bladder, e.g. metaplasias and dysplasias, papillomas of the bladder as well as polyps of the intestinal tract. Non-limiting examples of semi-malignant or malignant cancers/tumors of the epithelial origin are breast cancer, skin cancer (e.g., basal cell carcinomas), bladder cancer (e.g., superficial bladder carcinomas), colon cancer, gastro-intestinal (GI) cancer, prostate cancer, uterine cancer, cervical cancer, ovarian cancer, esophageal cancer, stomach cancer, laryngeal cancer and lung cancer.

Additional types of cancers which may be treated using the compositions and methods described herein include: cancers of oral cavity and pharynx, cancers of the respiratory system, cancers of bones and joints, cancers of soft tissue, skin cancers, cancers of the genital system, cancers of the eye and orbit, cancers of the nervous system, cancers of the lymphatic system, and cancers of the endocrine system. These cancers further include cancer of the tongue, mouth, pharynx, or other oral cavity; esophageal cancer, stomach cancer, or cancer of the small intestine; colon cancer or rectal, anal, or anorectal cancer; cancer of the liver, intrahepatic bile duct, gallbladder, pancreas, or other biliary or digestive organs; laryngeal, bronchial, and other cancers of the respiratory organs; heart cancer, melanoma, basal cell carcinoma, squamous cell carcinoma, other non-epithelial skin cancer; uterine or cervical cancer; uterine corpus cancer; ovarian, vulvar, vaginal, or other female genital cancer; prostate, testicular, penile or other male genital cancer; urinary bladder cancer; cancer of the kidney; renal, pelvic, or urethral cancer or other cancer of the genito-urinary organs; thyroid cancer or other endocrine cancer; chronic lymphocytic leukemia; and cutaneous T-cell lymphoma, both granulocytic and monocytic.

Yet other types of cancers which may be treated using the compositions and methods described herein include: adenocarcinoma, angiosarcoma, astrocytoma, acoustic neuroma, anaplastic astrocytoma, basal cell carcinoma, blastoglioma, chondrosarcoma, choriocarcinoma, chordoma, craniopharyngioma, cutaneous melanoma, cystadenocarcinoma, endotheliosarcoma, embryonal carcinoma, ependymoma, Ewing's tumor, epithelial carcinoma, fibrosarcoma, gastric cancer, genitourinary tract cancers, glioblastoma multiforme, hemangioblastoma, hepatocellular carcinoma, hepatoma, Kaposi's sarcoma, large cell carcinoma, leiomyosarcoma, liposarcoma, lymphangiosarcoma, lymphangioendotheliosarcoma, medullary thyroid carcinoma, medulloblastoma, meningioma mesothelioma, myelomas, myxosarcoma neuroblastoma, neurofibrosarcoma, oligodendroglioma, osteogenic sarcoma, epithelial ovarian cancer, papillary carcinoma, papillary adenocarcinomas, parathyroid tumors, pheochromocytoma, pinealoma, plasmacytomas, retinoblastoma, rhabdomyosarcoma, sebaceous gland carcinoma, seminoma, skin cancers, melanoma, small cell lung carcinoma, squamous cell carcinoma, sweat gland carcinoma, synovioma, thyroid cancer, uveal melanoma, and Wilm's tumor.

### Abnormal Cell Growth

Also described herein are compounds, pharmaceutical compositions and methods for inhibiting abnormal cell growth. In some embodiments, the abnormal cell growth occurs in a mammal. Methods for inhibiting abnormal cell growth comprise administering an effective amount of SNDX-275, or a pharmaceutically acceptable salt, prodrug, solvate, polymorph, tautomer or isomer thereof, wherein abnormal cell growth is inhibited. Methods for inhibiting abnormal cell growth in a mammal comprise administering to the mammal an amount of SNDX-275, pharmaceutically acceptable salt, prodrug, solvate, polymorph, tautomer or isomer thereof, wherein the amounts of the compound, pharmaceutically acceptable salt, prodrug, solvate, polymorph, tautomer or isomer thereof, is effective in inhibiting abnormal cell growth in the mammal.

In some embodiments, the methods comprise administering an effective amount of SNDX-275, pharmaceutically acceptable salt, prodrug, solvate, polymorph, tautomer or isomer thereof, in combination with an amount of a chemotherapeutic, wherein the amounts of the compound, or pharmaceutically acceptable salt, prodrug, solvate, polymorph, tautomer or isomer thereof, and of the chemotherapeutic are together effective in inhibiting abnormal cell growth. Many chemotherapeutics are presently known in the art and can be used in combination with the compounds of the invention. In some embodiments, the chemotherapeutic is selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, anti-hormones, angiogenesis inhibitors, and anti-androgens.

Also described are methods for inhibiting abnormal cell growth in a mammal comprising administering to the mammal an amount of SNDX-275, pharmaceutically acceptable salt, prodrug, solvate, polymorph, tautomer or isomer thereof, in combination with radiation therapy, wherein the amounts of the compound, pharmaceutically acceptable salt, prodrug, solvate, polymorph, tautomer or isomer thereof, is in combination with the radiation therapy effective in inhibiting abnormal cell growth or treating the hyperproliferative disorder in the mammal. Techniques for administering radiation therapy are known in the art, and these techniques can be used in the combination therapy described herein. The administration of SNDX-275 in this combination therapy can be determined as described herein.

### Histology of Cancers

In some embodiments, the cancer is of epithelial origin. Non-limiting examples of cancers of epithelial origin are actinic keratoses, arsenic keratoses, xeroderma pigmentosum, Bowen's disease, leukoplakias, metaplasias, dysplasias and papillomas of mucous membranes, e.g. of the mouth, tongue, pharynx and larynx, precancerous changes of the bronchial mucous membrane such as metaplasias and dysplasias (especially frequent in heavy smokers and people who work with asbestos and/or uranium), dysplasias and leukoplakias of the cervix uteri, vulval dystrophy, precancerous changes of the bladder, e.g. metaplasias and dysplasias, papillomas of the bladder as well as polyps of the intestinal tract. Non-limiting examples of semi-malignant or malignant cancers/tumors of the epithelial origin are breast cancer, skin cancer (e.g., basal cell carcinomas), bladder cancer (e.g., superficial bladder carcinomas), colon cancer, gastro-intestinal (GI) cancer, prostate cancer, uterine cancer, cervical cancer, ovarian cancer, esophageal cancer, stomach cancer, laryngeal cancer and lung cancer.

Cancers of epithelial origin can also be identified by similar histology. Common histological markers for epithelial cancers are mucin 16 (CA125), mucin 1, transmembrane (MUC1), mesothelin, WAP four-disulfide core demain 2 (HE4), kallikrein 6, kallikrein 10, matrix metallopreinase 2, prostasin, osteopontin, tetranectin, and inhibin. Additional histological markers include prostate-specific antigen (PSA), MUC6, IEN, and aneuploidy. Additional examples of histological markers for epithelial cancers include E-cadherin, EZH2, Nectin-4, Her-2, p53, Ki-67, ErbB3, ZEB1 and/or SIP1 expression.

In some embodiments, the cancer is a hematological cancer. Non-limiting examples of hematological cancers include lymphoma (including, but not limited to, Hodgkin's lymphoma, diffuse large b-cell lymphoma (DLBCL) also know as immunoblastic lymphoma, aggressive lymphomas also known as intermediate and high grade lymphomas, indolent lymphomas also known as low grade lymphomas, mantle cell lymphoma, follicular lymphoma), leukemia, acute promyelocytic leukemia, acute myeloideleukaemia, chronic myeloide leukaemia, chronic lymphatic leukaemia, Hodgkin's disease, multiple myeloma, myelodysplasia, myeloproliferative disease, and refractory anemia.

Hematological cancers can also be identified by similar histology. Common histological markers for hematological cancers are tumor-antigens, M34, antibodies, cancer antigens, CA15-3, carcinoembryonic antigen, CA125, cytokeratins, hMAM, MAGE, pancytokeratins, and HLA Class I or Class II antigens such as HLA-DR and HLA-D, MB, MT, MTe, Te, and SB. Additional examples of histological markers for B-cell malignancies include CD5, CD6, CD10, CD19, CD20, CD21, CD22, CD23, CD24, CD25, CD26, CD28, CD30, CD32, CD35, CD37, CD38, CD39, CD40, CD43, CD45RO, CD45RA, CD45RB, CD49B, CD49C, CD49D, CD50, CD52, CD57, CD62L, CD69, CD70, CD72, CD73, CD74, CD75, CD77, CD79 [symbol] , [symbol] , CD80, CD83, CDW84, CD86, CD89, CD97, CD98, CD119, CDW121B, CD122, CD124, CD125, CD126, CD127, CD130, CD132, CD135, CDW137, CD171, CD179A, CD179B, CD180, CD183, CDW197, CD200, CDW210, CD213A1 and CD213A2. Examples of histological markers for T-cell malignancies include CD4, CD8, CD5, CD2, CD25, CD26, CD28, CD27, CD30, CD37, CD38, CD45RO, CD45RA, CD45RB, CD49A, CD49E, CD49F, CD50, CD52, CD56, CD57, CD62L, CD69, CD70, CD73, CD89, CD90, CD94, CD96, CD97, CD98, CD101, CD107A, CD107B, CD109, CD121A, CD122, CD124, CDW128, CD132, CD134, CDW137, CD148, CD152, CD153, CD154, CD160, CD161, CD165, CD166, CD171, CD178, CDW197, CDW210, CD212, CDW217, CD223, CD226, CD231, CD245 and CD247.

In some embodiments, the cancer is a neuroendocrine cancer. Non-limiting examples of neuroendocrine cancers include lung and pancreatic cancers as well as neuroendocrine tumors of the digestive system. More specifically, these types of cancer may be called gastrinoma, insulinoma, glucagonoma, vasoactive intestinal peptideoma (VIPoma), PPoma, somatostatinoma, CRHoma, calcitoninoma, GHRHoma, ACTHoma, and GRFoma. Additional examples of neuroendocrine cancers include medullary carcinoma of the thyroid, Merkel cell cancer, small-cell lung cancer (SCLC), large-cell neuroendocrine carcinoma of the lung, neuroendocrine carcinoma of the cervix, Multiple Endocrine Neoplasia type 1 (MEN-1 or MEN1), Multiple Endocrine Neoplasia type 2 (MEN-2 or MEN2), neurofibromatosis type 1, tuberous sclerosis, von Hippel-Lindau (VHL) disease, neuroblastoma, pheochromocytoma (phaeochromocytoma), paraganglioma, neuroendocrine tumor of the anterior pituitary, and Carney's complex.

Neuroendocrine cancers can also be identified by similar histology. Common histological markers for neuroendocrine cancers are hormone markers, chromogranin A (CgA), urine 5-hydroxy indole acetic acid (5-HIAA) (grade C), neuron-specific enolase (NSE, gamma-gamma dimer), synaptophysin (P38), N-terminally truncated variant of heat shock protein 70 (Hsp 70), CDX-2, neuroendocrine secretory protein-55, and blood serotonin.

Other histological markers are known in the art provide the ability to potentially identify and distinguish cancer cells from normal cells or within different types of cancers or malignancies.

### Modes of Administration

SNDX-275 may be prepared as a free base or a pharmaceutically acceptable salt, solvate, polymorph, ester, tautomer or prodrug thereof. Also described, are pharmaceutical compositions comprising SNDX-275 or a pharmaceutically acceptable salt, solvate, polymorph, ester, tautomer or prodrug thereof. The compounds and compositions described herein may be administered either alone or in combination with pharmaceutically acceptable carriers, excipients or diluents, in a pharmaceutical composition, according to standard pharmaceutical practice. In some embodiments, SNDX-275 is formulated as a solid dosage form, such as a tablet, capsule, caplet, powder, etc. In some embodiments, SNDX-275 is formulated as a tablet, wherein the tablet contains from about 0.1 to about 12 mg, e.g. about 1, 2, 3, 4, 5, 6, 7, 8, 9, 10, 11 or 12 mg. In some embodiments, SNDX-275 is formulated as a tablet containing 2, 3, 4, 5, 7 or 10 mg of SNDX-275.

Administration of the compounds and compositions described herein can be effected by any method that enables delivery of the compounds to the site of action. These methods include oral routes, intraduodenal routes, parenteral injection (including intravenous, subcutaneous, intraperitoneal, intramuscular, intravascular or infusion), topical, intrapulmonary, rectal administration, by implant, by a vascular stent impregnated with the compound, and other suitable methods commonly known in the art. For example, compounds described herein can be administered locally to the area in need of treatment. This may be achieved by, for example, but not limited to, local infusion during surgery, topical application, e.g., cream, ointment, injection, catheter, or implant, said implant made, e.g., out of a porous, non-porous, or gelatinous material, including membranes, such as sialastic membranes, or fibers. The administration can also be by direct injection at the site (or former site) of a tumor or neoplastic or pre-neoplastic tissue. Those of ordinary skill in the art are familiar with formulation and administration techniques that can be employed with the compounds and methods of the invention, e.g., as discussed in Goodman and Gilman, The Pharmacological Basis of Therapeutics, (current edition); Pergamon; and Remington's, Pharmaceutical Sciences (current edition), Mack Publishing Co., Easton, Pa.

The formulations include those suitable for oral, parenteral (including subcutaneous, intradermal, intramuscular, intravenous, intraarticular, intramedullary, intracardiac, intrathecal, intraspinal, intracapsular, subcapsular, intraorbital, intratracheal, subcuticular, intraarticular, subarachnoid, and intrastemal), intraperitoneal, transmucosal, transdermal, rectal and topical (including dermal, buccal, sublingual, intranasal, intraocular, and vaginal) administration although the most suitable route may depend upon for example the condition and disorder of the recipient. The formulations may conveniently be presented in unit dosage form and may be prepared by any of the methods well known in the art of pharmacy. All methods include the step of bringing into association SNDX-275 or a pharmaceutically acceptable salt, ester, prodrug or solvate thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

Formulations suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

Pharmaceutical preparations which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders (e.g., povidone, gelatin, hydroxypropylmethyl cellulose), inert diluents, preservative, disintegrant (e.g., sodium starch glycolate, cross-linked povidone, cross-linked sodium carboxymethyl cellulose) or lubricating, surface active or dispersing agents. Molded tablets may be made by molding in a suitable machine a mixture of the powdered compound moistened with an inert liquid diluent. The tablets may optionally be coated or scored and may be formulated so as to provide slow or controlled release of the active ingredient therein. Tablets may optionally be provided with an enteric coating, to provide release in parts of the gut other than the stomach. All formulations for oral administration should be in dosages suitable for such administration. In some embodiments, the formulations may be provided in a gastric retentive system. In other embodiments, the formulations are provided in a modified release system. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or Dragee coatings for identification or to characterize different combinations of active compound doses.

Pharmaceutical preparations may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Formulations for parenteral administration include aqueous and non-aqueous (oily) sterile injection solutions of the active compounds which may contain antioxidants, buffers, biocide, bacteriostats and solutes which render the formulation isotonic with the blood of the intended recipient; and aqueous and non-aqueous sterile suspensions which may include suspending agents and thickening agents. Examples of suitable isotonic vehicles for use in such formulations include Sodium Chloride Injection, Ringer's Solution, or Lactated Ringer's Injection. Suitable lipophilic solvents or vehicles include fatty oils such as sesame oil, or synthetic fatty acid esters, such as ethyl oleate or triglycerides, or liposomes or other microparticulate systems may be used to target the compound to blood components or one or more organs. The concentration of the active ingredient in the solution may vary widely. Typically, the concentration of the active ingredient in the solution is from about 1 ng/ml to about 10 μg/ml, for example from about 10 ng/ml to about 1 μg/ml. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions

Pharmaceutical preparations may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

Pharmaceutical preparations may also be formulated in rectal compositions such as suppositories or retention enemas, e.g., containing conventional suppository bases such as cocoa butter, polyethylene glycol, or other glycerides.

Pharmaceutical preparations may be administered topically, that is by non-systemic administration. This includes the application of SNDX-275 externally to the epidermis or the buccal cavity and the instillation of such The compound into the ear, eye and nose, such that the compound does not significantly enter the blood stream. In contrast, systemic administration refers to oral, intravenous, intraperitoneal and intramuscular administration.

Pharmaceutical preparations suitable for topical administration include liquid or semi-liquid preparations suitable for penetration through the skin to the site of inflammation such as gels, liniments, lotions, creams, ointments or pastes, suspensions, powders, solutions, spray, aerosol, oil, and drops suitable for administration to the eye, ear or nose. Alternatively, a formulation may comprise a patch or a dressing such as a bandage or adhesive plaster impregnated with active ingredients and optionally one or more excipients or diluents. The amount of active ingredient present in the topical formulation may vary widely. The active ingredient may comprise, for topical administration, from 0.001% to 10% w/w, for instance from 1% to 2% by weight of the formulation. It may however comprise as much as 10% w/w but preferably will comprise less than 5% w/w, more preferably from 0.1% to 1% w/w of the formulation.

Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Formulations suitable for topical administration to the eye also include eye drops wherein the active ingredient is dissolved or suspended in a suitable carrier, especially an aqueous solvent for the active ingredient.

Pharmaceutical preparations for administration by inhalation are conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, pharmaceutical preparations may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

It should be understood that in addition to the ingredients particularly mentioned above, the compounds and compositions described herein may include other agents conventional in the art having regard to the type of formulation in question, for example those suitable for oral administration may include flavoring agents.

### Formulations

The compounds or compositions described herein can be delivered in a vesicle, e.g., a liposome (see, for example, Langer, Science 1990, 249,1527-1533; Treat et al., Liposomes in the Therapy of Infectious Disease and Cancer, Lopez-Bernstein and Fidler, Ed., Liss, N.Y., pp. 353-365, 1989). The compounds and pharmaceutical compositions described herein can also be delivered in a controlled release system. In some embodiments, a pump may be used (see, Sefton, 1987, CRC Crit. Ref Biomed. Eng. 14:201; Buchwald et al. Surgery, 1980 88, 507; Saudek et al. N. Engl. J. Med. 1989, 321, 574. Additionally, a controlled release system can be placed in proximity of the therapeutic target. (See, Goodson, Medical Applications of Controlled Release, 1984, Vol. 2, pp. 115-138). The pharmaceutical compositions described herein can also contain the active ingredient in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions, and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, such as microcrystalline cellulose, sodium crosscarmellose, corn starch, or alginic acid; binding agents, for example starch, gelatin, polyvinyl-pyrrolidone or acacia, and lubricating agents, for example, magnesium stearate, stearic acid or talc. The tablets may be un-coated or coated by known techniques to mask the taste of the drug or delay disintegration and absorption in the gastrointestinal tract and thereby provide a sustained action over a longer period. For example, a water soluble taste masking material such as hydroxypropylmethyl-cellulose or hydroxypropylcellulose, or a time delay material such as ethyl cellulose, or cellulose acetate butyrate may be employed as appropriate. Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water soluble carrier such as polyethyleneglycol or an oil medium, for example peanut oil, liquid paraffin, or olive oil.

Aqueous suspensions contain the active material in admixture with excipients suitable for the manufacture of aqueous suspensions. Such excipients are suspending agents, for example sodium carboxymethylcellulose, methylcellulose, hydroxypropylmethyl-cellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as butylated hydroxyanisol or alpha-tocopherol.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present. These compositions may be preserved by the addition of an anti-oxidant such as ascorbic acid.

Pharmaceutical compositions may also be in the form of an oil-in-water emulsions. The oily phase may be a vegetable oil, for example olive oil or arachis oil, or a mineral oil, for example liquid paraffin or mixtures of these. Suitable emulsifying agents may be naturally-occurring phosphatides, for example soy bean lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening agents, flavoring agents, preservatives and antioxidants.

Syrups and elixirs may be formulated with sweetening agents, for example glycerol, propylene glycol, sorbitol or sucrose. Such formulations may also contain a demulcent, a preservative, flavoring and coloring agents and antioxidant.

Pharmaceutical compositions may be in the form of a sterile injectable aqueous solution. Among the acceptable vehicles and solvents that may be employed are water, Ringer's solution and isotonic sodium chloride solution. The sterile injectable preparation may also be a sterile injectable oil-in-water microemulsion where the active ingredient is dissolved in the oily phase. For example, the active ingredient may be first dissolved in a mixture of soybean oil and lecithin. The oil solution then introduced into a water and glycerol mixture and processed to form a microemulsion. The injectable solutions or microemulsions may be introduced into a patient's blood-stream by local bolus injection. Alternatively, it may be advantageous to administer the solution or microemulsion in such a way as to maintain a constant circulating concentration of the instant compound. In order to maintain such a constant concentration, a continuous intravenous delivery device may be utilized. An example of such a device is the Deltec CADD-PLUS™ model 5400 intravenous pump. The pharmaceutical compositions may be in the form of a sterile injectable aqueous or oleaginous suspension for intramuscular and subcutaneous administration. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butanediol. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Pharmaceutical compositions may also be administered in the form of suppositories for rectal administration of the drug. These compositions can be prepared by mixing the inhibitors with a suitable non-irritating excipient which is solid at ordinary temperatures but liquid at the rectal temperature and will therefore melt in the rectum to release the drug. Such materials include cocoa butter, glycerinated gelatin, hydrogenated vegetable oils, mixtures of polyethylene glycols of various molecular weights and fatty acid esters of polyethylene glycol.

For topical use, creams, ointments, jellies, solutions or suspensions, etc., containing the compound or composition of the invention can be used. As used herein, topical application can include mouth washes and gargles.

Pharmaceutical compositions may be administered in intranasal form via topical use of suitable intranasal vehicles and delivery devices, or via transdermal routes, using those forms of transdermal skin patches well known to those of ordinary skill in the art. To be administered in the form of a transdermal delivery system, the dosage administration will, of course, be continuous rather than intermittent throughout the dosage regimen.

### Doses

In some embodiments, the suitable dose of SNDX-275 is administered to a patient in a fed state. For example, the SNDX-275 may be administered up to 6 hours after a meal, or within 4 hours of a meal, or within 2 hours of a meal or within 1 hour of a meal. In other embodiments, the suitable dose of SNDX-275 is administered to a patient in a fasted state. In some embodiments, the suitable dose of a second therapeutic is administered in the fed state. In other embodiments, the suitable dose of the second therapeutic is administered in the fasted state. In various embodiments, SNDX-275 is administered to a patient in the fed state and a second therapeutic is administered to the patient in a fasted state.

In some embodiments, suitable dosages of SNDX-275 are total weekly dosages of between about 0.25 to about 10 mg/m². They can be administered in various cycles: once weekly at a dose of about 2 to 10 mg; twice weekly at a dose of about 0.5 to about 2 mg; once every other week (biweekly) at a dose of about 2 to 12 mg; three times monthly at a dose of about 2 to 10 mg; four times per six weeks (e.g. four weeks on and two weeks off) at 2 to 10 mg, two times monthly (e.g. 2 weeks on and 2 weeks off) at a dose of 2 to 10 mg.

In some embodiments, so called “fixed” dosing of SNDX-275 may be employed. A fixed dose is a particular mass of SNDX-275: that is neither the mass nor the surface area of the patient are taken into account when determining the dose. Suitable fixed doses contemplated herein are about 0.25, 0.5, 0.75, 1, 2, 3, 4, 5, 6, 7, 8, 9, 10, 11 or 12 mg of SNDX-275 per dose. Particular fixed doses contemplated herein are 3, 5, 7 and 10 mg of SNDX-275 per dose. Such doses may be administered on one of dosing schedules described herein. In some embodiments, a dose of about 0.25, 0.5, 0.75, 1, 2, 3, 4, 5, 6, 7, 8, 9, 10, 11 or 12 mg of SNDX-275 per dose is administered on a twice-weekly, weekly (once per week) or biweekly (once every other week) dosing schedule, optionally with a rest period built in after a certain number of dosing cycles. In some embodiments, the dosing schedule is weekly and SNDX-275 is administered at a dose of about 1-12 mg (e.g. about 2, 3, 4, 5, 6, 7, 8, 9 or 10 mg) once a week for two weeks, followed by a rest period (i.e. no chemotherapy) of one, two or three weeks. In some embodiments, the dosing schedule is weekly and SNDX-275 is administered at a dose of about 1-12 mg (e.g. about 2, 3, 4, 5, 6, 7, 8, 9 or 10 mg) once a week for three weeks, followed by a rest period of one, two or three weeks. In some embodiments, the dosing schedule is weekly and SNDX-275 is administered at a dose of about 1-12 mg (e.g. about 2, 3, 4, 5, 6, 7, 8, 9 or 10 mg) once a week for four weeks, followed by a rest period of one, two or three weeks. In some embodiments, the dosing schedule is twice weekly (2×weekly) and SNDX-275 is administered at a dose of about 0.25 to about 8 mg (e.g. about 0.25, 0.5, 0.75, 1, 2, 3, 4, 5 or 6 mg) twice a week for two weeks, followed by a rest period (i.e. no chemotherapy) of one, two or three weeks. In some embodiments, the dosing schedule is 2×weekly and SNDX-275 is administered at a dose of about 0.25 to about 8 mg (e.g. about 0.25, 0.5, 0.75, 1, 2, 3, 4, 5 or 6 mg) twice a week for three weeks, followed by a rest period of one, two or three weeks. In some embodiments, the dosing schedule is 2×weekly and SNDX-275 is administered at a dose of about 0.25 to about 8 mg (e.g. about 0.25, 0.5, 0.75, 1, 2, 3, 4, 5 or 6 mg) twice a week for four weeks, followed by a rest period of one, two or three weeks. In some embodiments, the dosing schedule is every other week (biweekly) and SNDX-275 is administered at a dose of about 2-12 mg (e.g. about 2, 3, 4, 5, 6, 7, 8, 9 or 10 mg) once a biweek (once every other week).

In some embodiments, the total dosage range is about 1 mg to about 12 mg/m² per biweek. In some embodiments, the total dosage range is about 1 mg to about 12 mg/m² per week. In some embodiments, a total dosage will range from about 2 to about 24 mg/m² per month.

SNDX-275 can also be administered in combination with at least one second chemotherapeutic compound (e.g. pharmaceuticals, small-molecule compounds, antibodies and fragments thereof, immune system modulating proteins, antibiotics, or other biologic therapy), radiotherapy, or surgery. Such co-administration is believed to increase efficacy, provide synergistic effect, and/or provide increased therapeutic value to each agent, compound, or additional treatment (e.g. radiotherapy or surgery).

In some embodiments, the compound described herein is administered with a second chemotherapeutic compound. The co-administered compounds can be administered in a variety of cycles: the compound can be administered continuously, daily, every other day, every third day, once a week, twice a week, three times a week, bi-weekly, or monthly, while the second chemotherapeutic agent is administered continuously, daily, one day a week, two days a week, three days a week, four days a week, five days a week, six days a week, bi-weekly, or monthly. The compound and the second chemotherapeutic compound or cancer can be administered in, but are not limited to, any combination of the aforementioned cycles. In one non-limiting example, the compound is administered three times a week for the first two weeks followed by no administration for four weeks, and the second chemotherapeutic compound is administered continuously over the same six week period. In yet another non-limiting example, the compound is administered once a week for six weeks, and the second chemotherapeutic compound is administered every other day over the same six week period. In yet another non-limiting example, the compound is administered the first two days of a week, and the second chemotherapeutic compound is administered continuously for all seven days of the same week. The compound can be administered before, with or after the second chemotherapeutic compound is administered.

In addition to the administration of the compounds in cycles, the cycles themselves may consist of varying schedules. In some embodiments, a cycle is administered weekly. In other embodiments, a cycle is administered with one, two, three, four, five, six, or seven days off before repeating the cycle. In additional embodiments, a cycle is administered for one week with one, two, three, four, six, or eight weeks off before repeating the cycle. In further embodiments, a cycle is administered for two weeks with one, two, three, four, six, or eight weeks off before repeating the cycle. In still further embodiments, the cycle is administered for three, four, five, or six weeks, with one, two, three, four, six, or eight weeks off before repeating the cycle.

When a compound is administered with an additional treatment such as radiotherapy, the radiotherapy can be administered at 1 day, 2 days, 3 days, 4 days, 5 days, 6 days, 7 days, 14 days, 21 days, or 28 days after administration of at least one cycle of a compound. Alternatively, the radiotherapy can be administered at 1 day, 2 days, 3 days, 4 days, 5 days, 6 days, 7 days, 14 days, 21 days, or 28 days before administration of at least one cycle of a compound. In additional embodiments, the radiotherapy can be administered in any variation of timing with any variation of the aforementioned cycles for a compound. Additional schedules for co-administration of radiotherapy with cycles of a compound will be known in the art, can be further determined by appropriate testing, clinical trials, or can be determined by qualified medical professionals.

When a compound is administered with an additional treatment such as surgery, the compound is administered 1, 2, 3, 4, 5, 6, 7, 14, 21, or 28 days prior to surgery. In additional embodiments, at least one cycle of the compound is administered 1, 2, 3, 4, 5, 6, 7, 14, 21, or 28 days after surgery. Additional variations of administering compound cycles in anticipation of surgery, or after the occurrence of surgery, will be known in the art, can be further determined by appropriate testing and/or clinical trials, or can be determined by assessment of qualified medical professionals.

In addition to the aforementioned examples and embodiments of dosages, cycles, and schedules of cycles, numerous permutations of the aforementioned dosages, cycles, and schedules of cycles for the co-administration of a compound with a second chemotherapeutic compound, radiotherapy, or surgery are contemplated herein and can be administered according to the patient, type of cancer, and/or appropriate treatment schedule as determined by qualified medical professionals.

### Dosage Forms

The pharmaceutical composition may, for example, be in a form suitable for oral administration as a tablet, capsule, cachet, pill, lozenge, powder or granule, sustained release formulations, solution, liquid, suspension, for parenteral injection as a sterile solution, suspension or emulsion, for topical administration as an ointment, cream, lotions, sprays, foams, gel or paste, or for rectal or vaginal administration as a suppository or pessary. The pharmaceutical composition may be in unit dosage forms suitable for single administration of precise dosages. The pharmaceutical composition will include a conventional pharmaceutical carrier or excipient and the compound according to the invention as an active ingredient. In addition, it may include other medicinal or pharmaceutical agents, carriers, adjuvants, etc.

Exemplary parenteral administration forms include solutions or suspensions of active compounds in sterile aqueous solutions, for example, aqueous propylene glycol or dextrose solutions. Such dosage forms can be suitably buffered, if desired.

Suitable pharmaceutical carriers include inert diluents or fillers, water and various organic solvents. The pharmaceutical compositions may, if desired, contain additional ingredients such as flavorings, binders, excipients and the like. Thus for oral administration, tablets containing various excipients, such as citric acid may be employed together with various disintegrants such as starch or other cellulosic material, alginic acid and certain complex silicates and with binding agents such as sucrose, gelatin and acacia. Additionally, lubricating agents such as magnesium stearate, sodium lauryl sulfate and talc are often useful for tableting purposes. Other reagents such as an inhibitor, surfactant or solubilizer, plasticizer, stabilizer, viscosity increasing agent, or film forming agent may also be added. Solid compositions of a similar type may also be employed in soft and hard filled gelatin capsules. Preferred materials, therefore, include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with diluents such as water, ethanol, propylene glycol, glycerin, or combinations thereof.

Methods of preparing various pharmaceutical compositions with a specific amount of active compound are known, or will be apparent, to those skilled in this art. For examples, see Remington's Pharmaceutical Sciences, Mack Publishing Company, Ester, Pa., 18th Edition (1990).

### Combination Therapies

SNDX-275 or a pharmaceutically acceptable salt, prodrug, solvate, polymorph, tautomer or isomer thereof may be administered as a sole therapy. SNDX-275 and their pharmaceutically acceptable salts, prodrug, solvates, polymorphs, tautomers or isomers may also be administered in combination with another cancer therapy or therapies. As described above, these additional cancer therapies can be, for example, surgery, radiation therapy, administration of chemotherapeutic agents and combinations of any two or all of these methods. Combination treatments may occur sequentially or concurrently and the combination therapies may be neoadjuvant therapies or adjuvant therapies.

In some embodiments, SNDX-275 can be administered with an additional therapeutic agent. In these embodiments, the compound described herein can be in a fixed combination with the additional therapeutic agent or a non-fixed combination with the additional therapeutic agent.

In applications with administration of a therapeutic agent for treatment of side effects with the combination treatments as described, the therapeutic agent for treatment of side effects may be administered concurrently (e.g., simultaneously, essentially simultaneously or within the same treatment protocol) or sequentially, depending upon the nature and onset of the side effect, the condition of the patient, and the actual choice of chemotherapeutic agent and/or radiation to be administered in conjunction (i.e., within a single treatment protocol) with the compound/composition. For a non-limiting example, an anti-nausea drug may be prophylactically administered prior to combination treatment with the compound and radiation therapy. For another non-limiting example, an agent for rescuing immuno -suppressive side effects is administered to the patient subsequent to the combination treatment of compound and another chemotherapeutic agent. The routes of administration for the therapeutic agent for side effects can also differ than the administration of the combination treatment. The determination of the mode of administration for treatment of side effects and the advisability of administration, where possible, in the same pharmaceutical composition, is within the knowledge of the skilled clinician with the teachings described herein. The initial administration can be made according to established protocols known in the art, and then, based upon the observed effects, the dosage, modes of administration and times of administration can be modified by the skilled clinician. The particular choice of therapeutic agent for treatment of side effects will depend upon the diagnosis of the attending physicians and their judgment of the condition of the patient and the appropriate treatment protocol.

In some embodiments, therapeutic agents specific for treating side effects may by administered before the administration of the combination treatment described. In other embodiments, therapeutic agents specific for treating side effects may by administered simultaneously with the administration of the combination treatment described. In another embodiments, therapeutic agents specific for treating side effects may by administered after the administration of the combination treatment described.

In some embodiments, therapeutic agents specific for treating side effects may include, but are not limited to, anti-emetic agents, immuno-restorative agents, antibiotic agents, anemia treatment agents, and analgesic agents for treatment of pain and inflammation.

Anti-emetic agents are a group of drugs effective for treatment of nausea and emesis (vomiting). Cancer therapies frequently cause urges to vomit and/or nausea. Many anti-emetic drugs target the 5-HT₃ seratonin receptor which is involved in transmitting signals for emesis sensations. These 5-HT₃ antagonists include, but are not limited to, dolasetron (Anzemet®), granisetron (Kytril®), ondansetron (Zofran®), palonosetron and tropisetron. Other anti-emetic agents include, but are not limited to, the dopamine receptor antagonists such as chlorpromazine, domperidone, droperidol, haloperidol, metaclopramide, promethazine, and prochlorperazine; antihistamines such as cyclizine, diphenhydramine, dimenhydrinate, meclizine, promethazine, and hydroxyzine; lorazepram, scopolamine, dexamethasone, emetrol®, propofol, and trimethobenzamide. Administration of these anti-emetic agents in addition to the above described combination treatment will manage the potential nausea and emesis side effects caused by the combination treatment.

Immuno-restorative agents are a group of drugs that counter the immuno-suppressive effects of many cancer therapies. The therapies often cause myelosuppression, a substantial decrease in the production of leukocytes (white blood cells). The decreases subject the patient to a higher risk of infections. Neutropenia is a condition where the concentration of neutrophils, the major leukocyte, is severely depressed Immuno-restorative agents are synthetic analogs of the hormone, granulocyte colony stimulating factor (G-CSF), and act by stimulating neutrophil production in the bone marrow. These include, but are not limited to, filgrastim (Neupogen®), PEG-filgrastim (Neulasta®) and lenograstim. Administration of these immuno-restorative agents in addition to the above described combination treatment will manage the potential myelosupression effects caused by the combination treatment.

Antibiotic agents are a group of drugs that have anti-bacterial, anti-fungal, and anti-parasite properties. Antibiotics inhibit growth or causes death of the infectious microorganisms by various mechanisms such as inhibiting cell wall production, preventing DNA replication, or deterring cell proliferation. Potentially lethal infections occur from the myelosupression side effects due to cancer therapies. The infections can lead to sepsis where fever, widespread inflammation, and organ dysfunction arise. Antibiotics manage and abolish infection and sepsis and include, but are not limited to, amikacin, gentamicin, kanamycin, neomycin, netilmicin, streptomycin, tobramycin, loracarbef, ertapenem, cilastatin, meropenem, cefadroxil, cefazolin, cephalexin, cefaclor, cefamandole, cefoxitin, cefprozil, cefuroxime, cefixime, cefdinir, cefditoren, cefoperazone, cefotaxime, cefpodoxime, ceftazidime, ceftibuten, ceftizoxime, ceftriaxone, cefepime, teicoplanin, vancomycin, azithromycin, clarithromycin, dirithromycin, erthromycin, roxithromycin, troleandomycin, aztreonam, amoxicillin, ampicillin, azlocillin, carbenicillin, cloxacillin, dicloxacillin, flucloxacillin, mezlocillin, nafcillin, penicillin, piperacillin, ticarcillin, bacitracin, colistin, polymyxin B, ciprofloxacin, enoxacin, gatifloxacin, levofloxacin, lomefloxacin, moxifloxacin, norfloxacin, ofloxacin, trovafloxacin, benzolamide, bumetanide, chlorthalidone, clopamide, dichlorphenamide, ethoxzolamide, indapamide, mafenide, mefruside, metolazone, probenecid, sulfanilamides, sulfamethoxazole, sulfasalazine, sumatriptan, xipamide, democlocycline, doxycycline, minocycline, oxytetracycline, tetracycline, chloramphenical, clindamycin, ethambutol, fosfomycin, fusidic acid, furazolidone, isoniazid, linezolid, metronidazole, mupirocin, nitrofurantoin, platesimycin, pyrazinamide, dalfopristin, rifampin, spectinomycin, and telithromycin. Administration of these antibiotic agents in addition to the above described combination treatment will manage the potential infection and sepsis side effects caused by the combination treatment.

Anemia treatment agents are compounds directed toward treatment of low red blood cell and platelet production. In addition to myelosuppression, many cancer therapies also cause anemias, deficiencies in concentrations and production of red blood cells and related factors. Anemia treatment agents are recombinant analogs of the glycoprotein, erythropoeitin, and function to stimulate erythropoesis, the formation of red blood cells. Anemia treatment agents include, but are not limited to, recombinant erythropoietin (EPOGEN®, Dynopro®) and Darbepoetin alfa (Aranesp®). Administration of these anemia treatment agents in addition to the above described combination treatment will manage the potential anemia side effects caused by the combination treatment.

Pain and inflammation side effects arising from the described herein combination treatment may be treated with compounds selected from the group comprising: corticosteroids, non-steroidal anti-inflammatories, muscle relaxants and combinations thereof with other agents, anesthetics and combinations thereof with other agents, expectorants and combinations thereof with other agents, antidepressants, anticonvulsants and combinations thereof; antihypertensives, opioids, topical cannabinoids, and other agents, such as capsaicin.

For the treatment of pain and inflammation side effects, compounds according to the present invention may be administered with an agent selected from the group comprising: betamethasone dipropionate (augmented and nonaugmented), betamethasone valerate, clobetasol propionate, prednisone, methyl prednisolone, diflorasone diacetate, halobetasol propionate, amcinonide, dexamethasone, dexosimethasone, fluocinolone acetononide, fluocinonide, halocinonide, clocortalone pivalate, dexosimetasone, flurandrenalide, salicylates, ibuprofen, ketoprofen, etodolac, diclofenac, meclofenamate sodium, naproxen, piroxicam, celecoxib, cyclobenzaprine, baclofen, cyclobenzaprine/lidocaine, baclofen/cyclobenzaprine, cyclobenzaprine/lidocaine/ketoprofen, lidocaine, lidocaine/deoxy-D-glucose, prilocaine, EMLA Cream (Eutectic Mixture of Local Anesthetics (lidocaine 2.5% and prilocaine 2.5%), guaifenesin, guaifenesin/ketoprofen/cyclobenzaprine, amitryptiline, doxepin, desipramine, imipramine, amoxapine, clomipramine, nortriptyline, protriptyline, duloxetine, mirtazepine, nisoxetine, maprotiline, reboxetine, fluoxetine, fluvoxamine, carbamazepine, felbamate, lamotrigine, topiramate, tiagabine, oxcarbazepine, carbamezipine, zonisamide, mexiletine, gabapentin/clonidine, gabapentin/carbamazepine, carbamazepine/cyclobenzaprine, antihypertensives including clonidine, codeine, loperamide, tramadol, morphine, fentanyl, oxycodone, hydrocodone, levorphanol, butorphanol, menthol, oil of wintergreen, camphor, eucalyptus oil, turpentine oil; CB1/CB2 ligands, acetaminophen, infliximab) nitric oxide synthase inhibitors, particularly inhibitors of inducible nitric oxide synthase; and other agents, such as capsaicin. Administration of these pain and inflammation analgesic agents in addition to the above described combination treatment will manage the potential pain and inflammation side effects caused by the combination treatment.

Kits for Co-Administration of SNDX-275 with Other Pharmaceutically Active Agents

As discussed above, in some embodiments, SNDX-275 may be administered as a monotherapy or may be combined with one or more active pharmaceutical ingredients the treatment cancer. In particular, SNDX-275 may be co-administered with a compound that works synergistically with SNDX-275 and/or treats one of the sequelae of cancer or of cancer treatment, such as nausea, emesis, alopecia, fatigue, anorexia, anhedonia, depression, immunosuppression, infection, etc.

In some embodiments, the invention provides a kit including SNDX-275 in a dosage form, especially a dosage form for oral administration. Thus, in some embodiments of the invention, the kit includes one or more doses of SNDX-275 in tablets for oral administration. In other embodiments, however, the doses of SNDX-275 may be present in a variety of dosage forms, such as capsules, caplets, gel caps, powders for suspension, etc.

In some embodiments, a kit according to the invention includes at least two dosage forms, one comprising a first active pharmaceutical ingredient (SNDX-275) and the other comprising at least a second active pharmaceutical ingredient, other than the first active pharmaceutical ingredient. In some embodiments, the kit includes sufficient doses for a period of time. In particular embodiments, the kit includes a sufficient dose of each active pharmaceutical ingredient for a day, a week, 14 days, 28 days, 30 days, 90 days, 180 days, a year, etc. It is considered that the most convenient periods of time for which such kits are designed would be from 1 to 13 weeks, especially 1 week, 2 weeks, 1 month, 3 months, etc. In some specific embodiments, the each dose is physically separated into a compartment, in which each dose is segregated from the others.

In some embodiments, the kit according to the invention includes at least two dosage forms, one comprising SNDX-275 and the other comprising at least one active pharmaceutical ingredient other than SNDX-275. In some embodiments, the kit includes sufficient doses for a period of time. In particular embodiments, the kit includes a sufficient dose of each active pharmaceutical ingredient for a day, a week, 14 days, 28 days, 30 days, 90 days, 180 days, a year, etc. In some specific embodiments, the each dose is physically separated into a compartment, in which each dose is segregated from the others.

In particular embodiments, the kit may advantageously be a blister pack. Blister packs are known in the art, and generally include a clear side having compartments (blisters or bubbles), which separately hold the various doses, and a backing, such as a paper, foil, paper-foil or other backing, which is easily removed so that each dose may be separately extracted from the blister pack without disturbing the other doses. In some embodiments, the kit may be a blister pack in which each dose of SNDX-275 and at least a second active pharmaceutical ingredient are segregated from the other doses in separate blisters or bubbles. In some such embodiments, the blister pack may have perforations, which allow each daily dose to be separated from the others by tearing it away from the rest of the blister pack. The separate dosage forms may be contained within separate blisters. Segregation of the two active pharmaceutical ingredients into separate blisters can be advantageous in that it prevents separate dosage forms (e.g. tablet and capsule) from contacting and damaging one another during shipping and handling. Additionally, the separate dosage forms can be accessed and/or labeled for administration to the patient at different times.

In some embodiments, the kit may be a blister pack in which each separate dose of SNDX-275 and at least one other active pharmaceutical ingredient is segregated from the other doses in separate blisters or bubbles. In some such embodiments, the blister pack may have perforations, which allow each daily dose to be separated from the others by tearing it away from the rest of the blister pack. The separate dosage forms may be contained within separate blisters.

In some embodiments, the second active pharmaceutical ingredient may be in the form of a liquid or a reconstitutable powder, which may be separately sealed (e.g. in a vial or ampule) and then packaged along with a blister pack containing separate dosages of SNDX-275. This would be especially useful in a clinical setting where prescribed doses of SNDX-275 and a second active pharmaceutically active agent would be used on a dosing schedule in which SNDX-275 is administered on certain days and the second active pharmaceutical ingredient is administered on the same or different days within a weekly, biweekly, 2×weekly or other dosing schedule. Such a combination of blister pack containing SNDX-275 and one or more sealed containers of second active pharmaceutical ingredient could also include instructions for administering SNDX-275 and the second active pharmaceutical ingredient on a dosing schedule adapted to provide the synergistic or sequelae-treating effect of the second active pharmaceutical ingredient.

In other embodiments, the kit may be a container having separate compartments with separate lids adapted to be opened on a particular schedule. For example, a kit may comprise a box (or similar container) having seven compartments, each for a separate day of the week, and each compartment marked to indicate which day of the week it corresponds to. In some specific embodiments, each compartment is further subdivided to permit segregation of one active pharmaceutical ingredient from another. As stated above, such segregation is advantageous in that it prevents damage to the dosage forms and permits dosing at different times and labeling to that effect. Such a container could also include instructions for administering SNDX-275 and the second active pharmaceutical ingredient on a dosing schedule adapted to provide the synergistic or sequelae-treating effect of the second active pharmaceutical ingredient.

The kits may also include instructions teaching the use of the kit according to the various methods and approaches described herein. Such kits optionally include information, such as scientific literature references, package insert materials, clinical trial results, and/or summaries of these and the like, which indicate or establish the activities and/or advantages of the composition, and/or which describe dosing, administration, side effects, drug interactions, disease state for which the composition is to be administered, or other information useful to the health care provider. Such information may be based on the results of various studies, for example, studies using experimental animals involving in vivo models and studies based on human clinical trials. In various embodiments, the kits described herein can be provided, marketed and/or promoted to health providers, including physicians, nurses, pharmacists, formulary officials, and the like. Kits may, in some embodiments, be marketed directly to the consumer. In certain embodiments, the packaging material further comprises a container for housing the composition and optionally a label affixed to the container. The kit optionally comprises additional components, such as but not limited to syringes for administration of the composition.

In some embodiments, the kit comprises SNDX-275 that is visibly different from a second pharmaceutical composition. The visible differences may be for example shape, size, color, state (e.g. liquid/solid), physical markings (e.g. letters, numbers) and the like. In certain embodiments, the kit comprises SNDX-275 that is a first color and a second pharmaceutical composition that is a second color. In embodiments wherein the first and second colors are different, the different colors of the first and second pharmaceutical compositions is used, e.g., to distinguish between the first and second pharmaceutical compositions. In further embodiments, a third pharmaceutical composition is a third color.

In some embodiments, wherein the packaging material further comprises a container for housing the pharmaceutical composition, the kit comprises SNDX-275 that is in a different physical location within the kit from a second pharmaceutical composition. In some embodiments, the different physical locations of SNDX-275 and the second pharmaceutical compositions comprise separately sealed individual compartments. In certain embodiments, the kit comprises SNDX-275 that is in a first separately sealed individual compartment and a second pharmaceutical composition that is in a second separately sealed individual compartment. In embodiments wherein the SNDX-275 and second compartments are separate, the different locations are used, e.g., to distinguish between the SNDX-275 and second pharmaceutical compositions. In further embodiments, a third pharmaceutical composition is in a third physical location within the kit.

### Pharmacokinetics

In various embodiments, SNDX-275 is dosed so as to minimize toxicity to the patient. In some embodiments, SNDX-275 is dosed in a manner adapted to provide particular pharmacokinetic (PK) parameters in a human patient. In some embodiments, SNDX-275 is dosed in a manner adapted to provide a particular maximum blood concentration (Cₘₐₓ) of SNDX-275. In some embodiments, SNDX-275 is dosed in a manner adapted to provide a particular time (Tₘₐₓ) at which a maximum blood concentration of SNDX-275 is obtained. In some embodiments, SNDX-275 is dosed in a manner adapted to provide a particular area under the blood plasma concentration curve (AUC) for SNDX-275. In some embodiments, SNDX-275 is dosed in a manner to provide a particular clearance rate (CL/F) or a particular half-life (T_{1/2}) for SNDX-275. Unless otherwise specified herein, the PK parameters recited herein, including in the appended claims, refer to mean PK values for a cohort of at least 3 patients under the same dosing schedule. Thus, unless otherwise specified: AUC=mean AUC for a cohort of at least 3 patients; Cₘₐₓ=mean Cₘₐₓ for a cohort of at least 3 patients; Tₘₐₓ=mean Tₘₐₓ for a cohort of at least 3 patients; T_{1/2}=mean T_{1/2} for a cohort of at least 3 patients; and CL/F=mean CL/F for a cohort of at least 3 patients. In some embodiments, the mean is a cohort of at least 6 patients, or at least 12 patients or at least 24 patients or at least 36 patients. Where other than mean PK values are intended, it will be indicated that the value pertains to individuals only. Also, unless otherwise indicated herein, AUC refers to the mean AUC for the cohort of at least 3 patients, extrapolated to infinity following a standard clearance model. If AUC for a time certain is intended, the start (x) and end (y) times will be indicated by suffix appellation to “AUC” (e.g. AUC_{x, y}).

In some embodiments, SNDX-275 is dosed in a manner adapted to provide maximum blood concentration (Cₘₐₓ) of SNDX-275 of about 1 to about 135 ng/mL, especially about 1 to about 55 ng/mL, particularly about 1 to about 40 ng/mL of SNDX-275. In some embodiments, SNDX-275 is dosed in a manner adapted to provide maximum blood concentration (Cₘₐₓ) of SNDX-275 of about 1 to about 20 ng/mL, especially about 1 to about 10 ng/mL, particularly about 1 to about 5 ng/mL of SNDX-275. In some embodiments, SNDX-275 is dosed in a manner adapted to provide a Cₘₐₓ of 10-100 ng/mL. In various embodiments, the SNDX-275 is dosed in a manner adapted to provide a Cₘₐₓ of 10-75 ng/mL, or 10-50 ng/mL, or 10-25 ng/mL. In some embodiments, the SNDX-275 is dosed in a manner adapted to provide a Cₘₐₓ of less than about 50 ng/mL, or less than about 30 ng/mL, or less than about 20 ng/mL, or less than about 10 ng/mL, or less than about 5 ng/mL.

In some embodiments, SNDX-275 is dosed in a manner adapted to provide a particular time (Tₘₐₓ) of about 0.5 to about 24 h, especially about 1 to about 12 hours. In some embodiments, the Tₘₐₓ is greater than about 24 hours. In some embodiments, the Tₘₐₓ is less than about 6 hours. In some embodiments, the Tₘₐₓ is between about 30 minutes and about 24 hours. In various embodiments, the Tₘₐₓ is between about 30 minutes and about 6 hours. In some embodiments, the Tₘₐₓ is

In some embodiments, SNDX-275 is dosed in a manner adapted to provide a particular area under the blood plasma concentration curve (AUC) of SNDX-275 of about 100 to about 700 ng·h/mL. In some embodiments, SNDX-275 is dosed biweekly under conditions adapted to provide an AUC of about 190 to about 700 ng·h/mL of SNDX-275. In some embodiments, SNDX-275 is dosed weekly under conditions adapted to provide an AUC of about 200 to about 350 ng·h/mL. In some embodiments, SNDX-275 is dosed biweekly under conditions adapted to provide an AUC of about 100 to about 500 ng·h/mL. In some embodiments, SNDX-275 is dosed under conditions adapted to provide an AUC of about 75-225 ng·h/mL.

In some embodiments, the terminal half-life (T_{1/2}) of SNDX-275 is at least 48 hours. In some embodiments, the T_{1/2} is between about 48 hours and about 168 hours. In some embodiments, the T_{1/2} is between about 48 and 120 hours. In some embodiments, the T_{1/2} is between about 72 and 120 hours. In some embodiments, the T_{1/2} is between 24 and 48 hours.

In some embodiments, SNDX-275 is dosed in a manner adapted to provide a particular MRT of SNDX-275.

### EXAMPLES

The following non-limiting, illustrative examples provide further elucidation of the embodiments disclosed herein.

### Example 1

### Pharmacokinetics of SNDX-275 in Patients with Refractory Solid Tumors

SNDX-275 was administered orally to 3-6 fasting patients with refractory/relapsed solid tumors or lymphoma per dose level. The plasma PK profile of SNDX-275 was analyzed using a validated, quantitative method. Histone H3 and H4 acetylation was analyzed in peripheral blood mononuclear cells (PBMCs) by immunohistochemical detection.

### Aims and Objectives

This study was conducted to determine the maximum tolerated dose (MTD) of SNDX-275 in patients with refractory solid tumors and lymphomas, and to characterized the safety, toxicity, pharmacokinetics and antitumor activity of SNDX-275 in this patient population.

### Methods

Eligible patients had refractory solid tumors or lymphomas for which conventional therapies were not appropriate or were no longer beneficial. Patients were included in the study if they: were at least 18 years of age and had a life expectancy of greater than 3 months; had an Eastern Cooperative Oncology Group (ECOG) performance status of 2 or less, and had adequate bone marrow function (including ANC≧1500/μL; platelets≧100,000/μL) and organ function (including creatinine≦1.5×upper limit of normal (ULN); alanine and aspartate aminotransferases≦2.5×ULN).

### Treatment

This was an open-label study in which SNDX-275 was administered orally (PO) in tablet form using the following doses and schedules:

Schedule A: Biweekly dosing: 14-day treatment cycle with SNDX-275 administered at a dosage of 2, 4, 6, 8 or 10 mg/m² on Day 1, followed by 13 days of rest.

Schedule B: Twice-weekly dosing: 4-week treatment cycle; SNDX-275 was administered at a dosage of 2, 3, 4 or 5 mg/m², administered twice weekly during Weeks 1, 2 and 3 of the 4-week cycle.

Schedule C: Once-Weekly dosing: 4-week treatment cycle; SNDX-275 was administered once weekly for Weeks 1, 2 and 3 of the 4-week cycle.

Three patients were enrolled at each dose level, with additional three patients enrolled if dose-limiting toxicity (DLT) was observed in at least one patient. The dosing schedule is summarized in Table 1-1, below.

TABLE 1-1
SNDX-275 Study Dosing Schedule

| | Schedule A | Schedule B | Schedule 3 |
|---|---|---|---|
| | Biweekly dosing | Twice weekly dosing | Weekly dosing |
| | (n = 10) | (n = 6) | (n = 8) |
| Male/female (n = 24) | 10/0 | 3/3 | 2/6 |
| Median age, years (range) | 61.5 (24-76) | 57 (45-79) | 51 (40-78) |
| Tumor type | melanoma (3), | melanoma (1), | melanoma (1), |
| | colon (1), prostate (1), | colon (2), GIST (2), | prostate (1), breast (2), |
| | adrenocortical (1), | sarcoma (1) | renal cell carcinoma (1), |
| | carcinoid (1), rectal (1), | | NSCLC (1), |
| | Ewing's sarcoma (1), | | leiomyosarcoma (1), colon |
| | mesothelioma (1) | | (1) |

GIST, gastrointestinal stromal tumor;
NSCLC, non-small cell lung cancer

### Tolerability Assessments

Tolerability was assessed by scheduled clinical assessments. Adverse events (AEs) were assessed using the National Cancer Institute Common Toxicity Criteria (CTC) version 2.0. DLT was defined as: (1) Any grade 4 hematologic toxicity; (2) grade 3 or greater non-hematologic toxicity (excluding alopecia and nausea/vomiting in patients not receiving maximal supportive treatment); (3) grade 2 non-hematologic toxicity (excluding alopecia) intolerable to the patient or of concern to the investigator that interrupted the dosing cycle or did not return to grade 1 or pretreatment baseline by the time of the next scheduled treatment cycle. MTD was defined as the highest dose at which no more than 1 of 6 patients experienced DLT during the first treatment cycle.

### Activity

Tumor response was assessed every 6 weeks on the biweekly schedule (Schedule A) and every 8 weeks on the twice-weekly (Schedule B) and weekly (Schedule C) schedules, using appropriate imaging studies. Tumor responses were defined by the Response Evaluation Criteria on Solid Tumors.

### Pharmacokinetics

Blood samples (5 ml) were taken at the following times:

Schedule A: Before treatment and at 1, 2, 3, 4, 6, 8, 12, 24, 36, 48, 60, 72, 84, 96, 120 and 168 hours post treatment after the first dose of SNDX-275.

Schedule B: Before treatment and at 0.5, 1, 1.5, 2, 4, 8, 12, 24, 48 and 72 or 96 hours post treatment with the first and sixth dose of SNDX-275.

Schedule C: Before treatment and at 0.25, 0.5, 1, 1.5, 2, 4, 8, 24, 48 and 96 hours post-treatment after the first and third dose of SNDX-275 and 168 hours after the third dose.

Blood was centrifuged for 10 minutes at 2000 g at room temperature within approximately 30 minutes after withdrawal; plasma was separated and stored at −18° C. to −25° C. until analysis. For analysis, SNDX-275 was extracted from the plasma samples using ethyl acetate; the supernatant was separated and the solvent evaporated under nitrogen. The residue was reconstituted in mobile phase and plasma concentration of SNDX-275 was examined using a validated, quantitative liquid chromatography-mass spectrometer (LC-MS) method using a 1100 LC-MSD (Agilent Technologies, Foster City, USA). The precision and accuracy of the assay was determined by using human plasma samples prepared with concentrations of SNDX-275 of 0.5, 1.0, 3.0, 40 and 80 ng/mL.

### Results

Ten (10) patients were enrolled on Schedule A, 6 on Schedule B, 8 on Schedule C. See Table 1-1. The median number of weeks (range) on study was 7 (3-86) on Schedule A, 9.5 (5-42) on Schedule B and 8 (2-13) on Schedule C.

Pharmacokinetics

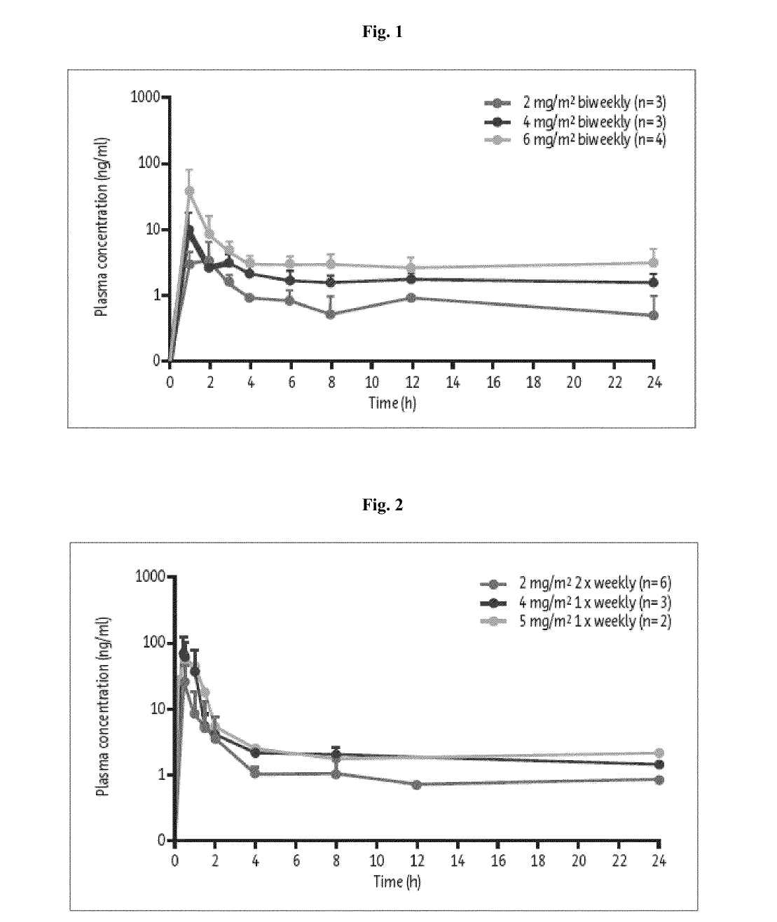

SNDX-275 was rapidly absorbed under fasting conditions with a Tₘₐₓ within 60 minutes of treatment. See FIGS. 1 and 2. FIG. 1 shows the mean plasma concentration over time for SNDX-275 (2, 4, 6 mg/m²) after the biweekly dose on Schedule A. FIG. 2 shows the mean plasma concentration over time for SNDX-275 (2 mg/m²) twice weekly on Schedule B and SNDX-275 (4 and 5 mg/m²) weekly on Schedule C. As can be seen from FIGS. 1 and 2, the apparent drug clearance (CL/F) of SNDX-275 was low and consequently a long T_{1/2} of approximately 100 hours was observed. A moderate 1.3-1.5 fold serum accumulation of SNDX-275 was observed over a single treatment cycle consisting of one or two doses per week for 3 weeks.

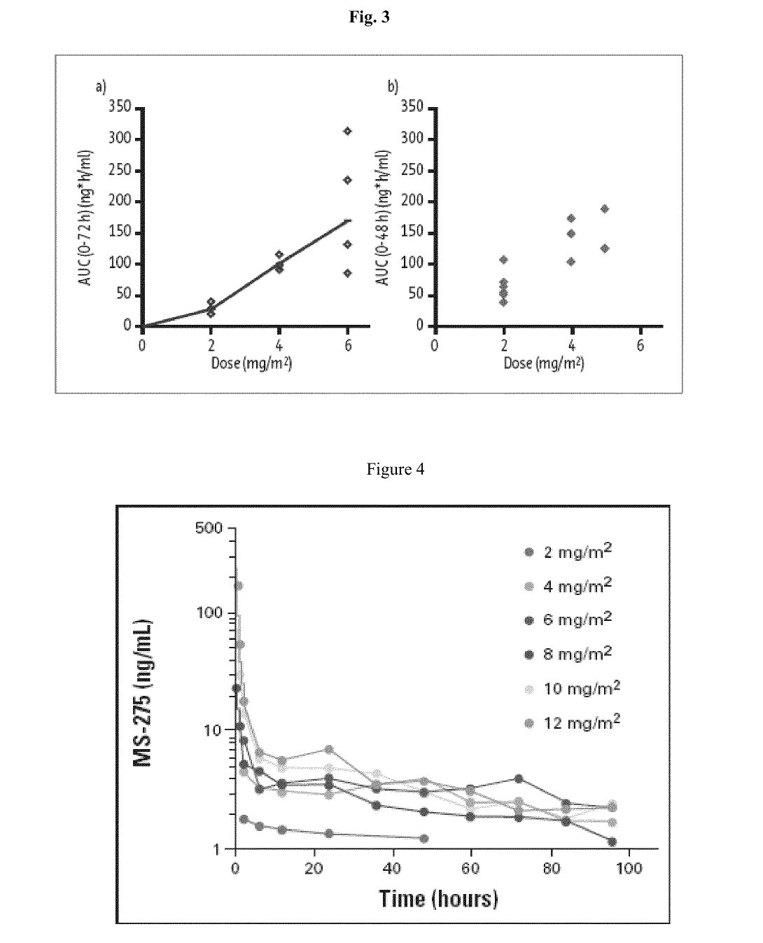

FIG. 3a) shows that there is dose proportionality for SNDX-275 on Schedule A after the first dose at dosages of 2, 4 and 6 mg/m² (AUC_{0 Tmax}); and FIG. 3b) shows a similar dose proportionality for SNDX-275 on schedules B and C after the first dose at dosages of 2, 4 and 5 mg/m². A dose-linear and dose-dependent increase in systemic exposure was produced with SNDX-275 administered biweekly (Schedule A, FIG. 3a). Primary pharmacokinetic data are summarized in Table 1-2.

TABLE 1-2
Summary of PK Data From the Study

| | Schedule B | Schedule 3 | | | | | | | |
|---|---|---|---|---|---|---|---|---|---|
| | Biweekly | Twice weekly dosing | | | | Weekly dosing | | | |
| | dosing | (2 mg/m²) | | | | (5 mg/m²) | | | |
| Schedule A | (6 mg/m²) | Day 1 | | Day 15 | | Day 1 | | Day 22 | |
| Cₘₐₓ (ng/ml) | nd | 25.3 | (14.8-66.4) | 12.8 | (6.5-26.5) | 72.0 | (58.3-89.0) | 130 | (104-162) |
| Tₘₐₓ (h) | nd | 0.5 | (0.5-1.0) | 0.5 | (0.5-0.5) | 0.25 | (0.25-0.25) | 0.75 | (0.5-1.0) |

| | | | | | | | | | |
|---|---|---|---|---|---|---|---|---|---|
| T_{1/2} (h) | 102 | (83-139) | nd | 100 | (59-135) | 112 | (65-193) | 77 | (75-79) |

| | | | | | | | | | |
|---|---|---|---|---|---|---|---|---|---|
| AUC₍₀₋₄₈₎ (ng * h/ml) | 170 | (85.9-313) | 60.1 | (37.8-106) | 85.9 | (50.8-161) | 153 | (124-188) | 205 |

| | | | | | |
|---|---|---|---|---|---|
| AUC (ng * h/ml) | 412 | (199-793) | nd | 432 | (391-478) |
| Cl/f (l/h/m²) | 14.5 | (7.6-30.1) | nd | 11.6 | (10.5-12.8) |

### Clinical Activity

One sustained partial response has been observed in a patient with melanoma on Schedule A, who had received SNDX-275 for over 86 weeks (Table 1-3). Stable disease has been reported in 7 patients, including 2 patients with melanoma for 12 to 52 weeks; one patient with melanoma continued to receive therapy after completion of the study (Table 1-3).

TABLE 1-3
Summary of SNDX-275 Responses

| | | | |
|---|---|---|---|
| Patient Number | Tumor Type | Weeks on Study | Best Response |

Schedule A

| | | | |
|---|---|---|---|
| 1 | melanoma | 86* | PR |
| 2 | rectal carcinoma | 35 | SD |
| 3 | prostate | 7 | PD |
| 4 | carcinoid | 4 | PD |
| 5 | mesothelioma | 5 | PD |
| 6 | Ewing's sarcoma | 52 | SD |
| 7 | adenocortical | 6 | na |
| 8 | melanoma | 3 | PD |
| 9 | melanoma | 7 | PD |
| 10 | colon | 13 | SD |

Schedule B

| | | | |
|---|---|---|---|
| 11 | colon | 8 | PD |
| 12 | melanoma | 42 | SD |
| 13 | colon | 11 | PD |
| 14 | sarcoma | 13 | PD |
| 15 | GIST | 5 | PD |
| 17 | GIST | 8 | PD |

Schedule C

| | | | |
|---|---|---|---|
| 18 | breast | 8 | PD |
| 19 | NSCLC | 12 | SD |
| 20 | prostate | 13 | SD |
| 21 | renal cell | 8 | n/a |
| 22 | melanoma | 12 | SD |
| 24 | leiomyosarcoma | 2 | n/a |
| 25 | colon | 4 | n/a |
| 26 | breast | 3 | n/a |

### Conclusions

SNDX-275 was well-tolerated when administered biweekly at doss up to 6 mg/m² (Schedule A); dose escalation beyond 2 mg/m² was not pursued on the twice-weekly schedule (Schedule B). Two DLTs were observed at 5 mg/m² weekly (schedule C) and additional patients are being enrolled at 4 mg/m². PK data suggest a rapid absorption of SNDX-275 under fasting conditions and subsequently a rapid distribution of SNDX-275 into deep tissue compartments, with a longer persistence occurring within the deeper compartments. SNDX-275 was found to produce a dose-linear and dose-dependent increase in systemic exposure. A partial response was seen in one melanoma patient who continued to receive SNDX-275 for a minimum of 22 months.

Seventeen (17) patients have been enrolled in three schedules. Schedule A: 2-6 mg/m² biweekly (N=10); Schedule B: 2 mg/m² twice weekly with 1 week rest (N=6); Schedule C: 4 mg/m² (N=1). Eighty cycles have been administered on Schedule A and 13 on Schedule B. No drug-related grade 4 (gr4) adverse events (AEs) were reported. Maximum tolerated dose (MTD) was not reached on Schedule A. The plasma profile SNDX-275 demonstrates rapid absorption with a Tₘₐₓ of 0.5-2.0 hours and a dose-dependent increase in systemic exposure over the dose range 2-6 mg/m². A biphasic elimination was noted with an estimated T_{1/2} of 100 hours. Preliminary PO analyses indicate an increase in histone H3 and H4 acetylation in PBMCs, compared with pretreatment. One patient with melanoma continued to exhibit a partial response (Schedule A) and one patient each with Ewing's sarcoma (Schedule A), rectal carcinoma (Schedule A) and melanoma (Schedule B) had stable disease after 60+, 38+ and 20+ weeks of therapy, respectively. The PK data by dosing schedule are summarized in Table 1-4, below.

TABLE 1-4
PK Parameters by Dosing Schedule

| SNDX-275 | | | Mean (SD) | Mean (SD) | Mean (SD) | | Median |
|---|---|---|---|---|---|---|---|
| Dose | | | Cₘₐₓ | AUC | CL/F | Mean (SD) | (range) |
| mg/m² | Schedule | No. Patients | (ng/mL) | (ng · h/mL) | (L/h) | t_{1/2} (h) | Tₘₐₓ (h) |
| 2 | biweekly | 3 | 4.48 | 107 | 20.7 | 93.7 | 1 |
| | | | (2.38) | (47.0) | (9.08) | (45.9) | (1, 2) |
| 4 | biweekly | 3 | 9.42 | 373 | 11.3 | 161 | 1 |
| | | | (7.94) | (96.8) | (3.17) | (73.8) | (1, 1) |
| 6 | biweekly | 4 | 37.3 | 459 | 16.7 | 107 | 1 |
| | | | (38.0) | (241) | (9.82) | (22.7) | (1, 2) |
| 2 | 2 × | 6 | 29.1 | 135 | 15.4 | 54.0 | 0.6 |
| | weekly | | (19.1) | (31.2) | (3.05) | (17.0) | (0.5, 1) |
| 4 | weekly | 6 | 75.3 | 323 | 13 | 87.9 | 0.5 |
| | | | (40.2) | (67.0) | (3.46) | (30.3) | (0.25, 1) |
| 5 | weekly | 5 | 77.3 | 494 | 10.7 | 133 | 0.5 |
| | | | (28.4) | (141) | (2.51) | (61.3) | (0.5, 1) |

### Example 2

### Pharmacokinetic Study of SNDX-275 in Patients with Advanced and Refractory Solid Tumors or Lymphoma

The purpose of this study was to define the maximum-tolerated dose (MTD), the recommended phase II dose, the dose-limiting toxicity, and determine the pharmacokinetic (PK) and pharmacodynamic profiles of SNDX-275. Patients with advanced solid tumors or lymphomas were treated with SNDX-275 orally initially on a once-a-day×28 every 6 weeks (daily) and later on once-every-14-days (q14-day) schedules. The starting dose was 2 mg/m² and the dose was escalated in three- to six-patient cohorts based on toxicity assessments. With the daily schedule, the MTD was exceeded at the first dose level. Preliminary PK analysis suggested the half-life of SNDX-275 in humans was 39 to 80 hours, substantially longer than predicted by preclinical studies. With the q14-day schedule, 28 patients were treated. The MTD was 10 mg/m² and dose-limiting toxicities were nausea, vomiting, anorexia and fatigue. Exposure to SNDX-275 was dose-dependent, suggesting linear PK. Increased histone H3 acetylation in peripheral-blood mononuclear-cells was apparent at all dose levels by immunofluoresence analysis. Ten of 29 patients remained on treatment for greater than 3 months. Thus, the SNDX-275 oral formulation on the daily schedule was intolerable at a dose and schedule explored. The q14-day schedule is reasonably well-tolerated. Histone deacetylase inhibition was observed in peripheral-blood mononuclear-cells.

### Patients

Inclusion criteria were as follows: Pathologically confirmed malignancy that was metastatic or unresectable and for which standard curative or palliative measure did not exist or would likely not be effective; (2) an Eastern Cooperative Oncology Group (ECOG) performance status≦2, with no recent (within 2 months) weight loss of >10% of average body weight; (3) life expectancy greater than 3 months; (4) age≧18 years; (5) leukocytes≧3,000/μL, platelets≧100,000/μL, creatinine within normal limits or measured creatinine clearance≧60 mL/min/1.73 m², total bilirubin≦1.5×upper limit of normal, AST/ALT≦2.5×upper limit of normal, adequate oral intake and serum albumin>75% of lower limit of normal; and (6) tablet to give written consent, willing to self-administer and document doses of SNDX-275 as needed, and able to return to the study center for follow-up. The demographics of the patient cohort are summarized in Table 2-1, below.

TABLE 2-1
Patient Characteristics

| Characteristic | No. of Patients |
|---|---|
| Total | 31 |
| Age, years | |
| Median: 57 | |
| Range: 36-76 | |
| Sex | |
| Male | 19 |
| Female | 12 |
| ECOG performance status | |
| 0 | 7 |
| 1 | 21 |
| 2 | 3 |
| Median | 1 |
| Tumor type | |
| Melanoma | 6 |
| Renal cell carcinoma | 6 |
| NSCLC | 4 |
| Sarcoma | 4 |
| Breast | 2 |
| Colorectal | 2 |
| Lymphoma | 2 |
| Cervix | 1 |
| Mesothelioma | 1 |
| Prostate | 1 |
| Small Bowel | 1 |
| Thyroid | 1 |
| No. of prior chemotherapy | |
| 0 | 1 |
| 1 | 6 |
| 2 | 10 |
| ≧3 | 14 |
| Median | 3 |
| Range | 0-20 |
| No. of prior radiotherapy | |
| 0 | 16 |
| 1 | 9 |
| 2 | 4 |
| ≧3 | 2 |
| No. of prior immunotherapy | |
| 0 | 16 |
| 1 | 9 |
| 2 | 6 |

Exclusion criteria were as follows: (1) those who had received prior anticancer therapy (chemotherapy, radiotherapy, vaccines, and hormone therapy with the exception of gonadotropin hormone-releasing hormone agonists) within 4 weeks of study entry (6 weeks for nitrosoureas or mitomycin C, 8 weeks for UCN-01) or those who have not recovered from adverse events (reduced to grade 2 or less) as a result of agents administered more than 4 weeks earlier; (2) known brain metastases; (3) history of allergic reactions attributed to compounds of similar chemical or biologic compositions to SNDX-275; (4) uncontrolled intercurrent illness; (5) pregnant or lactating women; (6) men and women of reproductive potential without adequate contraception; (7) known HIV; (8) gastrointestinal conditions that might predispose for drug intolerability or poor drug absorption; and (9) major surgery within 21 days of study entry, intercurrent radiation, chemotherapy, immunotherapy, or hormonal therapy (except for gonadotropin hormone-releasing hormone agonists).

### Dosage and Dose Escalation Scheme

The initial dosing schedule was daily oral administration for 28 days and 14-day recovery period, constituting a 42-day cycle. SNDX-275 was administered with food, owing to evidence of enhanced bioavailability from animal studies in the fed state. A starting dose of 2 mg/m² ( 1/10^{th} of rat MTD) with an accelerated dose escalation at increments of 100% and single patient per dose level was planed.

Due to unexpected toxicities, the subsequent dosing schedule was changed to once orally every 14 days. Administered in the fed state, the starting dose level was again 2 mg/m², using a modified Fibonacci dose escalation scheme (three to six patient cohorts) with a dose escalation increment of 2 mg/m² without intrapatient dose escalation.

DLT was defined as first course adverse events ≦grade 3 nonhematologic or ≦grade 4 hematologic toxicity. The MTD was defined as one dose level below the dose at which ≦two of six patients experienced DLT.

Dose reduction by one level was applied for the occurrence of either grade 3 nonhematologic toxicity, grade 4 hematologic toxicity, persistent (≦2 weeks) grade 2 nonhematologic toxicity, or per the investigator's assessment. For dose level 1, 25%, 50% and 75% decrease in starting dose was the order of dose reduction. No limitation for the number of dose reductions was chosen. The dosing schedule is summarized in Table 2-2, below.

TABLE 2-2
Dosing Schedule

| | | | | | |
|---|---|---|---|---|---|
| Dose Level | | Initial | Total No. of | No. of Patients | |
| and | Dose | Patient | Treatment | With First | |
| Schedule | (mg/m²) | No. | Courses | Course DLT | DLTs |

Every day x 28/42 days

| | | | | | |
|---|---|---|---|---|---|
| 1 | 2 | 2 | 2* | 2 | See text |

Every 14 days

| | | | | | |
|---|---|---|---|---|---|
| 1 | 2 | 3 | 22 (4) | 0 | 0 |
| 2 | 4 | 3 | 16 (4) | 0 | 0 |
| 3 | 6 | 6 | 51 (8) | 1 | 3† |
| 4 | 8 | 5 | 22 (9) | 0 | 0 |
| 5 | 10 | 6 | 30 (8) | 1 | 3† |
| 6 | 12 | 5 | 16 (5) | 2 | 7‡ |

NOTE.
Numbers in parentheses indicate total patients treated at dose level.
Abbreviation:
DLT, dose-limiting toxicity.
*Due to DLTs, both patients' treatments were terminated before completing the first course.
†Anorexia, nausea, and vomiting.
‡Anorexia, nausea, vomiting, and fatigue.

### Safety and Efficacy Measures

At study entry, history, physical examination, laboratory studies (CBC, electrolytes, creatinine, blood urea nitrogen, total and direct bilirubin, ALT, AST, alkaline phosphatase, uric acid, prothrombin time, partial thromboplastin time, and urinalysis), computed tomography scan and chest x-ray and ECG were performed. Clinical assessments, including a physical examination and adverse event evaluation, were conducted at each follow-up. Adverse events were graded by the NCI Common Toxicity Criteria (version 2.0). Computed tomography scans and staging was performed every 6 weeks for the q14-day schedule. Disease-specific staging techniques, such as bone marrow aspirate and biopsy, flow cytometry, cutaneous lesion photography, or bone scan were used as indicated. Response evaluations used the Response Evaluation Criteria in Solid Tumors and the Cheson criteria for lymphoma. Multiple-gated acquisition (MUGA) scans were obtained on the q14-day schedule at base line, before course 2 and at each restaging. Laboratory studies (CBC with differential, chemistry 20, prothrombin time and particle thromboplastin time) were performed on days 1, 3, 5, 7 and repeated weakly. Twenty-four-hour urine clearance, albumin, protein, uric acid and electrolytes were performed at baseline and on days 3 and 13.

### Pharmacokinetic (PK) Studies

Blood samples for pharmacokinetic studies (6 mL each) were collected in tubes containing sodium heparin at the following time points: 0, 2, 6, 12, 24, 48, 60, 7, 84 and 96 hours after first dose of SNDX-275. Following initial PK evaluation of data obtained from the first two dose levels, the sampling also included 30 minutes and 1 hour. Samples were immediately centrifuged at 3000 g for 10 minutes at 4° C. and then plasma was divided into two aliquots of at least 1 mL and frozen at −70° C. until the time of analysis. Plasma samples were assayed by a specific and sensitive high-performance liquid chromatographic assay with mass-spectrometric detection. The lower limit of quantitation of this assay is 0.50 ng/mL, with values for precision and accuracy of ≦5.58 and ≦11.4% relative error, respectively.

Estimates of pharmacokinetic parameters for SNDX-275 were derived from individual concentration-time data sets by non-compartmental analysis using the software package WinNonlin version 4.0 (Pharsight Corporation, Mountain View, Calif.). The pharmacokinetic parameters of interest included peak plasma concentration (Cmax), time to peak concentration (Tmax), area under the plasma concentration versus time curve extrapolated to infinity (AUC), apparent oral clearance (CL/F), and the terminal half-life (T_{1/2, z}). The peak plasma concentrations (Cmax) and the time to peak plasma concentrations (Tmax) were the observed values. The area under the plasma concentration versus time curve (AUC) was calculated using the linear trapezoidal method from time zero to the time of the final quantifiable concentration (AUC_{tf}). The AUC was then extrapolated to infinity (AUC_{inf}) by dividing the last measured concentration by the rate constant of the terminal phase (k), which was determined by linear-regression analysis of the final three or four time points of the ling-linear concentration time plot. The apparent oral clearance of SNDX-275 (CL/F) was calculated by dividing the administered dose by the observed AUC_{inf} and the T_{1/2} was calculated by dividing 0.693 by k.

Dose proportionality for SNDX-275 was assessed using a power model (i.e. AUC=α×dose^{β}) where an ideal proportional model corresponds to β=1 (i.e., to a model of the form AUC=α×dose) and with the proportionality constant α. Deviations of β from 1 correspond to deviations from ideal dose proportionality. Interindividual differences in PK parameters were assessed by the coefficient of variation (CV), expressed as the ratio of standard deviation to the observed mean (SD/M). All PK data are presented as mean±SD, except where otherwise indicated. The apparent CL/F and the T_{1/2} were analyzed as a function of the SNDX-275 dose level using the Kruskal-Wallis' one-way analysis of ranks followed by the Dunn's multiple comparison test for identifying statistically significantly different groups. Variability in parameter estimates for SNDX-275 between cohorts of patients that did or did not experience DLT was evaluated by a one-sided Man-Whitney U test for differences in medians after testing for normality and heteroscedasticity. One-way analysis of variance was performed to compare mean values using a two-sided Dunnett's test. Statistical calculations were performed using the Number Cruncher Statistical System 2001 series (J. L. Hintze, Kaysville, Utah). The cut-off for statistical significance was considered at P<0.05.

### Pharmacodynamic Analysis

Immunocytochemical analysis of acetylated histone H3 was performed on peripheral-blood mononuclear cells (PBMCs), which were isolated from whole blood by centrifugation on Ficoll-Paque Plus (Amersham, Little Chalfont, United Kingdom), pelleted onto glass slides by centrifugation, fixed in 95% ethanol/5% glacial acetic acid for 1 minute and permeabilized with 0.2% Triton X-100 for 10 minutes at room temperature, then non-specific binding sites were blocked by incubating the cells with 1% bovine serum albumin in phosphate-buffered saline (PBS) for 1 hour at 4° C. Slides were incubated with polyclonal antiacetylated histone H3 antibody (Upstate Biotechnology, Lake Placid, N.Y.) of 1 hour at 4° C., washed two times for 2 minutes with PBS, then incubated at 4° C. for 1 hour with Cy3-conjugated goat antirabitt immunoglobulin (Molecular Probes, Eugene, Oreg.), and washed again with PBS. Finally, slides were incubated with 4,6-diamidoino-2-phenylindole (Sigma, St. Louis, Mo.) for 10 minutes at room temperature, rinsed quickly with water, air-dried, mounted using SlowFade (Molecular Probes), and imaged using a Zeiss Axiophot microscope interfaced with a CCD camera (Optronics Engineering, Goleta, Calif.). Positive controls were prepared by exposing healthy donor PBMCs to SNDX-275 in vitro. Buffy coats, provided anonymously as a byproduct of whole-blood donations from paid, healthy volunteer donors through an international review board-approved protocol, were centrifuged on Ficoll-Paque Plus. Mononuclear cells were depleted of monocytes by adherence to plastic for 2 hours at 37° C. and incubated with SNDX-275 in vitro for various times and at varying drug concentrations. Cells were processed for histone hyperacetylation in the same manner as patient samples. Images of PBMCs stained for acetylated histone H3 were imported into the Openlab image analysis program (Improvision, Coventry, United Kingdom) and histone acetylation levels were assessed using the Openlab quantification software.

### Results

### General

This study considers the results obtained from a cohort of 31 patients (two on daily and 29 on q14-day schedules.) Thirty patients received SNDX-275 and were assessable. One patient with melanoma withdrew before receiving treatment owing to a disease complication. All patients (demographic characteristics of the enrolled cohort are shown in Table 3-1) had received prior therapy (median No. of prior treatments=3): surgery (90%), prior chemotherapy (97%), radiotherapy (50%), and immunotherapy (50%).

Dose Escalation and DLT in Daily and q14-Day Schedule

The dose escalation experience for both the SNDX-275 daily and the q14-day schedules are summarized in Table 3-2.

Daily Schedule: Two male patients were treated at the initial dose level of 2 mg/m² of the daily×28 schedule. Both experienced DLT before the completion of the first cycle. DLTs observed were abdominal/epigastric pain in one patient and cardiac arrhythmia (supraventricular tachycardia), elevated AST/ALT, hypotension, hypoalbuminemia and hypophosphatemia in a second patient. All adverse events resolved within 2 to 3 weeks. Preliminary pharmacokinetic data from the initial two patients suggested that SNDX-275 had a 30- to 50-times longer half-life in humans than initially predicted from the animal models. This may explain the unforeseen toxicity observed in these two patients during the daily SNDX-275 schedule. Assessment of histone H3 and H4 acetylation indicated HDAC inhibition occurred after one dose of SNDX-275. To ensure safety, a q14-day dosing schedule was implemented.

q14-Day Dosing: A total of 28 patients were treated on the q14-day schedule. The DLTs of SNDX-275 on a q14-day schedule were anorexia, nausea, vomiting and fatigue. The MTD and recommended phase II dose of SNDX-275 for a q14-day schedule was 10 mg/m². As summarized in Table 3-2, the first patients with first course DLTs were observed at dose level 3 (6 mg/m²). After five patients tolerated dose level 4 without DLT, dose escalation continued to level 5 (10 mg/m²). One patient experienced similar DLTs at level 5 as had been seen at level 3. At dose level 6 (12 mg/m²), two patient experienced similar DLTs.

First course adverse events observed, either probably or possibly related to SNDX-275 are summarized in Table 2-3. There were no SNDX-275-related first course grade 4 adverse events. There was only one first course grade 4 adverse event (dyspnea) observed during the study, which occurred at dose level 6 (12 mg/m²), which was considered unrelated to SNDX-275, and likely was due to progression of metastatic mesothelioma. SNDX-275-induced fatigue, anorexia, nausea and vomiting were observed as early as dose level 1 (2 mg/m²), and all were mild. With dose escalation, intensity of these toxicities gradually increased. Other less frequent drug-related toxicities included taste change, headache, diarrhea, flatulence, bloating and reflux symptoms. Hematologic toxicities, such as thrombocytopenia and neutropenia, became more apparent at the higher dose levels. (Table 3-3). Anemia was frequently noticed during the first course due to frequent PK and laboratory sampling not related to SNDX-275.

TABLE 2-3
Adverse Events

All Grades

| Adverse Events | No. of Patients | % | Grade 3 |
|---|---|---|---|
| Gardiovascular | | | |
| Sinus Tachycardia | 1 | 3 | |
| Hematologic | | | |
| Anemia | 8 | 29 | |
| Leucopenia | 6 | 21 | |
| Lymphopenia | 5 | 18 | |
| Neutropenia | 7 | 25 | |
| Thrombooytopenia | 10 | 36 | |
| Gastrointestinal | | | |
| Anorexia | 10 | 36 | 4 |
| Constipation | 2 | 7 | |
| Diarrhea | 2 | 7 | |
| Dyspepsia | 6 | 21 | |
| Flatulence | 3 | 11 | |
| GI other | 2 | 7 | |
| Nausea | 18 | 64 | 4 |
| Stomatitis | 1 | 4 | |
| Vomiting | 11 | 39 | 4 |
| Laboratory | | | |
| Alkaline phosphatase | 1 | 4 | |
| Bilirutin | 4 | 14 | |
| Creatinine | 2 | 7 | |
| Hyperglycemia | 3 | 11 | |
| Hypermagnesemia | 2 | 7 | |
| Hypoalbuminemia | 18 | 64 | |
| Hypocalcemia | 6 | 21 | |
| Hypokalemia | 1 | 4 | |
| Hyponatremia | 7 | 25 | |
| Urinary electrolyte wasting | 3 | 11 | |
| General | | | |
| Allergic reaction | 1 | 4 | 0 |
| Dehydration | 3 | 11 | 0 |
| Depression | 1 | 4 | 0 |
| Fatigue | 15 | 54 | 1 |
| Fever | 1 | 4 | 0 |
| Headache | 14 | 50 | 0 |
| Infection w/o neutropenia | 2 | 7 | 0 |
| Libido | 1 | 4 | 0 |
| Middle ear infection | 1 | 4 | 0 |
| Muscle weakness | 1 | 4 | 0 |
| Myalgia | 1 | 4 | 0 |
| Nail changes | 1 | 4 | 0 |
| Sweating | 1 | 4 | 0 |
| Taste disturbance | 8 | 29 | 0 |
| Neuromuscular | | | |
| Neurosensory deficits | 2 | 7 | |
| Tremors | 1 | 4 | |
| Pain | | | |
| Abdominal pain | 2 | 7 | |
| Chest pain | 2 | 7 | |
| Pain other | 1 | 4 | |
| Pleuretic pain | 1 | 4 | |
| Respiratory | | | |
| Cough | 1 | 4 | |
| Rhinitis | 1 | 4 | |

### Pharmacokinetics

Pharmacokinetic studies were performed in 28 patients with complete concentration-time profiles available for 27 patients. FIG. 4 shows the plasma concentration versus time profile of SNDX-275 were very similar at each dose level. The mean non-compartmental PK parameters of SNDX-275 ranging from 2 to 12 mg/m² are summarized in Table 2-4. Substantial interpatient variability in PK parameters was apparent at any dose level (CV for AUC, up to 53%). Similar variability was apparent in the CL/F (CV=38.8%), implying varied systemic exposure to SNDX-275 during drug treatment. Absorption of the drug was highly variable with median Tmax approaching 2 hours, with slow gastrointestinal uptake of SNDX-275 resulting in a Tmax at 24 hours (n=2), 48 hours (n=1), and even 60 hours (n=1), whereas a few patients exhibited Tmax at 0.5 hours (n=7), suggesting a rapid absorption and possible underestimation of the extent of drug uptake in these individuals.

TABLE 2-4
Summary of Non-Compartmental PK Parameters

| | | Cₘₐₓ | | AUC (ng- | | CL/f | | | | | |
|---|---|---|---|---|---|---|---|---|---|---|---|
| | No. of | (ng/mL) | | h/mL) | | (1/h/M²) | | t_{1/2} (hours) | | Tₘₐₓ | |
| Dose (mg/m²) | Patients | Mean | SD | Mean | SD | Mean | SD | Mean | SD | Median | Range |
| 2 | 3 | 1.72 | 0.23 | 196.26 | 104.5 | 13.77 | 10.27 | 80.20 | 48.68 | 6 | 2-24 |
| 4 | 3 | 4.84 | 1.10 | 391.68 | 150.71 | 11.33 | 4.57 | 50.51 | 12.96 | 6 | 2-36 |
| 6 | 6 | 9.59 | 4.57 | 492.81 | 177.77 | 13.18 | 3.43 | 52.78 | 20.25 | 2 | 2-60 |
| 8 | 5 | 15.49 | 11.65 | 357.71 | 38.14 | 22.58 | 2.71 | 39.73 | 15.23 | 2 | 0.5-24 |
| 10 | 6 | 45.07 | 59.34 | 528.87 | 170.57 | 20.50 | 5.99 | 51.58 | 10.49 | 1.5 | 0.5-2 |
| 12 | 4 | 131.63 | 128.3 | 680.16 | 262.0 | 19.85 | 8.01 | 45.00 | 6.53 | 0.5 | 0.5-2 |
| Grand mean | | | | | | 17.40 | 6.75* | 51.74 | 21.55† | | |
| Grand | | | | | | | | | | 1.75 | 0.5-60 |
| median | | | | | | | | | | | |

Disappearance of SNDX-275 from the central plasma compartment was characterized by an apparent bi-exponential decline, with an overall slow apparent CL/F of 17.4±6.75 L/h/m². The estimated apparent terminal half-life was similar for the group of patients, exhibiting a mean value of 51.74±21.55 hours (CV=41.7%). As a result of the slow clearance, SNDX-275 was detectable even 5 days after initial treatment in 19 of 27 patients.

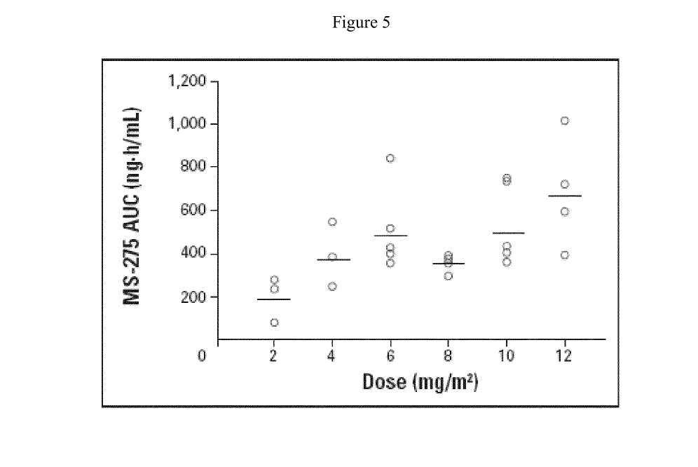

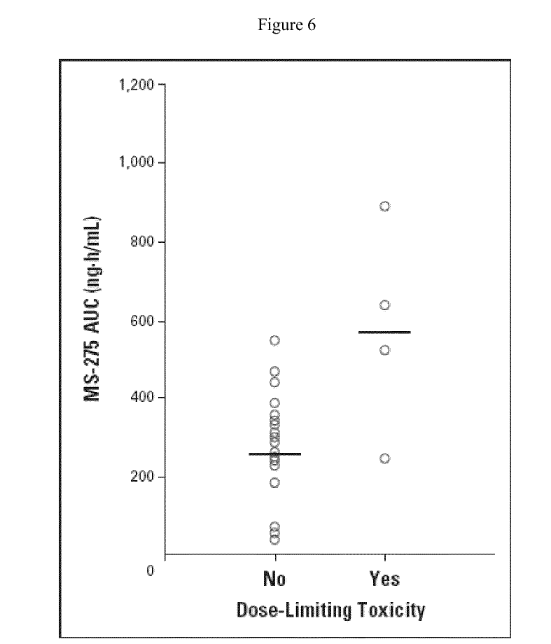

The peak plasma concentrations, as well as the AUCs, increased in near proportion with increasing doses of SNDX-275. (FIG. 5.) The power model analysis indicated that the model poorly described the data, which estimates the parameter β was 0.517±0.172 (R²−0.323), while linear regression analysis indicated near dose proportionality (R²=0.556). The mean apparent CL/F of SNDX-275 was not significantly dependent on drug dose (P=0.071) and the estimated T_{1/2} was dose independent (P=0.652). A preliminary analysis of pharmacokinetic-pharmacodynamic relationships for SNDX-275 suggests that drug exposure is significantly higher in patients experiencing DLTs (mean AUC, 517±276 ng·h/mL, n=4) compared with patients that had not DLT (280±121 ng·h/mL, n=23; P=0.0477; FIG. 6).

### Analysis

These data indicate that SNDX-275 can be given safely on a q14-day schedule, but not on a daily schedule in the dose range explored. Unlike the daily schedule, the q14-day schedule had neither symptomatic nor diagnostic cardiac adverse events observed. The low to medium dose range of 2 to 4 mg/m² is well-tolerated among patients. MTD of 10 mg/m² provided peak plasma concentrations on average exceeding 75 ng/mL. This above concentrations required in vitro and in vivo to induce significant growth inhibition in many models for various primary human tumors. Although objective responses were not observed, 15 patients had stable disease while on a q14-day schedule.

SNDX-275 displays a linear, dose-independent PK behavior within the dose-range studied (2 to 12 mg/m²). Overall, drug absorption was rapid, and in some patients the Tmax was observed as early as 30 minutes, suggesting SNDX-275 might undergo rapid gastric absorption before reaching the small intestine. The disappearance of SNDX-275 was characterized by an apparent bi-exponential decline with a T_{1/2} in plasma of approximately 50 hours—substantially longer than observed for SNDX-275 in laboratory animals. The basis for this long half-life in humans is possibly related to enterohepatic recirculation processes, suggested by the appearance of a second SNDX-275 peak around 24 to 48 hours after initial drug intake in several patients. Furthermore, the Tmax observed at 24, 48 and 60 hours suggests a substantially longer normal gastrointestinal transit time. Any hypothetical recirculation is thus likely to mask the true disposition half-life of the free drug, as has been observed previously with many other agents. SNDX-275 is only ≈80% protein bound, and there was no greater binding affinity to albumin than other plasma proteins.

The observed variability in the PK behavior of SNDX-275 with an interpatient variability in the apparent CL/F of about 40% is typical for cancer drugs administered orally. Over the dose range studied, the SNDX-275 AUC demonstrated an apparent dose-independent behavior. Body-surface area correction did not account for the interpatient variability in clearance (38.8% vs. 39.5%), suggesting that body-surface area is not a significant predictor of oral SNDX-275 pharmacokinetics and that flat-dosing regimens might be applied without compromising overall safety profiles.

### Example 3

### Pharmacokinetics in Patients with Hematologic Malignancies

A phase 1 trial of orally administered SNDX-275 was conducted in 38 adults with advanced acute leukemias. Cohorts of patients were treated with SNDX-275 initially once weekly×2, repeated every 4 weeks from 4 to 8 mg/m², and after 13 patients were treated, once weekly×4, repeated every 6 weeks from 8 to 10 mg/m². The maximum tolerated dose was 8 mg/m² at weekly×4 every 6 weeks schedule. Dose-limiting toxicities (DLTs) included infections and neurologic toxicity, manifesting as unsteady gait and somnolence. Other frequent non-DLTs were fatigue, anorexia, nausea, vomiting, hypoalbuminemia, and hypocalcemia. Treatment with SNDX-275 an increase in protein and H3/H4 acetylation, p21 expression, and caspase-3 activation in bone marrow mononuclear cells.

### Patient Eligibility and Selection

Adults, aged 18 years or older, with acute leukemia or high-risk MDS resistant to or relapsed after prior induction regimens; newly diagnosed acute leukemias in adults over age 60 with poor-risk features (antecedent hematologic disorder, poor-risk/complex karyotype); AML arising from MDS or secondary AML; acute promyelocytic leukemia (APL) patients who failed ATRA and arsenic trioxide; and chronic myelogenous leukemia (CML) in accelerated or blasé crisis or interferon-refractory CML in chronic phase were eligible for study entry provided they met the following criteria: ECOG performance status 0-2; bilirubin 1.5 times normal or less; hepatic enzymes 2 times normal or less; serum creatinine 1.5 times normal or less; and left ventricular ejection fraction 45% or higher. Complete recovery from toxicities of previous treatment, an interval of 3 weeks or more from previous chemotherapy (hydroxyurea was allowed up to 24 hours prior to SNDX-275 administration), and an interval of 1 week or more from any other growth factor therapy was required before beginning SNDX-275.

Patients were ineligible if they had peripheral blast count of 50×10⁹/L or higher; disseminated intravascular coagulation; active central nervous system (CNS) leukemia; if they were eligible for SCT; received more than 3 prior courses of induction/reinduction therapy; concomitant radiotherapy, chemotherapy, or immunotherapy; coexisting medical or psychiatric conditions that could interfere with study procedures. Pregnant or lactating women were ineligible. All patients provided written informed consent.

Complete history and medical examination were performed within 7 days of study entry. The following laboratory parameters were obtained at 3 days or less before entry: complete blood count with differential; comprehensive electrolyte panel; coagulation profile; urinalysis; bone marrow (BM) aspirate/biopsy with histochemical, cytogenic and immunophenotypic analysis; chest x-ray; electrocardiogram; surveillance cultures of throat, stool, and urine; and pregnancy test. Additional studies were performed when clinically indicated.

### Treatment Schema

Patients received SNDX-275 tablets orally with food once a week for 2 or 4 consecutive weeks depending on the dose level (DL) and followed by a two-week wash-out period. The drug was discontinued at any time for grade 3 or higher non-hematologic toxicity according to NCI CTC, Version 2, BMT criteria. The starting dose of 4 mg/m² weekly was selected based on preliminary pharmacokinetic and safety data for the same dose of SNDX-275 administered biweekly in patients with solid tumors. Using a modified Fibonacci dose escalation scheme with a dose escalation increment of 2 mg/m², the first patient cohort received SNDX-275 at 4 mg/m² for two weeks in a row of a 28-day cycle. Patients were entered on the study in cohorts of 3-6. The criteria for dose escalation were based on the safety data from the first treatment cycle. Dose was escalated to the next level if 0 of 3 patients experienced DLT. If 1 of 3 patients experienced DLT, the cohort was expanded to 6 patients. If 2 or more of 6 patients experienced DLT, no additional patients were entered at the dose. The occurrence of any DLT in 33% or more of a patient cohort defined the maximal administered dose (MAD). Once MAD was reached, an expanded cohort of patients (3-6 additional patients) was required to be treated at the highest prior well-tolerated dose level, MTD, and observed for DLT. A DLT was defined as either grade 3 or higher drug-related (possibly, probably, definitely) non-hematologic toxicity or grade 4 myelosuppression lasting 28 days or more in the absence of residual leukemia (i.e. following achievement of marrow tumor clearance). There was no limitation on the number of cycles administered as long as a patient did not have progressive disease or unacceptable toxicity. Initially, the dose escalation proceeded from 4 to 8 mg/m² of SNDX-275 given weekly for two weeks in a row of a 28-day cycle (DL 1-3). Following completion of the first three dose levels with no MTD reached, and based on early clinical observations suggesting that prolonged exposure to HDAC inhibitors or other differentiating agents may be required for their efficacy, instead of pursuing further dose escalation the schedule of drug administration was changed to once weekly for 4 weeks of a 42-day cycle and starting at 8 mg/m².

### Definition of Response

To assess response to therapy, a BM aspiration was performed weekly for 4 weeks during the first cycle, at the end of each subsequent cycle, or at any time leukemia regrowth was suspected. Complete response (CR) required a normal BM aspirate with absence of identifiable leukemia, absolute neutrophils count (ANC) of 1×10⁹/L or higher, platelet count of 100×10⁹/L or higher, and absence of blasts in peripheral blood (PB). Partial response (PR) was defined as the presence of trilineage hematopoiesis in the marrow with recovery of ANC and platelets to the above-stated levels, but with 5% to 25% blasts in the marrow. Progressive disease (PD) was defined as >50% increase in marrow or PB blasts from baseline or development of extramedullary leukemia.

### Pharmacodynamic Analysis

Serial marrow and/or peripheral blood cells were collected at weekly intervals to examine PAMC-induced changes in histone acetylation.

Changes in histone H3, H4 and protein acetylation (immunocytochemistry, multiparameter flow cytometry and western blot analysis.) Immunocytochemical analysis of histone H3 was performed on bone marrow mononuclear cells (BMMCs) as described previously. The multiparameter flow cytometric analysis of protein acetylation versus caspase-3 activation was performed on serial BM aspirates using monoclonal anti-acetylated lysine antibody (Cell Signaling Technology, Beverly, Mass.) followed by FITC-conjugated goat anti-mouse antibody (Caltag Laboratories, Burlingame, Calif.) and polyclonal PE-conjugated anti-caspase-3, active form antibody (BD Pharmingen, Sand Diego, Calif.). The analysis of protein acetylation versus p21 expression or CD 34 expression was performed using polyclonal anti-acetylated lysine antibody (Cell Signaling Technology) versus monoclonal anti-p21 antibody (BD Transduction Laboratories, San Jose, Calif.) or monoclonal anti-CD34 antibody (BD Pharmingen). Primary antibodies were detected using PE-conjugated goat anti-mouse and FITC-conjugated goat anti-rabbit antibodies (Caltag Laboratories). For staining, BMMCs were isolated using Ficoll-Paque Plus (Amersham Biosciences, Piscataway, N.J.) fixed in 0.4% paraformaldehyde, permeabilized with 0.4% Triton X-100, incubated with primary antibodies for 1 hour at room temperature, washed, incubated with secondary antibodies for 1 hour, washed and analyzed on a FACSCalibur flow cytometer (Becton Dickinson, San Jose, Calif.) using CellQuest software for acquisition and FloJo software for analysis.

For western blot analysis of histone acetylation, PBMCs and BMMCs were isolated using Ficoll-Paque Plus (Amersham Biosciences). Histones from the cells were prepared as follows: Cells were washed in 2 ml HBSS and disrupted by a 1 mL ice-cold lysis buffer A (10 mM Tris pH 7.6, 5 mM butyric acid, 1% Triton X-100, 1 mM MgCl₂, and 1 mM PMSF). Nuclei were collected by centrifugation at 14,000 rpm for 15 min. The pellet was resuspended once with 250 μL ice-cold lysis buffer B (10 mM Tris pH 7.6, 0.25 M Sucrose, 3 mM CaCl₂ and 5 mM butyric acid). Sulfuric acid was added to a concentration of 0.4 N and the tubes were incubated at 4° C. overnight. Debris was pelleted by centrifugation, and the supernatant was collected. Histones were precipitated by addition of 10 volumes of acetone and incubation at −20° C. overnight. Pellets were collected by centrifugation, briefly dried under vacuum and resuspended with dH₂O. The total protein content was determined by a bicinchoninic acid assay kit (Pierce, Rockford, Ill.). The proteins 10-30 μg were separated by 15% SDS PAGE and visualized using the antibodies for acetyl-histone H3, acetyl-histone H4, and histone H2A, all from Upstate Biotechnologies (Lake Placid, N.Y.). The immunoreactive proteins were detected using ECL western blotting analysis system (Amersham Biosciences). Radiographic films of histone acetylation were scanned and digitized (UN-SCAN-IT), Silk Scientific, UT).

### Pharmacokinetic Studies

Blood samples for pharmacokinetic studies (6 mL each) were collected in tubes containing sodium heparin at the following time points: immediately before drug administration, and at 0.5, 1, 2, 6, 12, 24, 48, 60 and 72 hours after first dose of SNDX-275. All blood samples were kept on ice and centrifuged within 2 hours of collection at 3000g for 10 minutes at 4° C. Separated plasma was immediately frozen on dry ice and then stored at −70° C. until analysis.

Concentrations of SNDX-275 in plasma were determined using a validated high-performance liquid chromatographic assay with mass-spectrometric detection. The linear range of this assay is 1 to 100 ng/mL, with a lower limit of quantitation of 0.1 ng/mL. The values for precision and accuracy, determined during each analytical run by concurrent analysis of quality control samples, were within 12% relative error.

Estimates of pharmacokinetic parameters for SNDX-275 were derived from individual concentration-time data sets by non-compartmental analysis using the software package WinNonlin version 5.0 (Pharsight Corporation, Mountain View, Calif.). The pharmacokinetic parameters of interest included peak plasma concentration (Cmax), time to peak concentration (Tmax), area under the plasma concentration versus time curve extrapolated to infinity (AUC), apparent oral clearance (CL/F), and the terminal half-life (T_{1/2, z}). Inter-individual pharmacokinetic variability was assessed with the percent coefficient of variation (CV), expressed as the ratio of the standard deviation and the observed mean. The influence of the dose level on CL/F was evaluated using the Kruskal-Wallis' one-way analysis of ranks test, followed by the Dunn's multiple comparison test for identifying statistically significant group differences. These calculations were preformed with the software package NCSS version 2001 (Number Cruncher Statistical Systems, J. L. Hintze, Kayseville, Utah).

### Results

### Patient Characteristics

A total of 39 patients with leukemia were entered into this study of SNDX-275. Patient characteristics are summarized in Table 3-1, below. One patient did not receive the drug due to elevation in liver function tests prior to drug administration. The remaining 38 patients were evaluable for toxicity and 34 for response. Median age was 65 (range 25-86), and 66.7% (26 of 39) were men. Thirty-two of 39 patients had refractory disease (82%), 18 patients (46.2%) were primary refractory, and 30 patients (76.9%) had abnormal karyotype. The median number of prior regimens was 2 (0-3). Twenty-nine patients (74.4%) received ara-C in prior treatments, 3 patients had undergone prior autologous SCT and 1 patient had undergone prior allogeneic SCT. Nine (23.1%) patients received tipifarnib (7) or phyenylbutyrate/5-azacitidine (2) as their only prior therapies.

TABLE 3-1
Patient Characteristics

| SNDX-275 | Patient | Age/ | | | |
|---|---|---|---|---|---|
| (dose level) | No. | Sex | Diagnosis | Stage of disease | Cytogenetics |
| 4 mg/m² × 2/4 | 1 | 56 F | AML, M3 | Refractory relapse 2 | 50XX, t(4; 12), t(15; 17), +4mar |
| weeks (DL1) | 2 | 69 M | AML, M2 | Primary refractory | 47XY, 20q-, +11 |
| | 3 | 70 M | AML, M1 | Primary relapse 1 | 45X, −Y |
| | 4 | 72 M | AML, M7 | Primary refractory | 40XY |
| | 5 | 49 F | AML, M0 | Primary refractory | 45XX, der(2), der(3), −7, −11, −17, |
| | | | | | der(12), +2mar |
| 6 mg/m² × 2/4 | 6 | 44 F | AML, M4 | Primary refractory | 46X, t(X; 7), inv(3) |
| weeks (DL 2) | 7 | 85 M | AML, M5 | Primary refractory | 46XY |
| | 8 | 49 M | CML-BC | Relapse 1 | 46XY, t(9; 22), t(15; 17) |
| | 9 | 65 F | AML, M5 | Relapse 1 | 46XX |
| | 10 | 54 M | AML, M1 | Refractory relapse 1 | 42XY, −5, −7, +8, +9, inv(12), −16, −17, |
| | | | | | −17, der(18), −20, −20, −21, |
| | | | | | +2mar |
| 8 mg/m² × 2/4 | 11 | 69 F | AML, M2 | Refractory relapse 1 | 46XX, 7q- |
| weeks (DL 3) | 12 | 50 F | AML, M5 2° | Relapse 1 | 45X, t(9; 11), −7, der(12)t(7; 12) |
| | 13 | 74 M | AML, M2 | Primary refractory | 45XY, −7 |
| 8 mg/m² × 4/6 | 14 | 36 F | AML, M4 | Relapse 2 | 47XX, t(4; 7), inv(9), inv(16), t(17), |
| weeks (DL 4) | | | | | +22 |
| | 15 | 72 M | AML, M2 | Primary refractory | 47XY, +8/46XY, 20q- |
| | 16 | 86 M | MDS/AML | Refractory relapse 1 | 46XY |
| | 17 | 76 F | AML, M2 | Refractory relapse 1 | 47XX, 7q-, +11 |
| | 18 | 65 F | AML, M7 | Refractory relapse 1 | 46XX |
| | 19 | 45 M | AML, M4 | Refractory relapse 2 | 46XY, inv(16)V45XY, 7q-, add(8), |
| | | | | | der(12)t(12; 17),der(17)t(12; 17)del(17) |
| | | | | | (q11), −21 |
| | 20 | 65 M | MDS/AML | Primary refractory | 46XY |
| | 21 | 66 M | AML, M0 | Primary refractory | 47XY, +13 |
| | 22 | 25 F | AML, M5 | Refractory relapse 2 | 50XX, t(3; 19), t(5), +8, +8, |
| | | | | | der(9)t(9; 11)del(9), der(11)t(9; 11), |
| | | | | | der(17)t(8: 17), +19, +mar1 |
| | 23 | 63 F | AML, M5 2° | Refractory relapse 1 | 46XX, inv(3), t(11; 22) |
| | 24 | 74 F | MDS/AML | Primary refractory | 47XX, +8 |
| | 25 | 57 M | AML, M4 | Refractory relapse 1 | 48XY, 5q-, +8, +13 |
| | 26 | 38 M | AML, M1 | Primary refractory | 45XY, del(1), add(2), add(3), |
| | | | | | inv(8), add(9), add(12), der(12), |
| | | | | | add(13), add(14), der(16)t(16; 17), −17, |
| | | | | | del(17), add(18), −19, |
| | | | | | der(20)t(9; 20), +mar |
| | 27 | 73 M | AUL | New diagnosis | 46X, −Y, t(11; 21), +13 |
| | 28 | 65 M | AML, M5 | Refractory relapse 1 | 46XY |
| | 29 | 67 M | MDS/AML | Relapse 1 | 45XY, t(1; 9), −7, 20q- |
| 10 mg/m² × 4/6 | 30 | 55 M | MDS/AML | Refractory relapse 1 | 47XY, inv(1), t(10: 15), 13q-, +21 |
| weeks (DL 5) | 31 | 73 M | AML, M6 | Primary refractory | 47XY, 7q-, der(11), −12-17, −18, |
| | | | | | add(19), t(12; 20), +3-5mar |
| | 32 | 57 M | AML, M4 | Primary refractory | 45XY, add(3), add(4), −5, 7q-, |
| | | | | | add(10), −12, add(13), del(16), −17, |
| | | | | | +2mar |
| | 33 | 70 M | MDS/AML | Primary refractory | 46XY |
| | 34 | 72 M | MDS/AML | Primary refractory | 48XY, +13, +21 |
| | 35 | 55 M | AML, M4 | Refractory relapse 1 | 46XY |
| | 36 | 67 M | AML, M5 | Relapse 1 | 48XY, +5, +7 |
| | 37 | 60 M | AML, M7 | Primary refractory | 46XY, −1, add(1), del(2), add(3), |
| | | | | | add(5), −6, del(6), −7, del(8), |
| | | | | | del(10), del(12), add(15), add(16), |
| | | | | | add(17), del(18), +1-3mar |
| | 38 | 72 M | MDS/AML | Primary refractory | 47XY, dup(4), dup(7), +8 |
| | 39 | 52 F | MDS/AML | Primary refractory | 47XY, +8 |

AML (acute myeloid leukemia),
CML-BC (chronic myeloid leukemia-blast crisis),
AUL (acute undifferentiated leukemia),
2° (secondary),
MDS/AML (AML arising from myelodysplasia)

### Toxicity

The total number of patients enrolled and cycles administered at each dose level are depicted in Table 3-2. The summary of all drug-related toxicities is provided in Table 3-3. Initially, SNDX-275 was administered weekly for two weeks followed by two weeks of rest (28-day cycle). Dose escalation progressed from DL 1 to DL 3 without the achievement of MTD. Only one patient treated at DL 2 did not complete the first cycle due to rapidly progressive disease. As depicted in Table 3-2, only DLT-grade 3 fatigue was recorded at DL 1. This patient had underlying disease-related fatigue and the contribution of SNDX-275 could not be clearly assessed.

TABLE 3-2
Dose Levels

| | No. of | | Median | |
|---|---|---|---|---|
| | patients | | No. of | |
| | (completed | Total No. of | Cycles | |
| Dose level/Schedule | 1^{st} cycle) | Cycles | (range) | DLTs |
| DL 1: 4 mg/m² × 2/4 wks | 5 (5) | 7 | 2 (1-4)* | 1# |
| DL 2: 6 mg/m² × 2/4 wks | 5 (4) | 9* | 1 | 0 |
| DL 3: 8 mg/m² × 2/4 wks | 3 (3) | 3 | 1 | 0 |
| DL 4: 8 mg/m² × 4/6 wks | 15 (9) | 27 | 1 (1-5) | 0 |
| DL 5: 4 mg/m² × 4/6 wks | 10 (5) | 12 | 1 (1-2) | 5± |

*Two patients enrolled at level 1 received each 2 subsequent cycles at level 2
#Fatigue
±Infections (3); neurologic toxicity (1) and abnormal laboratory parameters (1)

In the absence of DLTs at DL 2 and 3, the schedule of drug administration was changed to weekly for four weeks followed by two weeks rest (42-day cycle). Initially, 8 patients were treated at DL4; 2 patients did not complete the first cycle due to death (one died on day 2 from PD/leukostasis, one died on day 13 from progressive fungal pneumonia). An additional 2 patients died while on study: one on day 36 of cycle 1 from sepsis, and another with sudden death by day 20 of cycle 2, thought to be related to his underlying heart disease (history of mitral valve prolapse, aortic stenosis, atrial fibrillation and hypertension) in the setting of diarrhea and dehydration related to his chronic C. difficile colitis and pancreatic insufficiency as a consequence of previous Whipple surgery for duodenal carcinoma. Since none of the above events were considered to be SNDX-275-related, dose escalation progressed to DL 5. Five patients (50%) at DL 5 were unable to complete the first cycle of treatment due to PD (1 patient), progressive fungal pneumonia and early death on days 5 and 8 (2 patients), or overwhelming Staph aureus sepsis and ultimate death on days 16 and 36 (2 patients). An additional patient died on day 35 from PD, however, this patient developed grade 4 neurologic toxicity manifesting as a somnolence, weakness and unsteady gait, and grade 3 laboratory abnormalities such as elevated LDH, hypertriglyceridemia and hyperglycemia, possibly related to SNDX-275 and as such were considered DLTs. As shown in Table 3-4, infections such as bacteremia, sepsis and pneumonia were encountered at all dose levels and were primarily related to the underlying disease, as almost all patients were neutropenic for prolonged periods of time, underwent extensive treatments prior to SNDX-275 and had central venous catheters or previous pneumonias. However, only at DL 5, 2 overwhelming Staph aureus bacteremias were observed; and in an additional patient with bone marrow CR, grade 3 pneumonia and bacteremia in the absence of neutropenia. These episodes, although possibly explicable by underlying disease, must be also considered possibly related to SNDX-275. As such, further dose escalation was terminated and the DL 4 cohort was expanded by another 7 patients; 4 of the patients did not complete the full cycle because of PD. Two patients completed 5 cycles and 1 patient completed 2 cycles of treatment before their disease progressed with no evidence of DLT. Therefore, the MTD of SNDX-275 was 8 mg/m² administered weekly×4 every 6 weeks. Due to frequent progressive disease in the patient population, only 8 (21%) patients received 2 or more cycles of treatment.

TABLE 3-3
Adverse Events

| | All Grades | | |
|---|---|---|---|
| Adverse Events | No. of Cycles | % | Grade 3-4 (5) |
| General | | | |
| Dehydration, depression, fever, | 5 | 9 | |
| dizziness, insomnia | | | |
| Fatigue | 12 | 21 | 1 |
| Gastrointestinal | | | |
| Anorexia | 5 | 9 | |
| Nausea | 8 | 14 | |
| Vomiting | 6 | 10 | |
| Abdominal pain/gas | 1 | 2 | |
| Infections | | | |
| Neutropenic bacteremia, sepsis, | 9 | 16 | 8 (2) |
| pneumonia, other | | | |
| Laboratory | | | |
| Hypoalbuminenia | 31 | 53 | 3 |
| Hypocalcemia | 32 | 55 | |
| Hyponatremia, Hyperkalemia | 2 | 3 | |
| LDH | 1 | 2 | 1 |
| Bilirubin | 1 | 2 | |
| Glucose | 1 | 2 | 1 |
| Triglycerides | 1 | 2 | 1 |
| Hematologic | | | |
| Cytopenias | 9 | 16 | 6 |
| Neurologic | | | |
| Unsteady gait, lower extremities | 3 | 5 | 1 |
| weakness | | | |
| Somnolence/Lethargy | 1 | 2 | 1 |

TABLE 3-4
Infections

| Dose level | Bacteremia* | Sepsis | Pneumonia** |
|---|---|---|---|
| 1 | 2 | 1 | 0 |
| 2 | 2 | 0 | 1 |
| 3 | 2 | 0 | 0 |
| 4 | 3 | 1 | 5 |
| 5 | 2 | 2 | 3 |

*In most instances bacteremia was line-related and caused by Staph epidermidis. Two patients developed Staph aureus bacteremia at DL 5. There was no evidence of mucositis.
**Most frequently it was reactivation of known fungal pneumonia.

As shown in Table 3-3, frequent grade 1 and 2 toxicities were anorexia, nausea and vomiting occurring in almost one third of patients independent of dose level. Fatigue occurred in 21% of treatment cycles and more frequently at DL 4. Laboratory abnormalities such as hypoalbuminemia and accompanying hypocalcemia were recorded in more than 50% of cycles each. However, most of the patients were hypoalbuminemic at the start of the treatment and the decrease in albumin was brief, with no clinical consequences, and independent of the dose. Three patients who developed grade 3 hypoalbuminemia had grade 2 hypoalbuminemia at the start of the treatment.

### Clinical Outcome

No patients achieved CR or PR by standard criteria. The biologic effects of SNDX-275 are summarized in Table 3-5. Among the patients who experienced improvement sin ANC, the time to response and duration of response varied. While some patients experienced improvement in ANC already in the first week of treatment, some patients required 4 or more weeks to achieve response. Duration of ANC response varied from 1 to 10 weeks, and occasionally, patients with temporary improvement would experience recurrent increase in ANC in subsequent cycles.

TABLE 3-5
Biologic Effects

| Patient No. | Dose Level | Type of Response | No. of Cys |
|---|---|---|---|
| 2 | 4 mg/m² × 2/4 weeks | Bone marrow PR (>50% → 12% blasts, cycle 1) | 4 |
| | | Differentiation in myeloid lineage (ANC 504/μL → | |
| | | ANCmax 1410/μL, cycle 1) | |
| 3 | 4 mg/m² × 2/4 weeks | Stable disease (Bone marrow >20% → 11% blasts, cycle | 2 |
| | | 1) | |
| 5 | 4 mg/m² × 2/4 weeks | Stable disease | 3 |
| | (cycle 1) | | |
| | 6 mg/m² × 2/4 weeks | | |
| | (cycle 2 and 3) | | |
| 11 | 8 mg/m² × 2/4 weeks | Decreased transfusion requirements | 1 |
| 14 | 8 mg/m² × 4/6 weeks | Resolution of bone pain | 1 |
| 15 | 8 mg/m² × 4/6 weeks | Bone marrow PR (35% → 14% blasts, cycle 1) | 1.75 |
| | | Differentiation in myeloid lineage (ANC 288/μL → | |
| | | ANCmax 2052/μL, cycle 1; ANCmax 5546/μL, cycle 2) | |
| 18 | 8 mg/m² × 4/6 weeks | Differentiation in myeloid lineage (ANC 124/μL → | 1.25 |
| | | ANCmax 680/μL, cycle 1) | |
| 20 | 8 mg/m² × 4/6 weeks | Decreased transfusion requirements | 1 |
| 24 | 8 mg/m² × 4/6 weeks | Bone marrow PR (30% → 20% blasts, cycle 1) | 4.75 |
| | | Differentiation in myeloid lineage (ANC 520/μL → | |
| | | ANCmax 3637/μL, cycle 1; ANCmax 4456/μL, cycle 3) | |
| 27 | 8 mg/m² × 4/6 weeks | Stable disease (Peripheral blood 21% → 0% blasts, cycle 1 | 5 |
| | | to 3; Bone marrow 68% → 34% blasts, cycle 1, 67% blast, | |
| | | cycle 4, 87% blasts, cycle 5) | |
| 33 | 10 mg/m² × 4/6 weeks | Resolution of extramedullary chloroma | 2 |
| 34 | 10 mg/m² × 4/6 weeks | Bone marrow CR (54% → 3% blasts, cycle 1) | 2 |
| | | Differentiation in myeloid lineage (ANC 103/μL → | |
| | | ANCmax 854/μL, cycle 1; ANCmax 2929/μL, cycle 2) | |

The median baseline peripheral white blood cell (WBC) count was 3.3×10⁹/L (range 0.4-41.8×10⁹/L), with 33 of 39 (84.6%) and 10 of 39 (25.6%) patients having a baseline peripheral WBC count >1×10⁹/L and >10×10⁹/L, respectively. Fifteen (38.5%) patients had a 50% reduction in WBC counts occurring at a median 6 days (range 3-28) and 10 of 15 patients having >50% WBC counts reduction by day 7.

### Increase in Protein Acetylation

To examine whether SNDX-275 inhibited HDAC activity in leukemia cells, a comprehensive analysis of histone/protein acetylation status of PBMCs and BMMCs before and following SNDX-275 treatment was performed. An increase in histone H3 and H4 acetylation was detected by western blotting in PBMCs and BMMCs collected from 8 patients treated at DL 4 and 1 patient at DL 5. Consistent patterns of induction of histone acetylation were observed. Firs, and increase in histone H3 and H4 acetylation was evident at 8-12 hours following SNDX-275 treatment in 4 patients who had PB specimens collected at these early time points. Second, almost all patients had an increase in histone acetylation in either PBMCs or BMMCs documented by week 2-3 (day 8 and 15) of treatment. Third, in most instances, an increase in acetylation was seen for both histones H3 and H4, although there was substantial intra- and inter-patient variability. Fourth, histone acetylation increased with time and persisted for at least 2-3 weeks following last SNDX-275 administration. A similar increase in protein acetylation was also observed by flow cytometry in the majority of BM specimens obtained from patients treated at DL 4/. Of four specimens analyzed by both techniques, results were consistent for three of them. Five patients who achieved clinical response and had MCs examined for protein/histone acetylation status had an increase in acetylation. However, increase in acetylation was observed also in patients without response and the pattern of acetylation (time, intensity) appeared similar for responders and non-responders.

Furthermore, flow cytometric analysis allowed for confirmation of an increase in acetylation that occurred in CD34⁺ cells in 4 patients' specimens examined and caspase-3 activation (67% of specimens). These findings are in agreement with observed in vitro effects of SNDX-275 on leukemia cell lines and primary patient samples.

As the above assays were developed and validated later on in this study, initially samples were analyzed for histone H3 acetylation status by immunocytochemistry. An increase in histone H3 acetylation was already detected at the lowest dose level of PAMC. Additional specimens from a single patient treated at DL2 were analyzed by flow cytometry, which detected the increase in protein acetylation following SNDX-275 administration. Similar increases in histone H3 acetylation in PBMCs at all dose levels were reported in a previous SNDX-275 solid tumor study with no clear demonstration of dose-effect.

### Pharmacokinetics

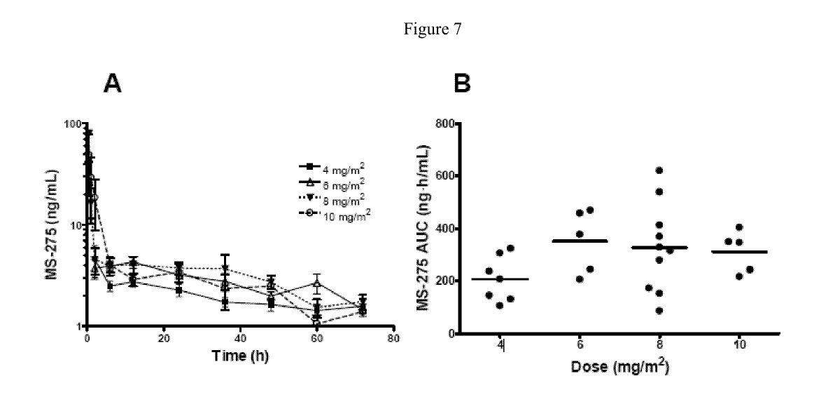

Complete pharmacokinetic data were available from 35 patients. As shown in FIG. 7A, the plasma concentration versus time profiles of SNDX-275 were very similar at each dose level and consistent with those observed previously with the drug administered at similar dose levels. For 8 patients (two at 6 mg/m², five at 8 mg/m² and 1 at 10 mg/m²), the percentage of the AUC extrapolated beyond the last sampling time point with measurable concentrations was greater than 50%. For these individuals, only Cmax and Tmax were considered. A summary of the pharmacokinetic parameters of SNDX-275 obtained as a function of DL is provided in Table 3-6.

TABLE 3-6
PK Parameters

| Dose | No. of | Cₘₐₓ | AUC | CL/F | T_{1/2} | Tₘₐₓ |
|---|---|---|---|---|---|---|
| (mg/m²) | patients | (ng/mL) | (ng.h/mL) | (L/h) | (h) | (h) |
| 4 | 7 | 4.74 ± 3.41 | 208 ± 86.1 | 40.9 ± 17.2 | 36.6 ± 18.6 | 12 |
| | | (2.20-10.7) | (106-324 | (24.7-75.8) | (16.2-64.2) | (12-24) |
| 6 | 7 | 4.87 ± 1.76 | 351 ± 121^{a} | 34.4 ± 11.5^{a} | 43.8 ± 16.4^{a} | 12 |
| | | (2.10-7.60) | (206-470) | (25.5-48.7) | (28.3-69.3) | (2-36) |
| 8 | 15 | 53.1 ± 92.4 | 328 ± 168^{b} | 66.7 ± 54.5^{b} | 33.4 ± 12.7^{b} | 1 |
| | | (2.71-302) | (86.0-620) | (25.8-209) | (16.7-53.3) | (0.5-24) |
| 10 | 6 | 53.2 ± 58.0 | 312 ± 78.8^{c} | 68.2 ± 25.4^{c} | 30.4 ± 13.9^{c} | 1 |
| | | (4.55-164) | (217-404) | (48.7-103) | (14.6-49.5) | (0.5-2) |
| All doses | 35 | NA | NA | 54.0 ± 37.2^{d} | 35.6 ± 15.0^{d} | 2 |
| | | | | (24.7-209) | (14.6-69.3) | (0.5-36) |

*Data are shown as mean and SD, with observed range in parentheses, except for Tₘₐₓ (median with range in parentheses).
Abbreviations:
Cₘₐₓ peak plasma concentration;
AUC, area under the plasma concentration versus time curve extrapolated to infinity;
CL/F, apparent oral clearance;
T_{1/2}, half-life of the terminal phase;
Tₘₐₓ, time to Cₘₐₓ
^{a}n = 5;
^{b}n = 10;
^{c}n = 5;
^{d}n = 27.

Typical for oral anticancer drugs, the interindividual pharmacokinetic variability was substantial at all dose levels, with a CV for the CL/F as high as 69.8%, suggesting varied systemic exposure of SNDX-275 during the treatment. The Tmax was highly variable, with values ranging between 30 minutes and 36 hours, median value approaching 2 hours. Since the terminal half-life was relatively consistent in all patients, exhibiting an overall mean value of 35.6±15.0 hours (CV=42.2%), this suggests that interindividual differences in the rate of gastrointestinal absorption is the main contributor to variability in the observed concentration-time profiles. For 9 patients whose Tmax of SNDX-275 was 30 minutes, the rate of drug absorption could be possibly underestimated in the absence of earlier sampling points.

The AUC reached a plateau with increasing doses of SNDX-275 (Table 3-6, FIG. 7B). Furthermore, the median CL/F of SNDX-275 was dependent on drug dose (P=0.045), with values obtained at 10 mg/m² being statistically significantly higher than those observed at the two lowest dose levels. However, because of the relatively small number of patients studied at each dose level in combination with the extensive interindividual pharmacokinetic variability, it is most likely that this dose-dependence is a spurious finding.

### Discussion

This study represents the first clinical trial of the HDAC inhibitor SNDX-275 in patients with advanced acute leukemias, mainly AML. In this cohort of patients, it was found that SNDX-275 can be safely given on a weekly schedule for 2 to 4 weeks in the dose range explored. The most frequently encountered toxicities were fatigue and gastrointestinal symptoms, such as anorexia, nausea and vomiting, occurring at all dose levels, with fatigue being more frequently documented at DL 4 and higher. These toxicities appear to be a hallmark of all HDAC inhibitors developed so far, and were noted in a study of SNDX-275 in patients with solid tumors, where they represented DLTs ad DL 12 mg/m². In contrast, however, in this study, it was not considered that these effects were dose limiting, in part because many of the patients in the cohort had baseline fatigue and nausea that correlated clinically with disease progression prior to SNDX-275 administration. Furthermore, due to advanced leukemia in this patient population and frequent early removal from the study due to disease progression, this study provides only limited information on the tolerability of SNDX-275 with chronic administration.

The MTD defined in this study was 8 mg/m²×4 weeks with a 2 week wash-out period. However, DLTs may be viewed as slightly unusual for this patient population. As already presented above, infections such as bacteremias/sepsis (line related or not) or reactivation/progression of pneumonia, which are inherent to this patient population, were encountered at all dose levels. Nonetheless, at least two infectious episodes at DL 5 occurred in patients without neutropenia or in the setting of early neutrophil recovery.

Overall, peak plasma concentrations achieved at MTD in this study and in the solid tumor study are similar and above levels required to achieve in vitro or in vivo growth inhibition of various tumors in different models.

There was substantial interindividual variability in the pharmacokinetic behavior of SNDX-275, which is not unexpected for oral anti-cancer drugs and has been reported in a previous study of SNDX-275 with patients having solid tumors. In the setting of relatively stable T_{1/2} and highly variable Tmax (30 mins to 36 hours), the individual differences in the rate of GI absorption remains the most likely explanation for observed variability. It is also conceivable that intercurrent illnesses and medications such as antibiotics administered to this patient population may in part affect the rate of GI absorption. This study also confirms that the mean T_{1/2} of 36 hours in humans is substantially longer than that observed in laboratory animals. Interestingly, the AUC in our study did not increase proportionally with the dose of SNDX-275 as the median apparent CL/F of SNDX-275 appeared to be dose-dependent and increased with higher doses of SNDX-275. This finding could be spurious due to the relatively small number of patients studied and extensive interindividual pharmacokinetic variability. This possibility is supported by previously obtained PK data from a study in which SNDX-275 was escalated from 2 to 12 mg/m² and where no clear dose dependence of PK parameters was observed.

These data demonstrate that SNDX-275 has cellular and molecular effects in AML, as evidenced by increases in histone/protein acetylation, p21 expression and caspase-3 activation in vivo, consistent with its already observed effects in vitro.

Although the MTD of SNDX-275 given weekly×4 was determined to be 8 mg/m², the observation of biological and pharmacodynamic effects at all dose levels coupled with substantial PK variability suggests that even lower doses may be effective and may be even more readily tolerated.

### Example 4

### PK of SNDX-275 in Patients with Metastatic Melanoma

An additional phase 2 trial evaluated the PK of SNDX-275 in patients with metastatic melanoma. The Cmax of 18.5 mg/mL occurred around 0.5 hours after a 7 mg dose on day 1 of cycle 1 and 23.3 ng/mL on day 1 of cycle 2. The concentration after 8 hours after the dose was approximately 2 ng/mL.

While preferred embodiments of the present invention have been shown and described herein, it will be apparent to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of treating cancer in a patient, comprising administering to the patient a first dose of 10 mg SNDX-275 during a first biweek of a biweekly dosing schedule and a second dose of 10 mg of SNDX-275 during a second biweek of the biweekly dosing cycle, wherein the biweekly dosing schedule comprises at least two consecutive biweeks.

2. The method of claim 1, wherein the first dose of SNDX-275 is administered on day 1 to day 4 of the first biweek and the second dose of SNDX-275 is administered on day 1 to day 4 of the second biweek.

3. The method of claim 2, wherein the first dose of SNDX-275 is administered on day 1 to day 3 of the first biweek and the second dose of SNDX-275 is administered on day 1 to day 3 of the second biweek.

4. The method of claim 3, wherein the first dose of SNDX-275 is administered on day 1 of the first biweek and the second dose of SNDX-275 is administered on day 1 of the second biweek.

5. The method of claim 1, further comprising administering to the patient at least one 5 mg dose of SNDX-275 after the end of the biweekly dosing cycle schedule.

6. The method of claim 5, wherein the 5 mg dose of SNDX-275 is administered to the patient on a biweekly dosing schedule, wherein a first dose of 10 mg SNDX-275 is administered to the patient during a first biweek of the biweekly dosing schedule and a second dose of 10 mg of SNDX-275 is administered to the patient during a second biweek of the biweekly dosing schedule.

7. The method of claim 6, wherein the first dose of SNDX-275 is administered on day 1 to day 4 of the first biweek and the second dose of SNDX-275 is administered on day 1 to day 4 of the second biweek.

8. The method of claim 7, wherein the first dose of SNDX-275 is administered on day 1 to day 3 of the first biweek and the second dose of SNDX-275 is administered on day 1 to day 3 of the second biweek.

9. The method of claim 8, wherein the first dose of SNDX-275 is administered on day 1 of the first biweek and the second dose of SNDX-275 is administered on day 1 of the second biweek.

10. The method of claim 1, further comprising detecting a drug-related toxicity in the patient and subsequently administering to the patient a reduced dose of SNDX-275.

11. The method of claim 10, wherein the reduced dose is 5 mg of SNDX-275 per dose.

12. The method of claim 11, wherein the reduced dose is administered to the patient on a biweekly dosing schedule, wherein a first dose of 5 mg of SNDX-275 is administered to the patient during the first biweek and a second dose of 5 mg of SNDX-275 is administered to the patient during the second biweek.

13. The method of claim 12, wherein the first dose of SNDX-275 is administered on day 1 to day 4 of the first biweek and the second dose of SNDX-275 is administered on day 1 to day 4 of the second biweek.

14. The method of claim 13, wherein the first dose of SNDX-275 is administered on day 1 to day 3 of the first biweek and the second dose of SNDX-275 is administered on day 1 to day 3 of the second biweek.

15. The method of claim 14, wherein the first dose of SNDX-275 is administered on day 1 of the first biweek and the second dose of SNDX-275 is administered on day 1 of the second biweek.

16. A method of treating cancer in a patient, comprising administering to the patient at least one dose of 10 mg of SNDX-275 and at least one subsequent dose of 5 mg of SNDX-275.

17. The method of claim 16, further comprising, after administering the 10 mg of SNDX-275 to the patient, detecting a drug-related toxicity in the patient, and subsequently administering the 5 mg dose of SNDX-275 to the patient.

18. The method of claim 17, wherein the 10 mg dose of SNDX-275 is administered as part of a biweekly dosing schedule, wherein a first dose of 10 mg is administered during a first biweek and optionally a second dose of 10 mg is administered during a second biweek.

19. The method of claim 16, wherein the 10 mg dose of SNDX-275 is administered as part of a biweekly dosing schedule, wherein a first dose of 10 mg of SNDX-275 is administered during the first biweek, a drug-related toxicity is then detected, and a second dose of 5 mg of SNDX-275 is administered during the second biweek.

20. The method of one of claims 1-19, wherein the mean area under the plasma concentration curve of SNDX-275 is about 100 ng·h/mL to about 400 ng·h/mL.

21. The method of one of claims 1-20, wherein the mean maximum plasma concentration of SNDX-275 is about 1 to about 60 ng/mL.

22. A method of treating cancer in a patient, comprising administering to the patient a first dose of 5 mg SNDX-275 during a first biweek of a biweekly dosing schedule and a second dose of 5 mg of SNDX-275 during a second biweek of the biweekly dosing cycle, wherein the biweekly dosing schedule comprises at least two consecutive biweeks.

23. The method of claim 22, wherein the first dose of SNDX-275 is administered on day 1 to day 4 of the first biweek and the second dose of SNDX-275 is administered on day 1 to day 4 of the second biweek.

24. The method of claim 23, wherein the first dose of SNDX-275 is administered on day 1 to day 3 of the first biweek and the second dose of SNDX-275 is administered on day 1 to day 3 of the second biweek.

25. The method of claim 24, wherein the first dose of SNDX-275 is administered on day 1 of the first biweek and the second dose of SNDX-275 is administered on day 1 of the second biweek.

26. The method of one of claims 22-24, wherein the mean area under the plasma concentration curve of SNDX-275 is about 150 ng·h/mL to about 350 ng·h/mL.

27. The method of one of claims 22-25, wherein the mean maximum plasma concentration of SNDX-275 is about 1 to about 50 ng/mL.

28. A method of treating cancer in a patient, comprising administering to the patient a first dose of 7 mg SNDX-275 during a first biweek of a biweekly dosing schedule and a second dose of 7 mg of SNDX-275 during a second biweek of the biweekly dosing cycle, wherein the biweekly dosing schedule comprises at least two consecutive biweeks.

29. The method of claim 28, wherein the first dose of SNDX-275 is administered on day 1 to day 4 of the first biweek and the second dose of SNDX-275 is administered on day 1 to day 4 of the second biweek.

30. The method of claim 29, wherein the first dose of SNDX-275 is administered on day 1 to day 3 of the first biweek and the second dose of SNDX-275 is administered on day 1 to day 3 of the second biweek.

31. The method of claim 30, wherein the first dose of SNDX-275 is administered on day 1 of the first biweek and the second dose of SNDX-275 is administered on day 1 of the second biweek.

32. The method of one of claims 28-31, wherein the mean area under the plasma concentration curve of SNDX-275 is about 100 ng·h/mL to about 400 ng·h/mL.

33. The method of one of claims 28-32, wherein the mean maximum plasma concentration of SNDX-275 is about 1 to about 60 ng/mL.

34. A method of treating cancer in a patient, comprising administering to the patient a first dose of 3 mg SNDX-275 during a first biweek of a biweekly dosing schedule and a second dose of 3 mg of SNDX-275 during a second biweek of the biweekly dosing cycle, wherein the biweekly dosing schedule comprises at least two consecutive biweeks.

35. The method of claim 34, wherein the first dose of SNDX-275 is administered on day 1 to day 4 of the first biweek and the second dose of SNDX-275 is administered on day 1 to day 4 of the second biweek.

36. The method of claim 35, wherein the first dose of SNDX-275 is administered on day 1 to day 3 of the first biweek and the second dose of SNDX-275 is administered on day 1 to day 3 of the second biweek.

37. The method of claim 36, wherein the first dose of SNDX-275 is administered on day 1 of the first biweek and the second dose of SNDX-275 is administered on day 1 of the second biweek.

38. The method of one of claims 1-19, wherein the mean area under the plasma concentration curve of SNDX-275 is about 100 ng·h/mL to about 350 ng·h/mL.

39. The method of one of claims 1-20, wherein the mean maximum plasma concentration of SNDX-275 is about 1 to about 50 ng/mL.

40. A method of treating cancer in patient, comprising administering a first dose of from 2 to 6 mg/m² of SNDX-275 on a first day of an at least 28-day dosing cycle, a second dose of from 2 to 6 mg/m² of SNDX-275 on a second day of the at least 28-day dosing cycle and a third dose of from 2 to 6 mg/m² on a third day of the at least 28-day dosing cycle.

41. The method of claim 40, wherein the first dose of SNDX-275 is 2 mg/m².

42. The method of claim 41, wherein the second dose of SNDX-275 and the third dose of SNDX-275 are each 2 mg/m².

43. The method of claim 40, wherein the first dose of SNDX-275 is 4 mg/m².

44. The method of claim 43, wherein the second dose of SNDX-275 and the third dose of SNDX-275 are each 4 mg/m².

45. The method of claim 40, wherein the first dose of SNDX-275 is 6 mg/m².

46. The method of claim 45, wherein the second dose of SNDX-275 and the third dose of SNDX-275 are each 6 mg/m².

47. The method of claim 40, wherein the first dose of SNDX-275 is administered on day 1 to day 7 of the at least 28-day dosing cycle and the second dose of SNDX-275 and the third dose of SNDX-275 are each administered on day 8 to day 28 of the at least 28-day dosing cycle.

48. The method of claim 47, wherein the first dose of SNDX-275 is administered on day 1 to day 7 of the at least 28-day dosing cycle and the second dose of SNDX-275 and the third dose of SNDX-275 are each administered on day 8 to day 21 of the at least 28-day dosing cycle.

49. The method of claim 48, wherein the first dose of SNDX-275 is administered on day 1 to day 4 of the at least 28-day dosing cycle, the second dose of SNDX-275 is administered on day 8 to day 11 of the at least 28-day dosing cycle and the third dose of SNDX-275 is administered on day 15 to day 18 of the at least 28-day dosing cycle.

50. The method of claim 49, wherein the first dose of SNDX-275 is administered on day 1 to day 3 of the at least 28-day dosing cycle, the second dose of SNDX-275 is administered on day 8 to day 10 of the at least 28-day dosing cycle and the third dose of SNDX-275 is administered on day 15 to day 17 of the at least 28-day dosing cycle.

51. The method of claim 50, wherein the first dose of SNDX-275 is administered on day 1 of the at least 28-day dosing cycle, the second dose of SNDX-275 is administered on day 8 of the at least 28-day dosing cycle and the third dose of SNDX-275 is administered on day 15 of the at least 28-day dosing cycle.

52. The method of one of claims 40-51, wherein the mean area under the plasma concentration curve of SNDX-275 is about 100 ng·h/mL to about 350 ng·h/mL.

53. The method of one of claims 40-52, wherein the mean maximum plasma concentration of SNDX-275 is about 1 to about 50 ng/mL.

54. A method of treating cancer in a patient, comprising administering to the patient two doses of about 2 to about 10 mg/m² each of SNDX-275 over the course of a 4 week treatment cycle, wherein a first dose of SNDX-275 is administered during week 1, a second dose of SNDX-275 is administered during week 2, and no dose of SNDX-275 is administered during each of weeks 3 and 4.

55. The method of claim 54, wherein the first dose is about 2 mg/m².

56. The method of claim 55, wherein the second dose is about 2 mg/m².

57. The method of claim 54, wherein the first dose is about 4 mg/m².

58. The method of claim 57, wherein the second dose is about 4 mg/m².

59. The method of claim 54, wherein the first dose is about 6 mg/m².

60. The method of claim 59, wherein the second dose is about 6 mg/m².

61. The method of claim 54, wherein the second dose is about 8 mg/m².

62. The method of claim 61, wherein the second dose is about 8 mg/m².

63. The method of one of claims 54-62, wherein the mean area under the plasma concentration curve of SNDX-275 is about 150 ng·h/mL to about 350 ng·h/mL.

64. The method of one of claims 54-63, wherein the mean maximum plasma concentration of SNDX-275 is about 1 to about 50 ng/mL.

65. The method of one of claims 54-65, wherein the mean time to maximum plasma concentration of SNDX-275 is about 1.5 to about 6 hours.

66. A method of treating cancer in a patient, comprising administering to the patient four doses of about 2 to about 10 mg/m² each of SNDX-275 over the course of a 6 week treatment cycle, wherein a first dose of SNDX-275 is administered during week 1, a second dose of SNDX-275 is administered during week 2, a third dose of SNDX-275 is administered during week 3, a fourth dose is administered during week 4, and no dose of SNDX-275 is administered during each of weeks 5 and 6.

67. The method of claim 66, wherein the first dose is about 2 mg/m².

68. The method of claim 67, wherein each of the second, third and fourth doses is about 2 mg/m².

69. The method of claim 66, wherein the first dose is about 4 mg/m².

70. The method of claim 69, wherein each of the second, third and fourth doses is about 4 mg/m².

71. The method of claim 66, wherein the first dose is about 6 mg/m².

72. The method of claim 71, wherein each of the second, third and fourth doses is about 6 mg/m².

73. The method of claim 66, wherein the first dose is about 8 mg/m².

74. The method of claim 73, wherein each of the second, third and fourth doses is about 8 mg/m².

75. The method of claim 66, wherein the second dose is about 10 mg/m².

76. The method of claim 75, wherein each of the second, third and fourth doses is about 10 mg/m².

77. The method of one of claims 66-76, wherein the mean area under the plasma concentration curve of SNDX-275 is about 300 ng·h/mL to about 350 ng·h/mL.

78. The method of one of claims 66-77, wherein the mean maximum plasma concentration of SNDX-275 is about 40 to about 60 ng/mL.

79. The method of one of claims 66-78, wherein the mean time to maximum plasma concentration of SNDX-275 is about 0.5 to about 6 hours.

80. The method of one of claims 1-79, wherein the SNDX-275 is administered orally.

81. The method of one of claims 1-79, wherein the SNDX-275 is administered orally in the form of one or more tablets.

82. The method of one of claims 1-79, wherein the SNDX-275 is administered orally in the form of 0.5, 1, 2, 3, 4, 5, 6, 7, 8, 9 or 10 mg tablets or a suitable combination of 2 or more thereof.

83. A method of treating cancer in a patient, comprising administering to the patient a fixed dose of about 1 to about 10 mg of SNDX-275 one time per week.

84. The method of claim 83, wherein the fixed dose is about 1 mg, 2 mg, 3 mg, 4 mg, 5 mg, 6 mg, 7 mg, 8 mg, 9 mg or 10 mg of SNDX-275, administered one time per week.

85. The method of claim 83, wherein the fixed dose is about 1 to about 6 mg of SNDX-275, administered one time per week.

86. The method of claim 83, wherein the fixed dose is about 1 mg, 2 mg, 3 mg, 4 mg, 5 mg or 6 mg of SNDX-275, administered one time per week.

87. A method of treating cancer in a patient, comprising administering to the patient a fixed dose of about 1 to about 10 mg of SNDX-275 one time every other week.

88. The method of claim 83, wherein the fixed dose is about 1 mg, 2 mg, 3 mg, 4 mg, 5 mg, 6 mg, 7 mg, 8 mg, 9 mg or 10 mg of SNDX-275, administered one time every other week.

89. The method of claim 83, wherein the fixed dose is about 1 to about 6 mg of SNDX-275, administered one time every other week.

90. The method of claim 83, wherein the fixed dose is about 1 mg, 2 mg, 3 mg, 4 mg, 5 mg or 6 mg of SNDX-275, administered one time every other week.

91. The method of one of claims 83-90, wherein the mean area under the plasma concentration curve of SNDX-275 is about 1 ng·h/mL to about 400 ng·h/mL.

92. The method of one of claims 83-91, wherein the mean maximum plasma concentration of SNDX-275 is about 40 to about 60 ng/mL.

93. The method of one of claims 83-92, wherein the mean time to maximum plasma concentration of SNDX-275 is about 0.5 to about 24 hours.

94. The method of one of claims 83-93, wherein the SNDX-275 is administered orally.

95. The method of one of claims 83-94, wherein the SNDX-275 is administered orally in the form of one or more tablets.

96. The method of one of claims 83-95, wherein the SNDX-275 is administered orally in the form of 0.5, 1, 2, 3, 4, 5, 6, 7, 8, 9 or 10 mg tablets or a suitable combination of 2 or more thereof.
